SEQUENCE LISTING (38 sequences)

SEQ ID NO: 1 - DNA, 4206 bp, Homo sapiens
agtgcccgccactcccactgagattgagagacgcggcaaggaggcagcctgtggaggaactgggtaggatttaggaacgcaccgtgcacatgcttggtggtcttgttaagtggaaactgctgctttagagtttgtttggaaggtccgggtgactcatcccaacatttacatccttaattgttaaagcgctgcctccgagcgcacgcatcctgagatcctgagcctttggttaagaccgagctctattaagctgaaaagataaaaactctccagatgtcttccagtaatgtcgaagtttttatcccagtgtcacaaggaaacaccaatggcttccccgcgacagcttccaatgacctgaaggcatttactgaaggagctgtgttaagttttcataacatctgctatcgagtaaaactgaagagtggctttctaccttgtcgaaaaccagttgagaaagaaatattatcgaatatcaatgggatcatgaaacctggtctcaacgccatcctgggacccacaggtggaggcaaatcttcgttattagatgtcttagctgcaaggaaagatccaagtggattatctggagatgttctgataaatggagcaccgcgacctgccaatttcaaatgtaattcaggttacgtggtacaagatgatgttgtgatgggcactctgacggtgagagaaaacttacagttctcagcagctcttcggcttgcaacaactatgacgaatcatgaaaaaaacgaacggattaacagggtcattcaagagttaggtctggataaagtggcagactccaaggttggaactcagtttatccgtggtgtgtctggaggagaaagaaaaaggactagtataggaatggagcttatcactgatccttccatcttgttcttggatgagcctacaactggcttagactcaagcacagcaaatgctgtccttttgctcctgaaaaggatgtctaagcagggacgaacaatcatcttctccattcatcagcctcgatattccatcttcaagttgtttgatagcctcaccttattggcctcaggaagacttatgttccacgggcctgctcaggaggccttgggatactttgaatcagctggttatcactgtgaggcctataataaccctgcagacttcttcttggacatcattaatggagattccactgctgtggcattaaacagagaagaagactttaaagccacagagatcatagagccttccaagcaggataagccactcatagaaaaattagcggagatttatgtcaactcctccttctacaaagagacaaaagctgaattacatcaactttccgggggtgagaagaagaagaagatcacagtcttcaaggagatcagctacaccacctccttctgtcatcaactcagatgggtttccaagcgttcattcaaaaacttgctgggtaatccccaggcctctatagctcagatcattgtcacagtcgtactgggactggttataggtgccatttactttgggctaaaaaatgattctactggaatccagaacagagctggggttctcttcttcctgacgaccaaccagtgtttcagcagtgtttcagccgtggaactctttgtggtagagaagaagctcttcatacatgaatacatcagcggatactacagagtgtcatcttatttccttggaaaactgttatctgatttattacccatgaggatgttaccaagtattatatttacctgtatagtgtacttcatgttaggattgaagccaaaggcagatgccttcttcgttatgatgtttacccttatgatggtggcttattcagccagttccatggcactggccatagcagcaggtcagagtgtggtttctgtagcaacacttctcatgaccatctgttttgtgtttatgatgattttttcaggtctgttggtcaatctcacaaccattgcatcttggctgtcatggcttcagtacttcagcattccacgatatggatttacggctttgcagcataatgaatttttgggacaaaacttctgcccaggactcaatgcaacaggaaacaatccttgtaactatgcaacatgtactggcgaagaatatttggtaaagcagggcatcgatctctcaccctggggcttgtggaagaatcacgtggccttggcttgtatgattgttattttcctcacaattgcctacctgaaattgttatttcttaaaaaatattcttaaatttccccttaattcagtatgatttatcctcacataaaaaagaagcactttgattgaagtattcaatcaagtttttttgttgttttctgttcccttgccatcacactgttgcacagcagcaattgttttaaagagatacatttttagaaatcacaacaaactgaattaaacatgaaagaacccaagacatcatgtatcgcatattagttaatctcctcagacagtaaccatggggaagaaatctggtctaatttattaatctaaaaaaggagaattgaattctggaaactcctgacaagttattactgtctctggcatttgtttcctcatctttaaaatgaataggtaggttagtagcccttcagtcttaatactttatgatgctatggtttgccattatttaataaatgacaaatgtattaatgctatactggaaatgtaaaattgaaaatatgttggaaaaaagattctgtcttatagggtaaaaaaagccaccgtgatagaaaaaaaatctttttgataagcacattaaagttaatagaacttactgatattcctgtctagtggtataatatctcaggaatcttggctgagggtttggaactgtgggtagagtagagggccaggagtccagtaatagaattcttgcaccatttctggaacattctagctctgggaggtcacgtaaccttcttggggtagttcagtggtttagtggtttataatccaggtgtgcgtcagaatcatctgaggaactttgctaaaatacaaaaatctggcctaagtagctccagatctaccttcataaaggaatctgaccactcctggatttggtaatttccaagttctgaaaattttacttaggatttaataactattaacatctgtccctacataggttttctttcctacttatataccttatgttctcttcattctaaccttcatcagtaatagggaaatgttttaattttatttttttagttgaagggtaatgtaccaaaaaatatagttcagtgaattaaaatgaacacacatgtgcaaccatcaattcaggtcaagaaatagaagattgtagcacacaaaagcctactcagccattctcccagtcactacttccttccttacccctgggttatttttgaaatgacacttgatgtatttccctctgttgctgttatgagaacattgctacagccaagtgttgtgtttctgtgtgcataggttgatacttaattatctccccactttttaataaacttttaatttggaaataattttagattgacagaaaagttgcaaagatagtgaggaaagttcctgtctactctttgctcagcttcccttaatgttaacattttatatagcaagatgcatttgtcaaagctaacaagttaacattggtacaatcactgttaattaaactgcacacaatattcagatttcaccacttttccactaatattctttcattgttctaggattcaattcaggagaccacatttcatctagccctcttttttaaaagtaaatacttttcagcacttacaggagttaactgagctggggcatcatggtgtatagacgccctgacactggtcatcttggaattcatttagtttgtcagtgggtgccctgacattctgtcacaacatcaatttgggaacatggcattatatttttatctttgaacttttttctttttggatgacatttgattaatgcgtcatcttggaacacattatcttttttcttggttatgtgatcaggaagattaatcagtttttcctgttcttggtataattcctgcttttcacatacctgtcccttacagttctctatatatacccttcccttattacacagagagaaatatctatctatactttttacacaaaatatacttcaaaagaaacaaaacagccacaattattaactttttaaataaatgagaatttaattata

SEQ ID NO: 2 - DNA, 3632 bp, Homo sapiens
actctgggagcacagagagctcaggtagcctgcctagatggcggcgcgcaccctgggccgcggcgtcgggaggctgctgggcagcctgcgagggctctcggggcagcccgcgcggccgccgtgcggggtgagcgcgccgcgcagggcggcctcgggaccctcgggcagcgctcccgcagttgcagcagcagcagcacagccaggctcgtatcccgcgctgagtgcacaggcagcccgggagccggccgccttctgggggcctctggcgcgggacactctcgtgtgggacaccccctaccacaccgtctgggactgcgacttcagcactggcaagatcggctggttcctgggaggccagttaaatgtctctgtcaactgcttggaccagcatgttcggaagtcccccgagagcgttgctttgatctgggagcgcgatgagcctggaacggaagtgaggatcacctacagggaactactggagaccacgtgccgcctggccaacacgctgaagaggcatggagtccaccgtggggaccgtgttgccatctacatgcccgtgtccccattggctgtggcagcaatgctggcctgtgccaggatcggagctgtccacacagtcatctttgctggcttcagtgcagagtccttggctgggaggatcaatgatgccaagtgcaaggtggttatcaccttcaaccaaggactccggggtgggcgcgtggtggagctgaagaaaatagtggatgaggctgtgaagcactgccccaccgtgcagcatgtcctggtggctcacaggacagacaacaaggtccacatgggggatctggacgtcccgctggagcaggaaatggccaaggaggaccctgtttgcgccccagagagcatgggcagtgaggacatgctcttcatgctgtacacctcagggagcaccggaatgcccaagggcatcgtccatacccaggcaggctacctgctctatgccgccctgactcacaagcttgtgtttgaccaccagccaggtgacatctttggctgtgtggccgacatcggttggattacaggacacagctacgtggtgtatgggcctctctgcaatggtgccaccagcgtcctttttgagagcaccccagtttatcccaatgctggtcggtactgggagacagtagagaggttgaagatcaatcagttctatggcgccccaacggctgtccggctgttgctgaaatacggtgatgcctgggtgaagaagtatgatcgctcctccctgcggaccctggggtcagtgggagagcccatcaactgtgaggcctgggagtggcttcacagggtggtgggggacagcaggtgcacgctggtggacacctggtggcagacagaaacaggtggcatctgcatcgcaccacggccctcggaagaaggggcggaaatcctccctgccatggcgatgaggcccttctttggcatcgtccccgtcctcatggatgagaagggcagcgtcgtggagggcagcaacgtctccggggccctgtgcatctcccaggcctggccgggcatggccaggaccatctatggcgaccaccagcgatttgtggacgcctacttcaaggcctacccaggctattacttcactggagacggggcttaccgaactgagggcggctattaccagatcacagggcggatggatgatgtcatcaacatcagtggccaccggctggggaccgcagagattgaggacgccatcgccgaccaccctgcagtaccagaaagtgctgtcattggctacccccacgacatcaaaggagaagctgcctttgccttcattgtggtgaaagatagtgcgggtgactcagatgtggtggtgcaggagctcaagtccatggtggccaccaagatcgccaaatatgctgtgcctgatgagatcctggtggtgaaacgtcttccaaaaaccaggtctgggaaggtcatgcggcggctcctgaggaagatcatcactagtgaggcccaggagctgggagacactaccaccttggaggaccccagcatcatcgcagagatcctgagtgtctaccagaagtgcaaggacaagcaggctgctgctaagtgagctggcaccttgtggggctcttgggatgggcgggcacccaagccctggcttgtccttcccagaaggtacccctgaggttggcgtcttcctacgtcccagaagcagcccccaccccacacatgacccacaccgccctcacgtgaagctgggctgagagccctttctcccatccattggaggtcccaggagtgtcacccatggagaggctatgcgacatggctagggctggttctgccatctgagtttggtttcctggaatgaaaaggcattgccatctccattcctctgccctcttgagccagcacaggaaggtgaggccctgggatagcgcgcctgctcagataacacagagctagttagctagtagcaaccgtgttttctccagatctgtctagatacaaaggtcagaaatcttatttttatacttttatattgtggaagaacagcatgcaacactcacatgtagtgtgtggatttacttgaacatgttctttttaacatgtagttatgaaaatctccttttttgcctctactggtgaggaaacatgaggatcagaggccacatttttaattattgttagtgtatttggaagtctgaattggagatgtttgtacctctgtctaaacagttcccttgagaacttccaagcctccggcatcttttcctggtgagtgtttctcctgtgcttggttgtgtataatggagctaactcctaagcggtggggtgaatgtggccgccttagttctgaagctactccagttatgttctgtttcttcaagctgtgatccagaaagatttttgtgcccccagatgcctcttgataggagaggcaacatactccaaatagttgggttcttcagggaagctattagaaactcaggtgacttgttagagcactaacttggtcagagccaaatcctggcaaacgctgcctgaccttcactctgtggttggggcggtgagaaccactgaggtccaatgatgagacttggaggtctggatccagtctctctttgttttaatgtgacttaggtgctgtcaacattagcaagataatggaaatcacgacgccagtgggtgcttacctccctgctaggcatgcaggggctggcggttggcaggggaaggaggcccagtgagccgggtcccttaggggagggagagtttgtcctctttgccccacagtctacccttcagggccttgtggcagtgccagtgttcggggggtgtctgggccactgagtacccactcggtcgtggttgtgctggcctcttgggtgagtgaacctgtgaagcccaggaggtggtgttggctgcagggtacacaaatactgagtggtggtcttttgttacaggcttagcaacaaagctgtgccctgggcatggggggctgtagtgtagctacagttgtgcgtttgtgaaatggcttagctttccatgttgctgagaggaacctggacatggtcccgggcatctgaatgatctgtaggggagggagttcaaataaagctttattttgttca

SEQ ID NO: 3 - DNA, 6845 bp, Homo sapiens
agtccgcctgctactcggtcccggcgctgggctgaggggaggggttgtcttaaaagtctctccttccccctgtaggggcggccggcgagtcccagtgagagcggagggtgccagaggtagggggccgagaaacaaagttcccggggcttcctccggggccgcggtcggggctgcgcgtttgaccgcccccctcctcgcgaaggcaatggcttccaaactcctgcgcgcggtcatcctcgggccgcccggctcgggcaagggcaccgtgtgccagaggatcgcccagaactttggtctccagcatctctccagcggccacttcttgcgggagaacatcaaggccagcaccgaagttggtgagatggcaaagcagtatatagagaaaagtcttttggttccagaccatgtgatcacacgcctaatgatgtccgagttggagaacaggcgtggccagcactggctccttgatggttttcctaggacattaggacaagccgaagccctggacaaaatctgtgaagtggatctagtgatcagtttgaatattccatttgaaacacttaaagatcgtctcagccgccgttggattcaccctcctagcggaagggtatataacctggacttcaatccacctcatgtacatggtattgatgacgtcactggtgaaccgttagtccagcaggaggatgataaacccgaagcagttgctgccaggctaagacagtacaaagacgtggcaaagccagtcattgaattatacaagagccgaggagtgctccaccaattttccggaacggagacgaacaaaatctggccctacgtttacacacttttctcaaacaagatcacacctattcagtccaaagaagcatattgaccctgcccaatggaagaaccaggaagatgtggtcattcattcaatagtgtgtgtagtattggtgctgtgtccaaattagaagctagctgaggtagcttgcagcatcttttctagttgaaatggtgaactgataggaaaacaaatgagtagaaagagttcatgaagaggccctcctctgcctttcaaaaggctggtcacctacacatgtttaaggtgtctctgcacatgtctcaagcccatcacaagaaagcaagtacagtgtggatttcaaatggtgtgtaacttcagctccagctggtttttgacagctgttgctgtggtaatatttttgacatgtgatggtgatagtctctggttctccccatccccacaaaggctgttgaaccacagcaccaggaagcctgagaatgaatcctgagggctctagcccaggctttgtcccaggctttctggtgtgtgccctcctggtaacagtgaaattgaagctacttactcatagtggttgtttctctggtcttgagtgactgtgtccacagttcatttttttccggtaggaataactccttttctacatccacgctccatagagtctctccttttcagacatcctgggatgaaagaatttggcttttttttttctttttttttttggacatctgttttcactcttaggcttttaaacaatagttattgcttttatccctctcagattctaataactgagagcgatggggctatattgaatctctgtatgcactgagaactgagctatgaagaggatcttattaaactgctggtctgactttatggattgacactgttcctttcttttattgtgaaaaaaaaaaaaaaccctgaaagtcttgggaaccccctaaagtcttttgggaatcctcaaaaagcatgggaagttaagtatttagctacataaatgttgtaagatcatatcttatgtatagaagtaataagaccatttggaattactggactaattgaatagttaaggtttctattcgggacaataaaatgtattttgaaagtgctgctaactattgatgctgacagtgtttcactcctatgagtgacccaaacatattataaatatgtggtaaagggaatggagcctgtggggttgagcagaatgttgtactagctgtgcctggactgagtataacagctttatgattatgagaaaacaaattctttattttttttttctgttccaaagattcatcctatggggtggccataaagtctagaattagatactaatattttgtcattcattataacatatcaataaaccatttgttaaaagatttgcctggtttccagacttggtggccaccttgaataattcttgctgtcttctgggaaggatgatgaaatttattcctgctgccttaaaaatatgtatcccttcttcacccatcatgactgtccccagtgagtgtcctttactattcttgggagtgactcctgtctaacttttcatactggcgagaagaaaagaagcctattttaacactttagtggtgttgaaacacattacttactttctgaagatgtcccagtgaatcctctgtcaattcactgccatatgtaatctatatgataaggaatgcatcttccttctaagtactgcccaaactcttgccagctcctctcccattgtcccttcatgtgaatatttcttggctaccttagtggaaatatagatcagttttctccccatccatcctctcaaacataatgagattgtttactttttagatttatgcagtgaaaatgcccagtcaggtctgaatcgtcagtgcattatattgactctgagcactttagaatttagagttgcaattgaatgccagctgtggagatggggtgcatatcagatatataaataaagctcaggtttgctagggaaccaggtatagagaaaaataagtctgatatgaggaaaattgcacaatttagagtagttatgccgtagagaaaatttccacaaactaggaaatgtagagagttattctatagaatactcaaaagaggaaagtatgtgatttttggaaacaggaaaatcttcaaacttctttcttcacttccctttgtgtttagctgaccctccaatgtgatcattgcctttggagtttgggagaggtacgggaagtggcctgatccctgcttccatacttcactcctccatccatccttccctccctcttcccctccagctaaatggacaattctagccaacattgagtcactcaataagtctcaacagtgggtgtgtttgctgagattgtccagcggttgagcagtttggtctcacctccctcgctagttgagaccaaaaagagacaaataactttttcatggtctttgaaacataatgcttatttcgtggtcaatggctttaaaaaaatctgtttcttgttttcttcaacaaactcactagttttcccttaaatgatattgtaaaaattaaagtaatcttgaaaatgttttgacaaaagtaaaattaaagggacatcttttcttgttttgttttttttttttctattgccacacatgaccgttccttcacctttaagcaaagagagtggttcagatggtttctaagatgccaacctgacctcgcattctgtcattctacccagctcttaattcaatttgcttccattatcctaacaggcttctttcttacttagaacttggaaaggctgctgtatttaataccctccaacactaacgcagacttaagataggtactgtttattgaaaacctactgagtgaaatgtgcggttttaggaccttcataaacatctcatttaatctttctagcatcctgtgaaacagccatgatttcacgttgataaacaaagaagacaggggtcccagggatgtgaagcatcttgcccaggcttctgctgctggtgaccagtgtagccaggactccagcccaggttttcctgactcagaagactgagctttttcctggatgttattaatagctaattgtgtccaagcaaccaagggccttgagtctgcttggttctgcttatggcctcacatcaagaaatggagctagtccatgtctgtagtcccaatgctttgggaagccatgatgggaaggttgtcggaggccaaaagttcaagaccaggctgggcaatatcacaagactccatctctacggaaaagtaaaaaattagccagtcatggtggtgtacacttatggtcctagttactcaggagacttaggcaggaggattgcttgatcctaggaattcgaggctgcagtgagctatgattgcacctctgcacccaagcctgggcgacacagcgagaccctctctcttaaaaaaaaaaaatagcagagctcaccaaagtgatgttcacctttttatgacattcctttttcttagcttaagaaaagaaagctgctagatgagagtcttagttttcctgcataagacctcctttatgaatagaataaaagactgtcaaagtaggctgggcttgggcccaggctaatctatgaaggaagcaagctcgtgttccttacctatccttttggtgtccattggattgtgccccgaagtggcctttacccttgagccgtccccagccatggtgctcacacataggcttttgagctccttggagctatccagatcctgctcacttttccttcctgagatcagaacaaatcacccccttactcccactccaaacaaggccttgatgataaactaatccttcctaaaatgctggtaggtaaacaagcaatgatgaagcattgaacacaggttaactcctgacttttgtaccattgtctattccattacacattaacatgactctgaatgccagatccaaacctttgcccaccatctgcttgtcgtgcaacagttgaggcagtaaccaggggagattcacttcctgtcttgtccttccccagggatcacccccctgctgccctctagcagccaaactcagatgagttccattgttaccctaggtgtgcccatctctttggtagggaaggagaaaggtaagaatagccatcagtgaggaaggattcttggagcgaggagccactgtggtttttcctgctatttaagatgttgagaccggataactttagaaagatacctgcacaaacccataaatagtgcttttataaagtttagttcaccggaacctgagttcagtatttgacattagctttttgtccaaagagttgaagcctgctggaggtctttgctcaaataataaataccacatatttccaagtgtgttcaggtataggcactaggtactgtctgtttacttcatgttaggcacattacatgcattggctaatcaaatcctcatcaattacatatgtaataatctaaacttgcctccttgtattataaatggaaataatcctgtttatttaaacgggttttcatgtacctgtagggattaggaaactcaaatggcctttttaatacctttccctagtttgagctccctgttctctttaacagataaaacaacatatttgcttcagcctggaatctgtttttggtgctttggtgcagagacaggaaatgggcactcagagtcacactggtagttgcacactgtatctacagagggcgtgtctcatctgtactctgctgggttacaggatttcagtaggtatttgtgtccacctgagaattctgtttattacctttcatttgacagtgtctttcctttctgcagttgattttgctagagaggcaattcataaggtgaggtcctgttcatagtatgacttgctttctcaatatctccttcaatttttagtaactcttggtctatttggtgtctttaaaaaaaataacctagtaataaagacttcttttaatgtggaaatgtggtctggtagtaagttatttctttccacatgtaactgacccaatctggtttccaaatgagaagtgtgcaggccccagaggttgagaagccatatttcaactgtgaaaaaaatctgcttcctgcatctgttgaaatatagttgttcatacttgccatcccttatctttcttgtaacaatttgcacagttcttgccagaataaatgccattatctgtatgtttcagggagttccccaatttgatcatttttgtgtgtgtgtggtgtgtgtgtgagagagagagatactgcagtaaaacatttctaaaggatgaaagctcttgtatggcatagatatgaattccttcctctggtaataattaggttattcccagaagcacagtgtcattctttaaataaaagctttcctgtttaaagcttttcaaaggagcagaccaccttgaagattccccctagggttgatatgtgtctaattcattttataaaaattattcttgtcttcattttaaagctttggctatatagtcagaaatgtcctaaataacaaactattttgtatttaatttagggaagactaaagggaagaaaaatgaaaactcagtctttatgtaagctccaaggatattagggcttaaagggcttttctagttttatgagaatttgtactactgatttttatatattcctgtttttgagatgaacagatctctggggaaattgttgagttacaatggcatttcactgtgatccctctcaagctcagatcagttctataacccaatgacaacctgtctctttggtttactgtcctgtgaaatgtcagctcaagtttcccagaagtcgtgtgtttatgatgagtcagagtgcttttcctcggtgggacagttgctggccctcttaattttggtgtatgtgcttccaagtatctaaacctccagtctgatctgtatatgctatcctaactgttaattgtattattgattatgttgattatcttgcttgaaggttcatacttttcaatttgatagaaataaagtttttttctgcttatagctagcgaa

SEQ ID NO: 4 - DNA, 1556 bp, Homo sapiens
gtcgctgcgcggctggccggtgaggccgcggcatggggcgagtgcagctcttcgagatcagcctgagccacggccgcgtcgtctacagccccggggagccgttggctgggaccgtgcgcgtgcgcctgggggcaccgctgccgttccgagccatccgggtgacctgcataggttcctgcggggtctccaacaaggctaatgacacagcgtgggtagtggaggagggttacttcaacagttccctgtcgctggcagacaaggggagcctgcccgctggagagcacagcttccccttccagttcctgcttcctgccactgcacccacgtcctttgagggtcctttcgggaagatcgtgcaccaggtgagggccgccatccacacgccacggttttccaaggatcacaagtgcagcctcgtgttctatatcttgagccccttgaacctgaacagcatcccagacattgagcaacccaacgtggcctctgccaccaagaagttctcctacaagctggtgaagacgggcagcgtggtcctcacagccagcactgatctccgcggctatgtggtggggcaggcactgcagctgcatgccgacgttgagaaccagtcaggcaaggacaccagccctgtggtggccagtctgctgcagaaagtgtcctataaggccaagcgctggatccacgacgtacggaccattgcggaggtggagggtgcgggcgtcaaggcctggcggcgggcgcagtggcacgagcagatcctggtgcctgccttgccccagtcggccctgccgggctgcagcctcatccacatcgactactacttacaggtctctctgaaggcgccggaagctactgtgaccctcccggtcttcattggcaatattgctgtgaaccatgccccagtgagcccccggccaggcctggggctgcctcctggggccccacccctggtggtgccttccgcaccaccccaggaggaggctgaggctgaggctgcggctggcggcccccacttcttggaccccgtcttcctctccaccaagagccattcgcagcggcagcccctgctggccaccttgagttctgtgcctggtgcgccggagccctgccctcaggatggcagccctgcctcacacccgctgcaccctcccttgtgcatttcaacaggtgccactgtcccctactttgcagagggctccggggggccagtgcccactaccagcaccttgattcttcctccagagtacagttcttggggctacccctatgaggccccaccgtcttatgagcagagctgcggcggcgtggaacccagcctgacccctgagagctgaccccgtgctgccttctccaggcaggcctggcctctgccctgggactggggcgcccagggcctcgtgccttctctcttggcctagcctggcccactcaggacctgcccagcctctgccagctcctctggcatccgccctcttctccctggggctggggtgggggtggcagggagctgggacctggagagacaactcctgtaaataaaacactttatttgtaga

SEQ ID NO: 5 - DNA, 1476 bp, Homo sapiens
gccacccaccctccggaccgcggcagctgctgacccgccatcgccatggcccgcgggaaagccaaggaggagggcagctggaagaaattcatctggaactcagagaagaaggagtttctgggcaggaccggtggcagttggtttaagatccttctattctacgtaatattttatggctgcctggctggcatcttcatcggaaccatccaagtgatgctgctcaccatcagtgaatttaagcccacatatcaggaccgagtggccccgccaggattaacacagattcctcagatccagaagactgaaatttcctttcgtcctaatgatcccaagagctatgaggcatatgtactgaacatagttaggttcctggaaaagtacaaagattcagcccagagggatgacatgatttttgaagattgtggcgatgtgcccagtgaaccgaaagaacgaggagactttaatcatgaacgaggagagcgaaaggtctgcagattcaagcttgaatggctgggaaattgctctggattaaatgatgaaacttatggctacaaagagggcaaaccgtgcattattataaagctcaaccgagttctaggcttcaaacctaagcctcccaagaatgagtccttggagacttacccagtgatgaagtataacccaaatgtccttcccgttcagtgcactggcaagcgagatgaagataaggataaagttggaaatgtggagtattttggactgggcaactcccctggttttcctctgcagtattatccgtactatggcaaactcctgcagcccaaatacctgcagcccctgctggccgtacagttcaccaatcttaccatggacactgaaattcgcatagagtgtaaggcgtacggtgagaacattgggtacagtgagaaagaccgttttcagggacgttttgatgtaaaaattaaattttaagtgacactacagaaaaacacaaaaaggtgatgggttgtgttatgcttgtattgaatgctgtcttgacatctcttgccttgtcctccggtatgttctaaagctgtgtctgagatctggatctgcccatcactttggctagtgacagggctaattaatttgctttatacattttcttttactttccttttttcctttctggaggcatcacatgctggtgctgtgtctttatgaatgttttaaccattttcatggtggaagaattttatatttatgcagttgtacaattttatttttttctgcaagaaaaagtgtaatgtatgaaataaaccaaagtcacttgtttgaaaataaatctttattttgaactttataaaaagcaatgcagtaccccatagactggtgttaaatgttgtctacagtgcaaaatccatgttctaacatatgtaataattgccaggagtacagtgctcttgttgatcttgtattcagtcaggttaaaacaacggtcaataaaagaatgaacac

SEQ ID NO: 6 - DNA, 2574 bp, Homo sapiens
ctcgatccgggcgatggaggaggaagcaagcgagggggctggttcctgagcttcgcaattcctgtgtcgccttctgggctcccagcctgccgggtcgcatgatccctccggccggagctggtttttttgccagccaccgcgaggccggctgagttaccggcatccccgcagccacctcctctcccgacctgtgatacaaaagatcttccgggggctgcacctgcctgcctttgcctaaggcggatttgaatctctttctctcccttcagaatcttatcttggctttggatcttagaagagaatcactaaccagagacgagactcagtgagtgagcaggtgttttggacaatggactggttgagcccatccctattataaaaatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaccagacactgaccatccactctaccctcccacccccttctctgctccaccacatcctccgtccagccgccattgccaccaggagaaccactacatgcagcccatgcccacctgcccatcacagggttgggcccagatctggtcccttgcagctagttttctagaatttatcacacttctgtgagacccccacacctcagttcccttggcctcagaattcacaaaatttccacaaaatctgtccaaaggaggctggcaggtatggaagggtttgtggctgggggcaggagggccctacctgattggtgcaacccttaccccttagcctccctgaaaatgtttttctgccagggagcttgaaagttttcagaacctcttccccagaaaggagactagattgcctttgttttgatgtttgtggcctcagaattgatcattttccccccactctccccacactaacctgggttccctttccttccatccctaccccctaagagccatttaggggccacttttgactagggattcaggctgcttgggataaagatgcaaggaccaggactccctcctcacctctggactggctagagtcctcactcccagtccaaatgtcctccagaagcctctggctagaggccagccccacccaggagggagggggctatagctacaggaagcaccccatgccaaagctagggtggcccttgcagttcagcaccaccctagtcccttcccctccctggctcccatgaccatactgagggaccaactgggcccaagacagatgccccagagctgtttatggcctcagctgcctcacttcctacaagagcagcctgtggcatctttgccttgggctgctcctcatggtgggttcaggggactcagccctgaggtgaaagggagctatcaggaacagctatgggagccccagggtcttccctacctcaggcaggaagggcaggaaggagagcctgctgcatggggtggggtagggctgactagaagggccagtcctgcctggccaggcagatctgtgccccatgcctgtccagcctgggcagccaggctgccaaggccagagtggcctggccaggagctcttcaggcctccctctctcttctgctccacccttggcctgtctcatccccaggggtcccagccaccccgggctctctgctgtacatatttgagactagtttttattccttgtgaagatgatatactatttttgttaagcgtgtctgtatttatgtgtgaggagctgctggcttgcagtgcgcgtgcacgtggagagctggtgcccggagattggacggcctgatgctccctcccctgccctggtccagggaagctggccgagggtcctggctcctgaggggcatctgcccctcccccaacccccaccccacacttgttccagctctttgaaatagtctgtgtgaaggtgaaagtgcagttcagtaataaactgtgtttactcagtgaa

SEQ ID NO: 7 - DNA, 3306 bp, Homo sapiens
agaactggggttcttagaagtggtgatgcaagaagtttctaggaaaggccggacaccaggttttgagcaaaattttggactgtgaagcaaggcattggtgaagacaaaatggcctcgccggctgacagctgtatccagttcacccgccatgccagtgatgttcttctcaaccttaatcgtctccggagtcgagacatcttgactgatgttgtcattgttgtgagccgtgagcagtttagagcccataaaacggtcctcatggcctgcagtggcctgttctatagcatctttacagaccagttgaaatgcaaccttagtgtgatcaatctagatcctgagatcaaccctgagggattctgcatcctcctggacttcatgtacacatctcggctcaatttgcgggagggcaacatcatggctgtgatggccacggctatgtacctgcagatggagcatgttgtggacacttgccggaagtttattaaggccagtgaagcagagatggtttctgccatcaagcctcctcgtgaagagttcctcaacagccggatgctgatgccccaagacatcatggcctatcggggtcgtgaggtggtggagaacaacctgccactgaggagcgcccctgggtgtgagagcagagcctttgcccccagcctgtacagtggcctgtccacaccgccagcctcttattccatgtacagccacctccctgtcagcagcctcctcttctccgatgaggagtttcgggatgtccggatgcctgtggccaaccccttccccaaggagcgggcactcccatgtgatagtgccaggccagtccctggtgagtacagccggccgactttggaggtgtcccccaatgtgtgccacagcaatatctattcacccaaggaaacaatcccagaagaggcacgaagtgatatgcactacagtgtggctgagggcctcaaacctgctgccccctcagcccgaaatgccccctacttcccttgtgacaaggccagcaaagaagaagagagaccctcctcggaagatgagattgccctgcatttcgagccccccaatgcacccctgaaccggaagggtctggttagtccacagagcccccagaaatctgactgccagcccaactcgcccacagagtcctgcagcagtaagaatgcctgcatcctccaggcttctggctcccctccagccaagagccccactgaccccaaagcctgcaactggaagaaatacaagttcatcgtgctcaacagcctcaaccagaatgccaaaccagaggggcctgagcaggctgagctgggccgcctttccccacgagcctacacggccccacctgcctgccagccacccatggagcctgagaaccttgacctccagtccccaaccaagctgagtgccagcggggaggactccaccatcccacaagccagccggctcaataacatcgttaacaggtccatgacgggctctccccgcagcagcagcgagagccactcaccactctacatgcaccccccgaagtgcacgtcctgcggctctcagtccccacagcatgcagagatgtgcctccacaccgctggccccacgttccctgaggagatgggagagacccagtctgagtactcagattctagctgtgagaacggggccttcttctgcaatgagtgtgactgccgcttctctgaggaggcctcactcaagaggcacacgctgcagacccacagtgacaaaccctacaagtgtgaccgctgccaggcctccttccgctacaagggcaacctcgccagccacaagaccgtccataccggtgagaaaccctatcgttgcaacatctgtggggcccagttcaaccggccagccaacctgaaaacccacactcgaattcactctggagagaagccctacaaatgcgaaacctgcggagccagatttgtacaggtggcccacctccgtgcccatgtgcttatccacactggtgagaagccctatccctgtgaaatctgtggcacccgtttccggcaccttcagactctgaagagccacctgcgaatccacacaggagagaaaccttaccattgtgagaagtgtaacctgcatttccgtcacaaaagccagctgcgacttcacttgcgccagaagcatggcgccatcaccaacaccaaggtgcaataccgcgtgtcagccactgacctgcctccggagctccccaaagcctgctgaagcatggagtgttgatgctttcgtctccagccccttctcagaatctacccaaaggatactgtaacactttacaatgttcatcccatgatgtagtgcctctttcatccactagtgcaaatcatagctgggggttgggggtggtgggggtcggggcctgggggactgggagccgcagcagctccccctcccccactgccataaaacattaagaaaatcatattgcttcttctcctatgtgtaaggtgaaccatgtcagcaaaaagcaaaatcattttatatgtcaaagcaggggagtatgcaaaagttctgacttgactttagtctgcaaaatgaggaatgtatatgttttgtgggaacagatgtttcttttgtatgtaaatgtgcattcttttaaaagacaagacttcagtatgttgtcaaagagagggctttaatttttttaaccaaaggtgaaggaatatatggcagagttgtaaatatataaatatatatatatataaaataaatatatataaacctaaaaaagatatattaaaaatataaaactgcgttaaaggctcgattttgtatctgcaggcagacacggatctgagaatctttattgagaaagagcacttaagagaatattttaagtattgcatctgtataagtaagaaaatattttgtctaaaatgcctcagtgtatttgtatttttttgcaagtgaaggtttacaatttacaaagtgtgtattaaaaaaaacaaaaagaacaaaaaaatctgcagaaggaaaaatgtgtaattttgttctagttttcagtttgtatatacccgtacaacgtgtcctcacggtgccttttttcacggaagttttcaatgatgggcgagcgtgcaccatccctttttgaagtgtaggcagacacagggacttgaagttgttactaactaaactctctttgggaatgtttgtctcatcccattctgcgtcatgcttgtgttataactactccggagacagggtttggctgtgtctaaactgcattaccgcgttgtaaaatatagctgtacaaatataagaataaaatgttgaaaagtcaaa

SEQ ID NO: 8 - DNA, 6877 bp, Homo sapiens
ggcacggaaaaggccaggcgacaggtgtcgcttgaaaagactgggcttgtccttgctggtgcatgcgtcgtcggcctctgggcagcaggtttacaaaggaggaaaacgacttcttctagatttttttttcagtttcttctataaatcaaaacatctcaaaatggagacctaaaatccttaaagggacttagtctaatctcgggaggtagttttgtgcatgggtaaacaaattaagtattaactggtgttttactatccaaagaatgctaattttataaacatgatcgagttatataaggtataccataatgagtttgattttgaatttgatttgtggaaataaaggaaaagtgattctagctggggcatattgttaaagcatttttttcagagttggccaggcagtctcctactggcacattctcccattatgtagaatagaaatagtacctgtgtttgggaaagattttaaaatgagtgacagttatttggaacaaagagctaataatcaatccactgcaaattaaagaaacatgcagatgaaagttttgacacattaaaatacttctacagtgacaaagaaaaatcaagaacaaagctttttgatatgtgcaacaaatttagaggaagtaaaaagataaatgtgatgattggtcaagaaattatccagttatttacaaggccactgatattttaaacgtccaaaagtttgtttaaatgggctgttaccgctgagaatgatgaggatgagaatgatggttgaaggttacattttaggaaatgaagaaacttagaaaattaatataaagacagtgatgaatacaaagaagatttttataacaatgtgtaaaatttttggccagggaaaggaatattgaagttagatacaattacttacctttgagggaaataattgttggtaatgagatgtgatgtttctcctgccacctggaaacaaagcattgaagtctgcagttgaaaagcccaacgtctgtgagatccaggaaaccatgcttgcaaaccactggtaaaaaaaaaaaaaaaaaaaaaaaaaagccacagtgacttgcttattggtcattgctagtattatcgactcagaacctctttactaatggctagtaaatcataattgagaaattctgaattttgacaaggtctctgctgttgaaatggtaaatttattattttttttgtcatgataaattctggttcaaggtatgctatccatgaaataatttctgaccaaaactaaattgatgcaatttgattatccatcttagcctacagatggcatctggtaacttttgactgttttaaaaaataaatccactatcagagtagatttgatgttggcttcagaaacatttagaaaaacaaaagttcaaaaatgttttcaggaggtgataagttgaataactctacaatgttagttctttgagggggacaaaaaatttaaaatctttgaaaggtcttattttacagccatatctaaattatcttaagaaaatttttaacaaagggaatgaaatatatatcatgattctgtttttccaaaagtaacctgaatatagcaatgaagttcagttttgttattggtagtttgggcagagtctctttttgcagcacctgttgtctaccataattacagaggacatttccatgttctagccaagtatactattagaataaaaaaacttaacattgagttgcttcaacagcatgaaactgagtccaaaagaccaaatgaacaaacacattaatctctgattatttattttaaatagaatatttaattgtgtaagatctaatagtatcattatacttaagcaatcatattcctgatgatctatgggaaataactattatttaattaatattgaaaccaggttttaagatgtgttagccagtcctgttactagtaaatctctttatttggagagaaattttagattgttttgttctccttattagaaggattgtagaaagaaaaaaatgactaattggagaaaaattggggatatatcatatttcactgaattcaaaatgtcttcagttgtaaatcttaccattattttacgtacctctaagaaataaaagtgcttctaattaaaatatgatgtcattaattatgaaatacttcttgataacagaagttttaaaatagccatcttagaatcagtgaaatatggtaatgtattattttcctcctttgagttaggtcttgtgcttttttttcctggccactaaatttcacaatttccaaaaagcaaaataaacatattctgaatatttttgctgtgaaacacttgacagcagagctttccaccatgaaaagaagcttcatgagtcacacattacatctttgggttgattgaatgccactgaaacattctagtagcctggagaagttgacctacctgtggagatgcctgccattaaatggcatcctgatggcttaatacacatcactcttctgtgaagggttttaattttcaacacagcttactctgtagcatcatgtttacattgtatgtataaagattatacaaaggtgcaattgtgtatttcttccttaaaatgtatcagtataggatttagaatctccatgttgaaactctaaatgcatagaaataaaaataataaaaaatttttcattttggcttttcagcctagtattaaaactgataaaagcaaagccatgcacaaaactacctccctagagaaaggctagtcccttttcttccccattcatttcattatgaacatagtagaaaacagcatattcttatcaaatttgatgaaaagcgccaacacgtttgaactgaaatacgacttgtcatgtgaactgtaccgaatgtctacgtattccacttttcctgctggggttcctgtctcagaaaggagtcttgctcgtgctggtttctattacactggtgtgaatgacaaggtcaaatgcttctgttgtggcctgatgctggataactggaaaagaggagacagtcctactgaaaagcataaaaagttgtatcctagctgcagattcgttcagagtctaaattccgttaacaacttggaagctacctctcagcctacttttccttcttcagtaacaaattccacacactcattacttccgggtacagaaaacagtggatatttccgtggctcttattcaaactctccatcaaatcctgtaaactccagagcaaatcaagatttttctgccttgatgagaagttcctaccactgtgcaatgaataacgaaaatgccagattacttacttttcagacatggccattgacttttctgtcgccaacagatctggcaaaagcaggcttttactacataggacctggagacagagtggcttgctttgcctgtggtggaaaattgagcaattgggaaccgaaggataatgctatgtcagaacacctgagacattttcccaaatgcccatttatagaaaatcagcttcaagacacttcaagatacacagtttctaatctgagcatgcagacacatgcagcccgctttaaaacattctttaactggccctctagtgttctagttaatcctgagcagcttgcaagtgcgggtttttattatgtgggtaacagtgatgatgtcaaatgcttttgctgtgatggtggactcaggtgttgggaatctggagatgatccatgggttcaacatgccaagtggtttccaaggtgtgagtacttgataagaattaaaggacaggagttcatccgtcaagttcaagccagttaccctcatctacttgaacagctgctatccacatcagacagcccaggagatgaaaatgcagagtcatcaattatccattttgaacctggagaagaccattcagaagatgcaatcatgatgaatactcctgtgattaatgctgccgtggaaatgggctttagtagaagcctggtaaaacagacagttcagagaaaaatcctagcaactggagagaattatagactagtcaatgatcttgtgttagacttactcaatgcagaagatgaaataagggaagaggagagagaaagagcaactgaggaaaaagaatcaaatgatttattattaatccggaagaatagaatggcactttttcaacatttgacttgtgtaattccaatcctggatagtctactaactgccggaattattaatgaacaagaacatgatgttattaaacagaagacacagacgtctttacaagcaagagaactgattgatacgattttagtaaaaggaaatattgcagccactgtattcagaaactctctgcaagaagctgaagctgtgttatatgagcatttatttgtgcaacaggacataaaatatattcccacagaagatgtttcagatctaccagtggaagaacaattgcggagactacaagaagaaagaacatgtaaagtgtgtatggacaaagaagtgtccatagtgtttattccttgtggtcatctagtagtatgcaaagattgtgctccttctttaagaaagtgtcctatttgtaggagtacaatcaagggtacagttcgtacatttctttcatgaagaagaaccaaaacatcgtctaaactttagaattaatttattaaatgtattataactttaacttttatcctaatttggtttccttaaaatttttatttatttacaactcaaaaaacattgttttgtgtaacatatttatatatgtatctaaaccatatgaacatatattttttagaaactaagagaatgataggcttttgttcttatgaacgaaaaagaggtagcactacaaacacaatattcaatcaaaatttcagcattattgaaattgtaagtgaagtaaaacttaagatatttgagttaacctttaagaattttaaatattttggcattgtactaataccgggaacatgaagccaggtgtggtggtatgtgcctgtagtcccaggctgaggcaagagaattacttgagcccaggagtttgaatccatcctgggcagcatactgagaccctgcctttaaaaacaaacagaacaaaaacaaaacaccagggacacatttctctgtcttttttgatcagtgtcctatacatcgaaggtgtgcatatatgttgaatgacattttagggacatggtgtttttataaagaattctgtgagaaaaaatttaataaagcaacaaaaattactcttattcttcattgctttatttcaatgacattggatagtttagtcactcccagactctttccataccttcttaaagcctctcaaatattgaactacagtttatactccttcccataagatgcttcttcattgacacttgtagaacacggggtcaacacatcataaaatctattatggaatgcctgagacaagaatcaaacagtccctttagtaagtttgtttattcacttctctattgattcattcaagaagtctcatgccagccccacctattggaagaaggtctgagttttattcttatctctttggtattaattctgaaacttagaaagtacactggttagcaatgcttgggaccaacaggttgttctggtaaataaatctgtttcatattgtcagtgcaacaaaatgtccccctctgcattatgttattggtactcaacacgtccgagtcataactctgtcctttgcttcttatagaggtattaggtcttcaagagcagaagtaagactgtaatagggaatactcaggggaaggcaggcaaaggctagtcatctaaaccagttctagatgtctgtataggggcagatggctctgtaagggcagaagggaaagaccccttcataagggtcacagctgacaatcctataacaaaagacaggttaacaagagaaaaacttaacaaatttatttaatcacagatttacatcaccggggagccttcgtaatgaagatccaaaattacaggggaaactgtgcatttttatgcttaggtttgataatgaatggacagccctgaagaatagtgattggaaaaaaaggatatgatctaatgggaatagacacaggttggggacccagcaaggcctgtctgttcagattattcttggtctctgtgcagcattccttcctcctggatatagggcagggcctgtatgggatggggatattataacctgctatcaagcaaggtaggtcagagaatttatttatggccagctcttacatagttaggtgaggaaagattagagtactatctttaagatgtaagtctggcattgtggaaagatggttccagtttctatgacctaccttggggaagaggaattcaagtttctgtggcttgccttcagggagaatgaggctgagacaggagggcaggataacatcagagaaaaactttgcttctgaggccttcactttgggttttctgagccccaacatctgctagtgttgtaaagagaacaattagggaccaagtgaggggaggaaagaatccatctctgcattctgatgctgggagacttatttccttgaaatgcaattgattttgcctctgctaagaggctctgctggctacccatgtactagccagtgtcctgcatgggtgctaggctgaattatttgtaattgtgcttaggtgatttgtaactcaggtatagggtatttaaatagtaggcaccctttttgcaccatgtgtttttttttttatctagttcttgtatactacagataatatttgaactttgtcatctcactgtaaaacttttgttcatttctcattatggtaataaatagctattataaccaacccatttattcaaatatgttatttccctaagtgttattttgacattttgttttggaaaaaataaatcaccatagataataa

SEQ ID NO: 9 - DNA, 4238 bp, Homo sapiens
agagggctgtcggcgcagtagcagcgagcagcagagtccgcacgctccggcgaggggcagaagagcgcgagggagcgcggggcagcagaagcgagagccgagcgcggacccagccaggacccacagccctccccagctgcccaggaagagccccagccatggaacaccagctcctgtgctgcgaagtggaaaccatccgccgcgcgtaccccgatgccaacctcctcaacgaccgggtgctgcgggccatgctgaaggcggaggagacctgcgcgccctcggtgtcctacttcaaatgtgtgcagaaggaggtcctgccgtccatgcggaagatcgtcgccacctggatgctggaggtctgcgaggaacagaagtgcgaggaggaggtcttcccgctggccatgaactacctggaccgcttcctgtcgctggagcccgtgaaaaagagccgcctgcagctgctgggggccacttgcatgttcgtggcctctaagatgaaggagaccatccccctgacggccgagaagctgtgcatctacaccgacaactccatccggcccgaggagctgctgcaaatggagctgctcctggtgaacaagctcaagtggaacctggccgcaatgaccccgcacgatttcattgaacacttcctctccaaaatgccagaggcggaggagaacaaacagatcatccgcaaacacgcgcagaccttcgttgccctctgtgccacagatgtgaagttcatttccaatccgccctccatggtggcagcggggagcgtggtggccgcagtgcaaggcctgaacctgaggagccccaacaacttcctgtcctactaccgcctcacacgcttcctctccagagtgatcaagtgtgacccggactgcctccgggcctgccaggagcagatcgaagccctgctggagtcaagcctgcgccaggcccagcagaacatggaccccaaggccgccgaggaggaggaagaggaggaggaggaggtggacctggcttgcacacccaccgacgtgcgggacgtggacatctgagggcgccaggcaggcgggcgccaccgccacccgcagcgagggcggagccggccccaggtgctcccctgacagtccctcctctccggagcattttgataccagaagggaaagcttcattctccttgttgttggttgttttttcctttgctctttcccccttccatctctgacttaagcaaaagaaaaagattacccaaaaactgtctttaaaagagagagagagaaaaaaaaaatagtatttgcataaccctgagcggtgggggaggagggttgtgctacagatgatagaggattttataccccaataatcaactcgtttttatattaatgtacttgtttctctgttgtaagaataggcattaacacaaaggaggcgtctcgggagaggattaggttccatcctttacgtgtttaaaaaaaagcataaaaacattttaaaaacatagaaaaattcagcaaaccatttttaaagtagaagagggttttaggtagaaaaacatattcttgtgcttttcctgataaagcacagctgtagtggggttctaggcatctctgtactttgcttgctcatatgcatgtagtcactttataagtcattgtatgttattatattccgtaggtagatgtgtaacctcttcaccttattcatggctgaagtcacctcttggttacagtagcgtagcgtgcccgtgtgcatgtcctttgcgcctgtgaccaccaccccaacaaaccatccagtgacaaaccatccagtggaggtttgtcgggcaccagccagcgtagcagggtcgggaaaggccacctgtcccactcctacgatacgctactataaagagaagacgaaatagtgacataatatattctatttttatactcttcctatttttgtagtgacctgtttatgagatgctggttttctacccaacggccctgcagccagctcacgtccaggttcaacccacagctacttggtttgtgttcttcttcatattctaaaaccattccatttccaagcactttcagtccaataggtgtaggaaatagcgctgtttttgttgtgtgtgcagggagggcagttttctaatggaatggtttgggaatatccatgtacttgtttgcaagcaggactttgaggcaagtgtgggccactgtggtggcagtggaggtggggtgtttgggaggctgcgtgccagtcaagaagaaaaaggtttgcattctcacattgccaggatgataagttcctttccttttctttaaagaagttgaagtttaggaatcctttggtgccaactggtgtttgaaagtagggacctcagaggtttacctagagaacaggtggtttttaagggttatcttagatgtttcacaccggaaggtttttaaacactaaaatatataatttatagttaaggctaaaaagtatatttattgcagaggatgttcataaggccagtatgatttataaatgcaatctccccttgatttaaacacacagatacacacacacacacacacacacacaaaccttctgcctttgatgttacagatttaatacagtttatttttaaagatagatccttttataggtgagaaaaaaacaatctggaagaaaaaaaccacacaaagacattgattcagcctgtttggcgtttcccagagtcatctgattggacaggcatgggtgcaaggaaaattagggtactcaacctaagttcggttccgatgaattcttatcccctgccccttcctttaaaaaacttagtgacaaaatagacaatttgcacatcttggctatgtaattcttgtaatttttatttaggaagtgttgaagggaggtggcaagagtgtggaggctgacgtgtgagggaggacaggcgggaggaggtgtgaggaggaggctcccgaggggaaggggcggtgcccacaccggggacaggccgcagctccattttcttattgcgctgctaccgttgacttccaggcacggtttggaaatattcacatcgcttctgtgtatctctttcacattgtttgctgctattggaggatcagttttttgttttacaatgtcatatactgccatgtactagttttagttttctcttagaacattgtattacagatgccttttttgtagttttttttttttttatgtgatcaattttgacttaatgtgattactgctctattccaaaaaggttgctgtttcacaatacctcatgcttcacttagccatggtggacccagcgggcaggttctgcctgctttggcgggcagacacgcgggcgcgatcccacacaggctggcgggggccggccccgaggccgcgtgcgtgagaaccgcgccggtgtccccagagaccaggctgtgtccctcttctcttccctgcgcctgtgatgctgggcacttcatctgatcgggggcgtagcatcatagtagtttttacagctgtgttattctttgcgtgtagctatggaagttgcataattattattattattattataacaagtgtgtcttacgtgccaccacggcgttgtacctgtaggactctcattcgggatgattggaatagcttctggaatttgttcaagttttgggtatgtttaatctgttatgtactagtgttctgtttgttattgttttgttaattacaccataatgctaatttaaagagactccaaatctcaatgaagccagctcacagtgctgtgtgccccggtcacctagcaagctgccgaaccaaaagaatttgcaccccgctgcgggcccacgtggttggggccctgccctggcagggtcatcctgtgctcggaggccatctcgggcacaggcccaccccgccccacccctccagaacacggctcacgcttacctcaaccatcctggctgcggcgtctgtctgaaccacgcgggggccttgagggacgctttgtctgtcgtgatggggcaagggcacaagtcctggatgttgtgtgtatcgagaggccaaaggctggtggcaagtgcacggggcacagcggagtctgtcctgtgacgcgcaagtctgagggtctgggcggcgggcggctgggtctgtgcatttctggttgcaccgcggcgcttcccagcaccaacatgtaaccggcatgtttccagcagaagacaaaaagacaaacatgaaagtctagaaataaaactggtaaaacccca

SEQ ID NO: 10 - DNA, 1607 bp, Homo sapiens
ccgactgaggcacgagcgggtgacgctgggcctgcagcgcggagcagaaagcagaacccgcagagtcctccctgctgctgtgtggacgacacgtgggcacaggcagaagtgggccctgtgaccagctgcactggtttcgtggaaggaagctccaggactggcgggatgggctcagcctgtatcaaagtcaccaaatactttctcttcctcttcaacttgatcttctttatcctgggcgcagtgatcctgggcttcggggtgtggatcctggccgacaagagcagtttcatctctgtcctgcaaacctcctccagctcgcttaggatgggggcctatgtcttcatcggcgtgggggcagtcactatgctcatgggcttcctgggctgcatcggcgccgtcaacgaggtccgctgcctgctggggctgtactttgctttcctgctcctgatcctcattgcccaggtgacggccggggccctcttctacttcaacatgggcaagctgaagcaggagatgggcggcatcgtgactgagctcattcgagactacaacagcagtcgcgaggacagcctgcaggatgcctgggactacgtgcaggctcaggtgaagtgctgcggctgggtcagcttctacaactggacagacaacgctgagctcatgaatcgccctgaggtcacctacccctgttcctgcgaagtcaagggggaagaggacaacagcctttctgtgaggaagggcttctgcgaggcccccggcaacaggacccagagtggcaaccaccctgaggactggcctgtgtaccaggagggctgcatggagaaggtgcaggcgtggctgcaggagaacctgggcatcatcctcggcgtgggcgtgggtgtggccatcatcgagctcctggggatggtcctgtccatctgcttgtgccggcacgtccattccgaagactacagcaaggtccccaagtactgaggcagctgctatccccatctccctgcctggcccccaacctcagggctcccaggggtctccctggctccctcctccaggcctgcctcccacttcactgcgaagaccctcttgcccaccctgactgaaagtagggggctttctggggcctagcgatctctcctggcctatccgctgccagccttgagccctggctgttctgtggttcctctgctcaccgcccatcagggttctcttatcaactcagagaaaaatgctccccacagcgtccctggcgcaggtgggctggacttctacctgccctcaagggtgtgtatattgtataggggcaactgtatgaaaaattggggaggagggggccgggcgcggtgctcacgcctgtaatcccagcactttgggaggccgaggcgggtggatcacgaggtcaggagatcgagaccatcctggctaacatggtgaaaccccgtctctactaaaaatacaaaaaaaatttagccgggcgcggtggcgggcacctgtagtcccagctacttgggaggctgaggcaggagaatggtgtgaacccgggagcggaggttgcagtgagctgagatcgtgctactgcactccagcctgggggacagaaagagactccgtctcaa

SEQ ID NO: 11 - DNA, 2122 bp, Homo sapiens
ggtggctattttgtccttgggctgcctgttttcagctgctgcaaccacagggatttcttctgttcaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttacttgtaaaaaaaaaaaaaaaaaa

SEQ ID NO: 12 - DNA, 1744 bp, Homo sapiens
gcagcaggccaagggggaggtgcgagcgtggacctgggacgggtctgggcggctctcggtggttggcacgggttcgcacacccattcaagcggcaggacgcacttgtcttagcagttctcgctgaccgcgctagctgcggcttctacgctccggcactctgagttcatcagcaaacgccctggcgtctgtcctcaccatgcctagcctttgggaccgcttctcgtcgtcgtccacctcctcttcgccctcgtccttgccccgaactcccaccccagatcggccgccgcgctcagcctgggggtcggcgacccgggaggaggggtttgaccgctccacgagcctggagagctcggactgcgagtccctggacagcagcaacagtggcttcgggccggaggaagacacggcttacctggatggggtgtcgttgcccgacttcgagctgctcagtgaccctgaggatgaacacttgtgtgccaacctgatgcagctgctgcaggagagcctggcccaggcgcggctgggctctcgacgccctgcgcgcctgctgatgcctagccagttggtaagccaggtgggcaaagaactactgcgcctggcctacagcgagccgtgcggcctgcggggggcgctgctggacgtctgcgtggagcagggcaagagctgccacagcgtgggccagctggcactcgaccccagcctggtgcccaccttccagctgaccctcgtgctgcgcctggactcacgactctggcccaagatccaggggctgtttagctccgccaactctcccttcctccctggcttcagccagtccctgacgctgagcactggcttccgagtcatcaagaagaagctgtacagctcggaacagctgctcattgaggagtgttgaacttcaacctgagggggccgacagtgccctccaagacagagacgactgaacttttggggtggagactagaggcaggagctgagggactgattcctgtggttggaaaactgaggcagccacctaaggtggaggtgggggaatagtgtttcccaggaagctcattgagttgtgtgcgggtggctgtgcattggggacacatacccctcagtactgtagcatgaaacaaaggcttaggggccaacaaggcttccagctggatgtgtgtgtagcatgtaccttattatttttgttactgacagttaacagtggtgtgacatccagagagcagctgggctgctcccgccccagcccggcccagggtgaaggaagaggcacgtgctcctcagagcagccggagggaggggggaggtcggaggtcgtggaggtggtttgtgtatcttactggtctgaagggaccaagtgtgtttgttgtttgttttgtatcttgtttttctgatcggagcatcactactgacctgttgtaggcagctatcttacagacgcatgaatgtaagagtaggaaggggtgggtgtcagggatcacttgggatctttgacacttgaaaaattacacctggcagctgcgtttaagccttcccccatcgtgtactgcagagttgagctggcaggggaggggctgagagggtgggggctggaacccctccccgggaggagtgccatctgggtcttccatctagaactgtttacatgaagataagatactcactgttcatgaatacacttgatgttcaagtattaagacctatgcaatattttttacttttctaataaacatgtttgttaaaaca

SEQ ID NO: 13 - DNA, 2690 bp, Homo sapiens
gggactttgcaggcagcggcggccgggggcggagcgggatcgagccctcgccgaggcctgccgccatgggcccgcgccgccgccgccgcctgtcacccgggccgcgcgggccgtgagcgtcatggccttggccggggcccctgcgggcggcccatgcgcgccggcgctggaggccctgctcggggccggcgcgctgcggctgctcgactcctcgcagatcgtcatcatctccgccgcgcaggacgccagcgccccgccggctcccaccggccccgcggcgcccgccgccggcccctgcgaccctgacctgctgctcttcgccacaccgcaggcgccccggcccacacccagtgcgccgcggcccgcgctcggccgcccgccggtgaagcggaggctggacctggaaactgaccatcagtacctggccgagagcagtgggccagctcggggcagaggccgccatccaggaaaaggtgtgaaatccccgggggagaagtcacgctatgagacctcactgaatctgaccaccaagcgcttcctggagctgctgagccactcggctgacggtgtcgtcgacctgaactgggctgccgaggtgctgaaggtgcagaagcggcgcatctatgacatcaccaacgtccttgagggcatccagctcattgccaagaagtccaagaaccacatccagtggctgggcagccacaccacagtgggcgtcggcggacggcttgaggggttgacccaggacctccgacagctgcaggagagcgagcagcagctggaccacctgatgaatatctgtactacgcagctgcgcctgctctccgaggacactgacagccagcgcctggcctacgtgacgtgtcaggaccttcgtagcattgcagaccctgcagagcagatggttatggtgatcaaagcccctcctgagacccagctccaagccgtggactcttcggagaactttcagatctcccttaagagcaaacaaggcccgatcgatgttttcctgtgccctgaggagaccgtaggtgggatcagccctgggaagaccccatcccaggaggtcacttctgaggaggagaacagggccactgactctgccaccatagtgtcaccaccaccatcatctcccccctcatccctcaccacagatcccagccagtctctactcagcctggagcaagaaccgctgttgtcccggatgggcagcctgcgggctcccgtggacgaggaccgcctgtccccgctggtggcggccgactcgctcctggagcatgtgcgggaggacttctccggcctcctccctgaggagttcatcagcctttccccaccccacgaggccctcgactaccacttcggcctcgaggagggcgagggcatcagagacctcttcgactgtgactttggggacctcacccccctggatttctgacagggcttggagggaccagggtttccagagatgctcaccttgtctctgcagccctggagccccctgtccctggccgtcctcccagcctgtttggaaacatttaatttatacccctctcctctgtctccagaagcttctagctctggggtctggctaccgctaggaggctgagcaagccaggaagggaaggagtctgtgtggtgtgtatgtgcatgcagcctacacccacacgtgtgtaccgggggtgaatgtgtgtgagcatgtgtgtgtgcatgtaccggggaatgaaggtgaacatacacctctgtgtgtgcactgcagacacgccccagtgtgtccacatgtgtgtgcatgagtccatgtgtgcgcgtgggggggctctaactgcactttcggcccttttgctctgggggtcccacaaggcccagggcagtgcctgctcccagaatctggtgctctgaccaggccaggtggggaggctttggctggctgggcgtgtaggacggtgagagcacttctgtcttaaaggttttttctgattgaagctttaatggagcgttatttatttatcgaggcctctttggtgagcctggggaatcagcaaaggggaggaggggtgtggggttgataccccaactccctctacccttgagcaagggcaggggtccctgagctgttcttctgccccatactgaaggaactgaggcctgggtgatttatttattgggaaagtgagggagggagacagactgactgacagccatgggtggtcagatggtggggtgggccctctccagggggccagttcagggccccagctgccccccaggatggatatgagatgggagaggtgagtgggggaccttcactgatgtgggcaggaggggtggtgaaggcctcccccagcccagaccctgtggtccctcctgcagtgtctgaagcgcctgcctccccactgctctgccccaccctccaatctgcactttgatttgcttcctaacagctctgttccctcctgctttggttttaataaatattttgatgacgtttgggccgggttttgggactctgttgggaacatttcggggcgggagaggccaaggttgctggggaaatgcccattctccacttcccttctccctgtccgtgcccgatttgatttgagcctcataactcgaagaaaggtcagcttcctcgctgttttggtcctaactcaaaagcagatccagtaaaggtttttgttgtagaaagccaa

SEQ ID NO: 14 - DNA, 2298 bp, Homo sapiens
aagactgcgagctccccgcaccccctcgcactccctctggccggcccagggcgccttcagcccaacctccccagccccacgggcgccacggaacccgctcgatctcgccgccaactggtagacatggagacccctgcctggccccgggtcccgcgccccgagaccgccgtcgctcggacgctcctgctcggctgggtcttcgcccaggtggccggcgcttcaggcactacaaatactgtggcagcatataatttaacttggaaatcaactaatttcaagacaattttggagtgggaacccaaacccgtcaatcaagtctacactgttcaaataagcactaagtcaggagattggaaaagcaaatgcttttacacaacagacacagagtgtgacctcaccgacgagattgtgaaggatgtgaagcagacgtacttggcacgggtcttctcctacccggcagggaatgtggagagcaccggttctgctggggagcctctgtatgagaactccccagagttcacaccttacctggagacaaacctcggacagccaacaattcagagttttgaacaggtgggaacaaaagtgaatgtgaccgtagaagatgaacggactttagtcagaaggaacaacactttcctaagcctccgggatgtttttggcaaggacttaatttatacactttattattggaaatcttcaagttcaggaaagaaaacagccaaaacaaacactaatgagtttttgattgatgtggataaaggagaaaactactgtttcagtgttcaagcagtgattccctcccgaacagttaaccggaagagtacagacagcccggtagagtgtatgggccaggagaaaggggaattcagagaaatattctacatcattggagctgtggtatttgtggtcatcatccttgtcatcatcctggctatatctctacacaagtgtagaaaggcaggagtggggcagagctggaaggagaactccccactgaatgtttcataaaggaagcactgttggagctactgcaaatgctatattgcactgtgaccgagaacttttaagaggatagaatacatggaaacgcaaatgagtatttcggagcatgaagaccctggagttcaaaaaactcttgatatgacctgttattaccattagcattctggttttgacatcagcattagtcactttgaaatgtaacgaatggtactacaaccaattccaagttttaatttttaacaccatggcaccttttgcacataacatgctttagattatatattccgcactcaaggagtaaccaggtcgtccaagcaaaaacaaatgggaaaatgtcttaaaaaatcctgggtggacttttgaaaagcttttttttttttttttttttttttgagacggagtcttgctctgttgcccaggctggagtgcagtagcacgatctcggctcactgcaccctccgtctctcgggttcaagcaattgtctgcctcagcctcccgagtagctgggattacaggtgcgcactaccacgccaagctaatttttgtattttttagtagagatggggtttcaccatcttggccaggctggtcttgaattcctgacctcaggtgatccacccaccttggcctcccaaagtgctagtattatgggcgtgaaccaccatgcccagccgaaaagcttttgaggggctgacttcaatccatgtaggaaagtaaaatggaaggaaattgggtgcatttctaggacttttctaacatatgtctataatatagtgtttaggttcttttttttttcaggaatacatttggaaattcaaaacaattggcaaactttgtattaatgtgttaagtgcaggagacattggtattctgggcaccttcctaatatgctttacaatctgcactttaactgacttaagtggcattaaacatttgagagctaactatatttttataagactactatacaaactacagagtttatgatttaaggtacttaaagcttctatggttgacattgtatatataattttttaaaaaggttttctatatggggattttctatttatgtaggtaatattgttctatttgtatatattgagataatttatttaatatactttaaataaaggtgactgggaattgttactgttgtacttattctatcttccatttattatttatgtacaatttggtgtttgtattagctctactacagtaaatgactgtaaaattgtcagtggcttacaacaacgtatctttttcgcttataatacattttggtgactgta

SEQ ID NO: 15 - DNA, 2291 bp, Homo sapiens
gaacaatgaagaaagccccacagccactgttgctgagcagggagaggatattacctccaaaaaagacaggggagtattaaagattgtcaaaagagtggggaatggtgaggaaacgccgatgattggagacaaagtttatgtccattacaaaggaaaattgtcaaatggaaagaagtttgattccagtcatgatagaaatgaaccatttgtctttagtcttggcaaaggccaagtcatcaaggcatgggacattggggtggctaccatgaagaaaggagagatatgccatttactgtgcaaaccagaatatgcatatggctcggctggcagtctccctaaaattccctcgaatgcaactctcttttttgagattgagctccttgatttcaaaggagaggatttatttgaagatggaggcattatccggagaaccaaacggaaaggagagggatattcaaatccaaacgaaggagcaacagtagaaatccacctggaaggccgctgtggtggaaggatgtttgactgcagagatgtggcattcactgtgggcgaaggagaagaccacgacattccaattggaattgacaaagctctggagaaaatgcagcgggaagaacaatgtattttatatcttggaccaagatatggttttggagaggcagggaagcctaaatttggcattgaacctaatgctgagcttatatatgaagttacacttaagagcttcgaaaaggccaaagaatcctgggagatggataccaaagaaaaattggagcaggctgccattgtcaaagagaagggaaccgtatacttcaagggaggcaaatacatgcaggcggtgattcagtatgggaagatagtgtcctggttagagatggaatatggtttatcagaaaaggaatcgaaagcttctgaatcatttctccttgctgcctttctgaacctggccatgtgctacctgaagcttagagaatacaccaaagctgttgaatgctgtgacaaggcccttggactggacagtgccaatgagaaaggcttgtataggaggggtgaagcccagctgctcatgaacgagtttgagtcagccaagggtgactttgagaaagtgctggaagtaaacccccagaataaggctgcaagactgcagatctccatgtgccagaaaaaggccaaggagcacaacgagcgggaccgcaggatatacgccaacatgttcaagaagtttgcagagcaggatgccaaggaagaggccaataaagcaatgggcaagaagacttcagaaggggtcactaatgaaaaaggaacagacagtcaagcaatggaagaagagaaacctgagggccacgtatgacgccacgccaaggagggaagagtcccagtgaactcggcccctcctcaatgggctttcccccaactcaggacagaacagtgtttaatgtaaagtttgttatagtctatgtgattctggaagcaaatggcaaaaccagtagcttcccaaaaacagcccccctgctgctgcccggagggttcactgaggggtggcacgggaccactccaggtggaacaaacagaaatgactgtggtgtggagggagtgagccagcagcttaagtccagctcatttcagtttctatcaaccttcaagtatccaattcagggtccctggagatcatcctaacaatgtggggctgttaggttttacctttgaactttcatagcactgcagaaaccttttaaaaaaaaatgcttcatgaatttctcctttcctacagttgggtagggtaggggaaggaggataagcttttgttttttaaatgactgaagtgctataaatgtagtctgttgcatttttaaccaacagaacccacagtagaggggtctcatgtctccccagttccacagcagtgtcacagacgtgaaagccagaacctcagaggccacttgcttgctgacttagcctcctcccaaagtccccctcctcagccagcctccttgtgagagtggctttctaccacacacagcctgtccctgggggagtaattctgtcattcctaaaacacccttcagcaatgataatgagcagatgagagtttctggattagcttttcctattttcgatgaagttctgagatactgaaatgtgaaaagagcaatcagaattgtgctttttctcccctcctctattccttttagggaataatattcaatacacagtacttcctcccagaaaaaaaaaaaaaaaaaaaaaaaaaaaaaaaaaaaaaaaaaaaaaaaaaaaaaaaaaaaaaaaaa

SEQ ID NO: 16 - DNA, 1978 bp, Homo sapiens
agacctgacctgccagctccgggcgtggggtgaaatctcttgattcctagtctctcgatatggcacctccgtcagtctttgccgaggttccgcaggcccagcctgtcctggtcttcaagctcactgccgacttcagggaggatccggacccccgcaaggtcaacctgggagtgggagcatatcgcacggatgactgccatccctgggttttgccagtagtgaagaaagtggagcagaagattgctaatgacaatagcctaaatcacgagtatctgccaatcctgggcctggctgagttccggagctgtgcttctcgtcttgcccttggggatgacagcccagcactcaaggagaagcgggtaggaggtgtgcaatctttggggggaacaggtgcacttcgaattggagctgatttcttagcgcgttggtacaatggaacaaacaacaagaacacacctgtctatgtgtcctcaccaacctgggagaatcacaatgctgtgttttccgctgctggttttaaagacattcggtcctatcgctactgggatgcagagaagagaggattggacctccagggcttcctgaatgatctggagaatgctcctgagttctccattgttgtcctccacgcctgtgcacacaacccaactgggattgacccaactccggagcagtggaagcagattgcttctgtcatgaagcaccggtttctgttccccttctttgactcagcctatcagggcttcgcatctggaaacctggagagagatgcctgggccattcgctattttgtgtctgaaggcttcgagttcttctgtgcccagtccttctccaagaacttcgggctctacaatgagagagtcgggaatctgactgtggttggaaaagaacctgagagcatcctgcaagtcctttcccagatggagaagatcgtgcggattacttggtccaatccccccgcccagggagcacgaattgtggccagcaccctctctaaccctgagctctttgaggaatggacaggtaatgtgaagacaatggctgaccggattctgaccatgagatctgaactcagggcacgactagaagccctcaaaacccctgggacctggaaccacatcactgatcaaattggcatgttcagcttcactgggttgaaccccaagcaggttgagtatctggtcaatgaaaagcacatctacctgctgccaagtggtcgaatcaacgtgagtggcttaaccaccaaaaatctagattacgtggccacctccatccatgaagcagtcaccaaaatccagtgaagaaacaccacccgtccagtaccaccaaagtagttctctgtcatgtgtgttccctgcctgcacaaacctacatgtacataccatggattagagacacttgcaggactgaaaggctgctctggtgaggcagcctctgtttaaaccggccccacatgaagagaacatcccttgagacgaatttggagactgggattagagcctttggaggtcaaagcaaattaagatttttatttaagaataaaagagtactttgatcatgagacataggtatcttgtccctctcactaaaaaggagtgttgtgtgtggcggccacgtgcttctatgtggtgtttgactctgtacaaattctagtcccaaagatcaagttgtctgaaggagccaaagtgtgaatgtgggtgtcggctgcggcattaaattcatcatctcaacccagagtgtctggtctccctgctctttctgcatggttgtgtccctagtcctaagctttggttctttagggtgactgtggtaagaaggatatttaatcatgacatgcacggacacgtacatatttaactgaaacaagttttaccaaacagtatttactcgtgatgtgcgtagtgcattctgatatttttgagccattctattgtgttctacttcacctaaaaaaataaaataaaaatgttgatcaaga

SEQ ID NO: 17 - DNA, 4562 bp, Homo sapiens
aaatgtaatttgaagaaggcagaaggaacccatggctttagccggctgcccagattcctttttgcaccatccgtactaccaggacaaggtggagcagacacctcgcagtcaacaagacccggcaggaccaggactccccgcacagtctgaccgacttgcgaatcaccaggaggatgatgtggacctggaagccctggtgaacgatatgaatgcatccctggagagcctgtactcggcctgcagcatgcagtcagacacggtgcccctcctgcagaatggccagcatgcccgcagccagcctcgggcttcaggccctcctcggtccatccagccacaggtgtccccgaggcagagggtgcagcgctcccagcctgtgcacatcctcgctgtcaggcgccttcaggaggaagaccagcagtttagaacctcatctctgccggccatccccaatccttttcctgaactctgtggccctgggagcccccctgtgctcacgccgggttctttacctccgagccaggccgccgcaaagcaggatgttaaagtctttagtgaagatgggacaagcaaagtggtggagattctagcagacatgacagccagagacctgtgccaattgctggtttacaaaagtcactgtgtggatgacaacagctggacactagtggagcaccacccgcacctaggattagagaggtgcttggaagaccatgagctggtggtccaggtggagagtaccatggccagtgagagtaaatttctattcaggaagaattacgcaaaatacgagttctttaaaaatcccatgaatttcttcccagaacagatggttacttggtgccagcagtcaaatggcagtcaaacccagcttttgcaggaacccagacacctgcagctgctggccgacctggaggacagcaacatcttctccctgatcgctggcaggaagcagtacaacgcccctacagaccacgggctctgcataaagccaaacaaagtcaggaatgaaactaaagagctgaggttgctctgtgcagaggacgagcaaaccaggacgtgctggatgacagcgttcagactcctcaagtatggaatgctcctttaccagaattaccgaatccctcagcagaggaaggccttgctgtccccgttctcgacgccagtgcgcagtgtctccgagaactccctcgtggcaatggatttttctgggcaaacaggacgcgtgatagagaatccggcagaggcccagagcgcagccctggaggagggccacgcctggaggaagcgaagcacacggatgaacatcctaggtagccaaagtcccctccacccttctaccctaagtacagtgattcacaggacacagcactggtttcacgggaggatctccagggaggaatcccacaggatcattaaacagcaagggctcgtggatgggctttttctcctccgtgacagccagagtaatccaaaggcatttgtactcacactgtgtcatcaccagaaaattaaaaatttccagatcttaccttgcgaggacgacgggcagacgttcttcagcctagatgacgggaacaccaaattctctgacctgatccagctggttgacttttaccagctgaacaaaggagtcctgccttgcaaactcaagcaccactgcatccgagtggccttatgaccgcagatgtcctctcggctgaagactggaggaagtgaacactggagtgaagaagcggtctgtgcgttggtgaagaacacacatcgattctgcacctggggacccagagcgagatgggtttgttcggtgccagccgaccaagattgactagtttgttggacttaaacgacgatttgctgctgtgaacccagcagggtcgcctccctctgcatcggccaaattggggagggcatggaagatccagcggaaagttgaaaataaactggaatgatcatcttggcttgggccgcttaggaacaagaaccggagagaagtgattggaaatgaactcttgccctggaataatcttgacaattaaaactgatatgtttactttttttgtattgatcacttttttgcactccttctttgttttcaatattgtattcagcctattgtaggagggggatgtggcgtttcaactcatataatacagaaagagttttgaatgggcagatttcaaactgaatatgggtccccaaatgttcccagagggtcctccacaccctctgccgactaccacggtgtggattcagctcccaaatgacaaacccagcccttcccagtatacttgaaaagctttcttgttaaaataaaaggtgtcactgtggtaggcatttggcatattttgtggactcagtcaagcaaccacagtctgttaatcatttctctatgctcagatgtcagatcctcttgttattagtgtgtcttgttctgcacagtgcaggagactttattcctttggaaaattcactgttccacaaacagcaggctgaatggcctcgcctctagattgacgtgggccagcctccttgagacacacctggcacccgtcatcggccagcggtggatgctgcataatccacctgggtacttcagccttgcgtttccacagccttcagcctgttctagaacgatcactgccttacccctgctgctgcagtggtgtgagtcgtttcacggctgatgtccctcgggggattaaaggatctaaagagaaaatggcacctggttgtcttcgtgctgtgtctcatgggtttccatagtgataaagacaaggaaacgctgcaggggccacaggcacaggctgatatttaaagatctttgcttgcagccctccgtcctgctgaaaacccccataagccagtgaacacagagcagctagaggctcctcctctgctggcttagggtcagaagtacctcacagtggttgtggacatggaagagttttgtcaacacaacactttgtccccgctccgggagatgagtcagatggtggcttgagttgtcacttggtcccctccgcccctcgggtggccccctttgccacgtccccttagcttagtgatcaggtgtgagagtggccatttccttacctttgatccctgtaaagcagaaaggactcctttgacaggcgacaaactactgtggtgagcagaatgatttcctttttcaagacaacacctgcctggcttctattaatgtgtgctggccatgatattgccccaaatccgccccactgaagtgttccctaaggaacagcatttctctgctcctcagtcaacccccgtagcctagagcagtgtcacaagcttcagtaaggccagtcagctggaagtcagtctaccgtatagtaacactgtatgtcagtctacagaccacactctagttgttttccatgaaaggtatacaaatgaagaattttctagcaaaacatgtttttaaccatcagtgctcaattgcattttcttcctttcgcagccagtcagtctttcaaactattgacagtaagataattctcacgttcacacctggtggcaggcttcactgtagggacggacattgcagttacaccacgattccttcctcttcactggctcgaggtaaacccttttcaaggaaaaacaactctaggatttcttttttctgtgtacgtagaccagtcccatcagtgtataatctctctctcacacgcctctctccaatagacagcttgtatttgcagtatttcatatttataaatatgcgtttatttaaaaggagaacaaaagcttgactctgattcacagttttgtatgtagctggtttgacgtagtcttttgtattttccctgccgaagtgaattgttggagaatgtaaaccgcctccacgtggcggcagacttcctaaggccccagctcgctggcctcgcgctgggcggctgggaattccacctgagaacaagtcccgcaaaccggggacggaaggacatttgacttttatttttgtatttaattgacatgaatgtaaaggggacagctcagggttgttttggagcctgttgactttgtatctctgcctgtgattttcttttctaaatgaaactccatgtagcaaccaggacgaagttgagaaggaaaacgccaaatgctttggttattagagtttaataggtaagctctgttacactaggtgttagagttccagaatgttcttttgtttgctaaaccttgaagaaacatgtgcctcagcctagatgttttgtcttctcttttctgcacttaatacctgacagtatgaccgatctctgcgcctttctgggggcgggcaagctggcggtagatttgtgatgtcacagtgcaaactgcagtgactgtaaattggcctggcgtgtataaacgttttcagggaatgcagaaggtattaatgaagagacaaaacctttattccatgtgctttgcttcattctgtacatagctctttggctcgtgaacctaattgtaaactttcaggtatttttgtacaaataagggactgatgttctgtttcttgtaattagaaataaacattaatacagtgttcttca

SEQ ID NO: 18 - DNA, 1749 bp, Homo sapiens
aggcgggccgcggcggcggcgggcagcggacgggcggactgacgggcgcctccaccttgctccctccctggctgccggcttccttttgtctttctgggcggcgatgagcgcagggccggcgcagcagctgcgggcgcacggaggcccgcgctgctagtcactcctcccggcctcggcgcgcttgtcccgggcagcggcccgggcccgctgcagccgccgccggcgccgaacttgggctcgggaagccggcggaccgcgtcctgcgccggagcaggggcatggtctagtggcccagtcaggacgcggaaacactccctggaggttctgacccactccctctcagcctccgcctggtctctggtgtagtcgccgccgccagccgccatgggcaaacagaacagcaagctgcggcccgaggtgctgcaggacctgcgggagaacacggagttcaccgaccacgagctgcaggagtggtacaagggcttcctcaaggactgccccaccggccacctgaccgtggacgagttcaagaagatctacgccaacttcttcccctacggcgacgcttccaagttcgccgagcacgtcttccgcaccttcgacaccaacggcgacggcaccatcgacttccgggagttcatcattgcgctgagcgtgacctcgcggggcaagctggagcagaagctcaagtgggccttcagcatgtacgacctggacggcaacggctacatcagccgcagcgagatgctggagatcgtgcaggccatctacaagatggtgtcgtctgtgatgaagatgccggaggatgagtccaccccggagaagcgcacagacaagatcttcaggcagatggacaccaacaatgacggcaaactgtccttggaagaattcatcagaggtgccaagagcgacccctccatcgtccgcctgctgcagtgcgaccccagcagtgccagtcagttctgagcgagcggcccctggacagttgcagagaaacacaggcttgtcgtgccgtttaagctttgcttgcaagagtggatgccccgcaatcgttcctgctctcccgggccccgggcctggggcatgcgttgcacctgcccagcccggtggctgcgcctccctcctccacctgaccaacgcgacattcctcccctcacgcctggcccggtcccttccagggcaactcccagggatgtggtgacatgcagggttcaagtgttcttggttccaggcacctcccggctcacggggagctcagaggtccatgccgaggagaccaggcaggacctcccgaggctgcgccccggccggcccatgcgttttgtgatcccaagtgactctgtgggaagggtggggacgaggcgtcgggagggtatacagggagcccctcccgtgcatggctgcccccccgttcattttctccaccacagccgcttgcacgtatagatactgtggtcccctttcttttaatatataaattatgtatggtgaagtggagtgtattgtgtaggtcccgtatttaatgcctctgactgcctttgaagcgcagccctctgtggcccgcagccccctgagcctggctgttgtgtggtatttatgctctctttgtctgcctgtttctaaggaaatgcatgtgtgccctgagccgtgatgatcctcccatccgtgttgtgagcacaggcatttgtgtctggtctgtcctccctgttgattggtctggcatttccggtattaaaatgataaaataaa

SEQ ID NO: 19 - DNA, 1896 bp, Homo sapiens
gaaagcgagtgtccctctcgcgccccaggccggtgtacccccgcactccgcgccccggcctagaagctctctctccccgctccccggcccggcccccgccccgccccgccccagcccgctgggccgccatggagcgctggccttggccgtcgggcggcgcctggctgctcgtggctgcccgcgcgctgctgcagctgctgcgctcagacctgcgtctgggccgcccgctgctggcggcgctggcgctgctggccgcgctcgactggctgtgccagcgcctgctgcccccgccggccgcactcgccgtgctggccgccgccggctggatcgcgttgtcccgcctggcgcgcccgcagcgcctgccggtggccactcgcgcggtgctcatcaccggctgtgactctggttttggcaaggagacggccaagaaactggactccatgggcttcacggtgctggccaccgtattggagttgaacagccccggtgccatcgagctgcgtacctgctgctcccctcgcctaaggctgctgcagatggacctgaccaaaccaggagacattagccgcgtgctagagttcaccaaggcccacaccaccagcaccggcctgtggggcctcgtcaacaacgcaggccacaatgaagtagttgctgatgcggagctgtctccagtggccactttccgtagctgcatggaggtgaatttctttggcgcgctcgagctgaccaagggcctcctgcccctgctgcgcagctcaaggggccgcatcgtgactgtggggagcccagcgggggacatgccatatccgtgcttgggggcctatggaacctccaaagcggccgtggcgctactcatggacacattcagctgtgaactccttccctggggggtcaaggtcagcatcatccagcctggctgcttcaagacagagtcagtgagaaacgtgggtcagtgggaaaagcgcaagcaattgctgctggccaacctgcctcaagagctgctgcaggcctacggcaaggactacatcgagcacttgcatgggcagttcctgcactcgctacgcctggccatgtccgacctcaccccagttgtagatgccatcacagatgcgctgctggcagctcggccccgccgccgctattaccccggccagggcctggggctcatgtacttcatccactactacctgcctgaaggcctgcggcgccgcttcctgcaggccttcttcatcagtcactgtctgcctcgagcactgcagcctggccagcctggcactaccccaccacaggacgcagcccaggacccaaacctgagccccggcccttccccagcagtggctcggtgagccatgtgcacctatggcccagccactgcagcacaggaggctccgtgagcccttggttcctccccgaaaacccccagcattacgatcccccaagtgtcctggaccctggcctaaagaatcccacccccacttcatgcccactgccgatgcccaatccaggcccggtgaggccaaggtttcccagtgagcctctgcgcctctccactgtttcatgagcccaaacaccctcctggcacaacgctctaccctgcagcttggagaactccgctggatggggagtctcatgcaagacttcactgcagcctttcacaggactctgcagatagtgcctctgcaaactaaggagtgactaggtgggttggggaccccctcaggattgtttctcggcaccagtgcctcagtgctgcaattgagggctaaatcccaagtgtctcttgactggctcaagaattagggccccaactacacacccccaagccacagggaagcatgtactgtacttcccaattgccacattttaaataaagacaaatttttatttcttctaaaa

SEQ ID NO: 20 - DNA, 5213 bp, Homo sapiens
acagctgtcgcccaccgcgggtgagaggcttgctggtccgccccggcttccccgccccggcggcggccgtgcggttcgggctttaattctggcgcttgccagccggggccgactccgccccctgcgccccgcccttccccgagctccgggtccgcggcggagcgagcgagcggccggcaggttgggaggagcggccgaaggttgaatgcctttgagaacttgatgcataaaatttgcatgactcctcactcctttctggatctctcattggactcaagccagcatggctcacaccacaaaggttaacggcagtgcctcaggaaaagcaggtgatattctcagtggagaccaggacaaggaacagaaagacccttactttgtggagaccccctatggttatcaactagacttagatttcctcaaatatgtggatgacatacagaagggaaataccatcaaaagactgaacatccagaagaggcggaagccgtccgtgccatgcccagaacccaggaccacatctggtcagcaaggtatatggacttccactgaatccctctcatcctccaacagtgatgacaacaagcagtgccccaacttcctcatagccagaagtcaagttacatcaactccaatctcaaagccacctccccctctggagacctcactcccttttcttaccatcccagaaaatcgacagctgccacctccctcaccacaactcccaaagcataaccttcatgtcaccaagacactgatggagacccggagaagactggaacaggagagagccaccatgcagatgacaccgggtgagttcagaaggcccaggctggccagttttggaggcatgggcaccacaagctccctcccttcttttgtgggttctggaaaccacaatcctgccaagcaccagcttcagaatggataccaaggtaatggggattatggtagctatgccccagctgctcccaccacttcctccatggggagctccatccgccacagccccctgagctcagggatctccaccccagtgaccaacgtgagccccatgcacctgcagcacatccgcgagcagatggccattgctctgaaacgcctgaaggagctggaggagcaggtgcgaaccatccctgtgctccaggtaaagatctctgtcttgcaagaagagaaaaggcagttggtctcacagctgaaaaaccaaagggctgcatcccagatcaatgtctgtggtgtgaggaagcggtcctatagtgcggggaacgcctcccagctggaacagctctcccgggcccgaagaagtggcggggaattatacattgactatgaggaggaagaaatggagaccgtagaacagagcacgcagaggataaaggagttccggcaacttacagcagacatgcaagccctggagcagaagatccaggacagcagctgtgaggcctcctcagagctcagggagaatggagagtgccggtctgtggctgtgggtgccgaggagaacatgaacgacatcgtcgtgtaccacagaggctccaggtcctgtaaggatgcagctgtagggacacttgttgagatgagaaattgtggggtcagcgtgacagaggccatgcttggagtgatgactgaagctgacaaagaaattgagctgcaacagcagaccatagaatccttgaaggaaaagatctatcgcctagaagtacagcttagagaaaccacccatgaccgggagatgactaaactgaaacaagagctgcaggctgctggatcgaggaaaaaggttgacaaagccacgatggcccagccgcttgttttcagtaaggtggtggaggcagtggtgcagaccagagaccaaatggtcggcagtcacatggacctggtggacacgtgtgttgggacctccgtggaaacaaacagtgtaggcatctcctgccagcctgaatgtaagaataaagtcgtagggcctgagctgcctatgaattggtggattgttaaggagagggtggaaatgcatgaccgatgtgctgggaggtctgtggaaatgtgtgacaagagtgtgagtgtggaagtcagcgtctgcgaaacaggcagcaacacagaggagtctgtgaacgacctcacactcctcaagacaaacttgaatctcaaagaagtgcggtctatcggttgtggagattgttctgttgacgtgaccgtctgctctccaaaggagtgcgcctcccggggcgtgaacactgaggctgttagccaggtggaagctgccgtcatggcagtgcctcgtactgcagaccaggacactagcacagatttggaacaggtgcaccagttcaccaacaccgagacggccaccctcatagagtcctgcaccaacacttgtctaagcactttggacaagcagaccagcacccagactgtggagacgcggacagtagctgtaggagaaggccgtgtcaaggacatcaactcctccaccaagacgcggtccattggtgttggaacgttgctttctggccattctgggtttgacaggccatcagctgtgaagaccaaagagtcaggtgtggggcagataaatattaacgacaactatctggttggtctcaaaatgaggactatagcttgtgggccaccacagttgactgtggggctgacagccagcagaaggagcgtgggggttggggatgaccctgtaggggaatctctggagaacccccagcctcaagctccacttggaatgatgactggcctggatcactacattgagcgtatccagaagctgctggcagaacagcagacactgctggctgagaactacagtgaactggcagaagctttcggggaacctcactcacagatgggctccctcaactctcagctcatcagcaccctgtcgtctatcaactctgtcatgaaatctgcaagcactgaagagctgaggaaccctgacttccagaaaaccagtctgggtaaaatcacaggcaattatttgggatatacctgtaagtgtgggggccttcagtcaggaagtcccttaagctcccagacatcccagcctgagcaagaagtggggacctcagaaggaaagccaatcagcagcctggatgccttccccactcaggaaggtacgctgtctccagtgaacctgacagacgaccagatcgccgctggcctctatgcatgtacaaacaatgaaagtacactgaagtccatcatgaagaagaaagatggtaacaaagattcaaatggcgcaaaaaagaatcttcagtttgttggcattaatggagggtatgaaacaacttcaagtgatgattccagctcagatgaaagctcttcttccgagtcagatgacgagtgtgatgtcattgagtatcctcttgaagaagaggaggaggaggaggatgaagacactcggggaatggcagaagggcaccatgcagttaatattgaaggtttgaagtctgccagggtggaagatgaaatgcaggttcaagaatgtgaacctgagaaggtggaaatcagagagaggtatgaattaagtgaaaagatgttgtctgcatgcaacttactgaaaaatactataaatgaccccaaagctttgaccagcaaagatatgaggttctgtctgaacaccctccagcacgagtggttccgcgtgtccagtcagaagtcagccattccagccatggtgggggactacatagctgcttttgaggccatttccccagatgtcctccgctatgtcatcaacttggcagacggcaacggcaacacagccctccattacagcgtgtcccactccaacttcgagattgtgaagctgctgttagatgccgatgtgtgtaatgtggatcaccagaacaaggcaggctacacccccatcatgttggcggccctcgccgctgtggaagcagagaaggacatgcggattgtggaagaactcttcggctgtggggatgtgaatgccaaagctagtcaggcgggacagacggccctcatgctggcggtcagtcacggacggatagacatggtgaagggccttctggcctgtggggctgatgtcaacatccaggatgacgagggctccacggccctcatgtgtgccagcgagcacgggcacgtggagattgtcaagctgctgctggcccagcccggctgcaacggtcacctagaggacaacgatggcagcactgcgctctcaatcgccctggaagcaggacacaaggacatcgctgttcttctgtatgcccatgtcaactttgcaaaagcccagtctccgggcacccctaggcttggaaggaagacgtctcctggccccacccaccgaggttcatttgattgattgtatgcaaatagccctttatttacatgccactattaagctgctaattgttcctgttggggtgacagatactgaatgtatacgtattgtgcctgagctcaccagcaaacagaagcatcaagcccaggggtaaaggctgaagctttcacagtgcagagactgctagcctgggcacacacacctcctttctggccgtcttctgtgtagggcacactttaacccagtctctgttgctgttgagtctctgctccgttttgtacagtcacagggaattctgatctgaaggggcaccttctgttcactcccacaaagtggtgtctggttctcactgagacgttttaagatttttccacaaatatttatatgtactaaatgtggaaccattagaaagttcttccaaaatctcattccagcatagttttggatttttcttttgtcttattttaaaataaggaagtcgagatgactttgatcattggtaacttgggcctgggccagacaaagtataaaacttacaaaagaatattctcatttggtcttaactaggtagatgtaatatatgactttttataaaaagggtatctatatgaacttgacacagtattttcagcttttgtattccatactaaagccatgaagaactacacgtaacatcatcatttgtattaattgcacaactccaatgctaaaggttggattgtgttagaggaatcggctctgtatttgcctctagagaaacacagtgttctctttgtatttatggattcctttttaccgtgtcacatttactttggtcctctatgtatttaaatgtttgaagtgccttagactcttgccatattttcaaaataaaattccattaagctctttt

SEQ ID NO: 21 - DNA, 2936 bp, Homo sapiens
ggcagtttcccgaccagagagaacgaacgtgtctgcgggcgcgcggggagcagaggcggtggcgggcggcggcggcaccgggagccgccgagtgaccctcccccgcccctctggccccccaccctcccacccgcccgtggcccgcgcccatggccgcgcgcgctccacacaactcaccggagtccgcgccttgcgccgccgaccagttcgcagctccgcgccacggcagccagtctcacctggcggcaccgcccgcccaccgccccggccacagcccctgcgcccacggcagcactcgaggcgaccgcgacagtggtgggggacgctgctgagtggaagagagcgcagcccggccaccggacctacttactcgccttgctgattgtctatttttgcgtttacaacttttctaagaacttttgtatacaaaggaactttttaaaaaagacgcttccaagttatatttaatccaaagaagaaggatctcggccaatttggggttttgggttttggcttcgtttcttctcttcgttgactttggggttcaggtgccccagctgcttcgggctgccgaggaccttctgggcccccacattaatgaggcagccacctggcgagtctgacatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatcccagacagtggatatgacccacactgccagaagagaattcagtattttttacttttcacactgtcttcccgatgagggaaggagcccagccagaaagcactacaatcatggtcaagttcccaactgagtcatcttgtgagtggataatcaggaaaaatgaggaatccaaaagacaaaaatcaaagaacagatggggtctgtgactggatcttctatcattccaattctaaatccgacttgaatattcctggacttacaaaatgccaagggggtgactggaagttgtggatatcagggtataaattatatccgtgagttgggggagggaagaccagaattcccttgaattgtgtattgatgcaatataagcataaaagatcaccttgtattctctttaccttctaaaagccattattatgatgttagaagaagaggaagaaattcaggtacagaaaacatgtttaaatagcctaaatgatggtgcttggtgagtcttggttctaaaggtaccaaacaaggaagccaaagttttcaaactgctgcatactttgacaaggaaaatctatatttgtcttccgatcaacatttatgacctaagtcaggtaatatacctggtttacttctttagcatttttatgcagacagtctgttatgcactgtggtttcagatgtgcaataatttgtacaatggtttattcccaagtatgccttaagcagaacaaatgtgtttttctatatagttccttgccttaataaatatgtaatataaatttaagcaaacgtctattttgtatatttgtaaactacaaagtaaaatgaacattttgtggagtttgtattttgcatactcaaggtgagaattaagttttaaataaacctataatattttatctgaa

SEQ ID NO: 22 - DNA, 2152 bp, Homo sapiens
ggggagctacgtagggcagagaagtcatggcttctccgtccaaaggcaatgacttgttttcgccctacgaggagggcccagcagtggtggccggaccaggccggggctggggcgccgagggggccgcggaggagcgccgcgtcaaggtctccagcctgcccttcagcgtggaggcgctcatgtccgacaagaagccgcccaaggaggcgtccccgctgccggccgaaagcgcctcggccggggccaccctgcggccactgctgctgtcggggcacggcgctcgggaagcgcacagccccgggccgctggtgaagcccttcgagaccgcctcggtcaagtcggaaaattcagaagatggagcggcgtggatgcaggaacccggccgatattcgccgccgccaagacatacgagccctaccacctgcaccctgaggaaacacaagaccaatcggaagccgcgcacgccctttaccacatcccagctcctcgccctggagcgcaagttccgtcagaaacagtacctctccattgcagagcgtgcagagttctccagctctctgaacctcacagagacccaggtcaaaatctggttccagaaccgaagggccaaggcgaaaagactgcaggaggcagaactggaaaagctgaaaatggctgcaaaacctatgctgccctccagcttcagtctccctttccccatcagctcgcccctgcaggcagcgtccatatatggagcatcctacccgttccatagacctgtgcttcccatcccgcctgtgggactctatgccacgccagtgggatatggcatgtaccacctgtggtaaggaagaccagatcaatagactccatgatggatgcttgtttcaaagggtttcctctccctctccacgaaggcagtaccagccagtactcctgctctgctaaccctgcgtgcaccaccctaagcggctaggctgacagggccacactacatagctgaaatttgttctgtaggcggaggcaccaagccctgttttcttggtgtaatcttccagatgcccccttttcctttcacaaagattggctctgatggtttttatgtataaatatatatatataataaaatataatacattttatacagcagacgtaaaaattcaaattattttaaaaggcaaaatttatatacatatgtgctttttttctatatctcaccttcccaaaagacactgtgtaagtccatttgttgtattttcttaaagagggagacaaattatttgcaaaatgtgctaaagtcaatgatttttacgggattattgacttctgcttatggaaaacaaagaaacagacacagtgcacacagaaaatattagatatggagagattattcaaagtgaaggggacacatcatatttctgcattttacttgcattaaaagaaacctctttatatactacagttgttcctatctctccccccgccccccaccgccccaccacacacatatttttaaagtttttccttttttaagaatatttttgtaagaccaatacctgggatgagaagaatcctgagactgcctggaggtgaggtagaaaattagaaatacttcctaattcttctcaaggctgttggtaactttatttcagataattggagagtaaaatgttaaaacctgttgagaggaattgatggtttctgagaaatactaggtacattcatcctcacagattgcaaaggtgatttgggtgggggtttagtaattttctgcttaaaaaatgagtatcttgtaaccattacctatatgctaaatattcttgaacaattagtagatccagaaagaaaaaaaaaatatgctttctctgtgtgtgtacctgttgtatgtcctaaacttattagaaaattttatatacttttttacatgttggggggcagaaggtaaagccatgttttgacttggtgaaaatggggttgtcaaacagcccattaagctccctggtatttcacctcctgtccatctctcccctccctccggtatacctttatccctttgaaagggtgcttgtacaatttgatatattttattgaagagttatctcttattctgaattaaattaagcatttgttttattgcagtaaagtttgtccaaactcaaaaaaaaaaaaaaaa

SEQ ID NO: 23 - DNA, 1199 bp, Homo sapiens
acctctcaagcagccagcgcctgcctgaatctgttctgccccctccccacccatttcaccaccaccatgacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgctttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtaggggcacgtcgcccgctgagctgagtggccagccagtgccattccactccactcaggttcttcagggccagagcccctgcaccctgtttgggctggtgagctgggagttcaggtgggctgctcacagcctccttcagaggccccaccaatttctcggacacttctcagtgtgtggaagctcatgtgggcccctgagggctcatgcctgggaagtgttgtggtgggggctcccaggaggactggcccagagagccctgagatagcggggatcctgaactggactgaataaaacgtggtctcccactgcgccaa

SEQ ID NO: 24 - DNA, 4515 bp, Homo sapiens
ggagtttattcataacgcgctctccaagtatacgtggcaatgcgttgctgggttattttaatcattctaggcatcgttttcctccttatgcctctatcattcctccctatctacactaacatcccacgctctgaacgcgcgcccattaatacccttctttcctccactctccctgggactcttgatcaaagcgcggccctttccccagccttagcgaggcgccctgcagcctggtacgcgcgtggcgtggcggtgggcgcgcagtgcgttctcggtgtggagggcagctgttccgcctgcgatgatttatactcacaggacaaggatgcggtttgtcaaacagtactgctacggaggagcagcagagaaagggagagggtttgagagggagcaaaagaaaatggtaggcgcgcgtagttaattcatgcggctctcttactctgtttacatcctagagctagagtgctcggctgcccggctgagtctcctccccaccttccccaccctccccaccctccccataagcgcccctcccgggttcccaaagcagagggcgtgggggaaaagaaaaaagatcctctctcgctaatctccgcccaccggccctttataatgcgagggtctggacggctgaggacccccgagctgtgctgctcgcggccgccaccgccgggccccggccgtccctggctcccctcctgcctcgagaagggcagggcttctcagaggcttggcgggaaaaagaacggagggagggatcgcgctgagtataaaagccggttttcggggctttatctaactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcctagtatatagtacctagtattataggtactataaaccctaattttttttatttaagtacattttgctttttaaagttgatttttttctattgtttttagaaaaaataaaataactggcaaatatatcattgagccaaatcttaagttgtgaatgttttgtttcgtttcttccccctcccaaccaccaccatccctgtttgttttcatcaattgccccttcagagggtggtcttaagaaaggcaagagttttcctctgttgaaatgggtctgggggccttaaggtctttaagttcttggaggttctaagatgcttcctggagactatgataacagccagagttgacagttagaaggaatggcagaaggcaggtgagaaggtgagaggtaggcaaaggagatacaagaggtcaaaggtagcagttaagtacacaaagaggcataaggactggggagttgggaggaaggtgaggaagaaactcctgttactttagttaaccagtgccagtcccctgctcactccaaacccaggaattctgcccagttgatggggacacggtgggaaccagcttctgctgccttcacaaccaggcgccagtcctgtccatgggttatctcgcaaaccccagaggatctctgggaggaatgctactattaaccctatttcacaaacaaggaaatagaagagctcaaagaggttatgtaacttatctgtagccacgcagataatacaaagcagcaatctggacccattctgttcaaaacacttaacccttcgctatcatgccttggttcatctgggtctaatgtgctgagatcaagaaggtttaggacctaatggacagactcaagtcataacaatgctaagctctatttgtgtcccaagcactcctaagcattttatccctaactctacatcaaccccatgaaggagatactgttgatttccccatattagaagtagagagggaagctgaggcacacaaagactcatccacatgcccaagattcactgatagggaaaagtggaagcgagatttgaacccaggctgtttactcctaacctgtccaagccacctctcagacgacggtaggaatcagctggctgcttgtgagtacaggagttacagtccagtgggttatgttttttaagtctcaacatctaagcctggtcaggcatcagttcccctttttttgtgatttattttgtttttattttgttgttcattgtttaatttttccttttacaatgagaaggtcaccatcttgactcctaccttagccatttgttgaatcagactcatgacggctcctgggaagaagccagttcagatcataaaataaaacatatttattctttgtcatgggagtcattattttagaaactacaaactctccttgcttccatccttttttacatactcatgacacatgctcatcctgagtccttgaaaaggtatttttgaacatgtgtattaattataagcctctgaaaacctatggcccaaaccagaaatgatgttgattatataggtaaatgaaggatgctattgctgttctaattacctcattgtctcagtctcaaagtaggtcttcagctccctgtactttgggattttaatctaccaccacccataaatcaataaataattactttctttga

SEQ ID NO: 25 - DNA, 4522 bp, Homo sapiens
agagacttgagggaggcgctgcgactgacaagcggctctgcccgggaccttctcgctttcatctagcgctgcactcaatggaggggcgggcaccgcagtgcttaatgctgtcttaactagtgtaggaaaacggctcaacccaccgctgccgaaatgaagtataagaatcttatggcaagggccttatatgacaatgtcccagagtgtgccgaggaactggcctttcgcaagggagacatcctgaccgtcatagagcagaacacagggggactggaaggatggtggctgtgctcattacacggtcggcaaggcattgtcccaggcaaccgggtgaagcttctgattggtcccatgcaggagactgcctccagtcacgagcagcctgcctctggactgatgcagcagacctttggccaacagaagctctatcaagtgccaaacccacaggctgctccccgagacaccatctaccaagtgccaccttcctaccaaaatcagggaatttaccaagtccccactggccacggcacccaagaacaagaggtatatcaggtgccaccatcagtgcagagaagcattgggggaaccagtgggccccacgtgggtaaaaaggtgataacccccgtgaggacaggccatggctacgtatacgagtacccatccagataccaaaaggacgtctatgatatccctccttctcataccactcaaggggtatacgacatccctccctcatcagcaaaaggccctgtgttttcagttccagtgggagagataaaacctcaaggggtgtatgacatcccgcctacaaaaggggtatatgccattccgccctctgcttgccgggatgaagcagggcttagggaaaaagactatgacttcccccctcccatgagacaagctggaaggccggacctcagaccggagggggtttatgacattcctccaacctgcaccaagccagcagggaaggaccttcatgtaaaatacaactgtgacattccaggagctgcagaaccggtggctcgaaggcaccagagcctgtccccgaatcacccacccccgcaactcggacagtcagtgggctctcagaacgacgcatatgatgtcccccgaggcgttcagtttcttgagccaccagcagaaaccagtgagaaagcaaacccccaggaaagggatggtgtttatgatgtccctctgcataacccgccagatgctaaaggctctcgggacttggtggatgggatcaaccgattgtctttctccagtacaggcagcacccggagtaacatgtccacgtcttccacctcctccaaggagtcctcactgtcagcctccccagctcaggacaaaaggctcttcctggatccagacacagctattgagagacttcagcggctccagcaggcccttgagatgggtgtctccagcctaatggcactggtcactaccgactggcggtgttacggatatatggaaagacacatcaatgaaatacgcacagcagtggacaaggtggagctgttcctgaaggagtacctccactttgtcaagggagctgttgcaaatgctgcctgcctcccggaactcatcctccacaacaagatgaagcgggagctgcaacgagttgaagactcccaccagatcctgagtcaaaccagccatgacttaaatgagtgcagctggtccctgaatatcttggccatcaacaagccccagaacaagtgtgacgatctggaccggtttgtgatggtggcaaagacggtgcccgatgacgccaagcagctcaccacaaccatcaacaccaacgcagaggccctcttcagacccggccctggcagcttgcatctgaagaatgggccggagagcatcatgaactcaacggagtacccacacggtggctcccagggacagctgctgcatcctggtgaccacaaggcccaggcccacaacaaggcactgcccccaggcctgagcaaggagcaggcccctgactgtagcagcagtgatggttctgagaggagctggatggatgactacgattacgtccacctacagggtaaggaggagtttgagaggcaacagaaagagctattggaaaaagagaatatcatgaaacagaacaagatgcagctggaacatcatcagctgagccagttccagctgttggaacaagagattacaaagcccgtggagaatgacatctcgaagtggaagccctctcagagcctacccaccacaaacagtggcgtgagtgctcaggatcggcagttgctgtgcttctactatgaccaatgtgagacccatttcatttcccttctcaacgccattgacgcactcttcagttgtgtcagctcagcccagcccccgcgaatcttcgtggcacacagcaagtttgtcatcctcagtgcacacaaactggtgttcattggagacacgctgacacggcaggtgactgcccaggacattcgcaacaaagtcatgaactccagcaaccagctctgcgagcagctcaagaccatagtcatggcaaccaagatggccgccctccattaccccagcaccacggccctgcaggaaatggtgcaccaagtgacagacctttctagaaatgcccagctgttcaagcgctctttgctggagatggcaacgttctgagaagaaaaaaaagaggaaggggactgcgttaacggttactaaggaaaactggaaatactgtctggtttttgtaaatgttatctatttttgtagatattttatataaaaatgaaatattttaacattttatgggtcagtcaactttcagaaattcagggagctggagagggaaatctttttttttccccctgagtggttcttatgtatacatagaagtatctgagacataaactgtacagaaaacttgtccacgtgcttttgtatgcccatgtattcatgtttgtttgtagatgtttgtctgatgcatttcattaaaaaaaaaaccatgaattacgaagcaccttagtaagcaccttctaatgctgcattttttttgttgttgttaaaaacataccagctggttataatattgttctccacgtccttgtgatgattctgagcctggcactcccaaatctgggaagcatagtttatttgcaagtgttcaccttccaaatcatgaggcatagcatgacttattcttgtttggaaaactcttttcaaaactgaccatcttaaacacatgatggccaagtgcccaaaagccctcttgcggagcaaatttcagaatatatatgtggatccaagctctgatagttcaggtgctggagggaagagagacctgtgtgtttagaggccaggaccacagttaggattgggttgtttcaatactgagagacagctacaataaaaggagagcaattgcctccctggggctgttcaatcttctgcatttgtgagtggttcagtcatgaggttttccaaaagatgtttttagagttgtaaaaaccatatttgcagcaaagatttacaaaggcgtatcagactatgattgttcaccaaaataggggaatggtttgatccgccagttgcaagtagaggcctttctgactcttaatattcactttggtgctactacccccattacctgagggaaactggccaggtccttgatcatggaactatagagctaccaggacatatcctgctctctaagggaatttattgctatcttgcaccttctttaaaactcacatatgcagacctgacactcaagagtggctagctacacagagtccatctaatttttgcaacttcctgtggccagtgtgtataaccccttccactatctcacagatagtcacagcgtccattccatagtctgtctcctcacatctgttagtattgacacagcacagacaccacaagccatcaggttcttcatggggcaggtgaaatacttctaccccatgggtaaatgtattcacatattaccaagagaagaagcacattatctatgatcttttggcccagttcttatttagcatttttattccagcctacttggaaacatgtttttatttgcaatatatgcctgactgaattaagcttgcttgttttaaacaaccaaatcattggaacagaaaaggatttaaaaaacaagaatgcatgatctcagagtgattaaaaaaaaatcagtggaaataaatgatcatagaaggtgcttttcaaaacaactgctattataattctcaaagtcctactctgccaaaagaagattaaaagtcatacattacattacaaggaaatgttcatgtgggaagagggttgctgaaaatcaacaacgcttgaagttaaaaagtgtgtctttgtagatttcattgtataatgtgtatttcttaggagatggctgacttgattgatctacgctaagtggagacatttcacatttttaaaaccaaatgttcaatctgtattactctttgccgtcttgtatgtagaggctatttttaaatcattaaatttttagatctctgttttcata

SEQ ID NO: 26 - DNA, 3848 bp, Homo sapiens
agaagcctctgctccaccgcggcgagaggcatgggcacgtggctgccgagggtggccgagctctgggaagaaaagcccgtgtgcctctgcatagcgtcgctacagcgctgactcggtgtggattgattggaaaggtttgagggagtacttgggaagcatggtggcacatgatgagactggaggtctcctacctattaaaaggaccatacgagtcctagatgtcaataaccagtccttcagagaacaagaggagccaagcaataaaagagttcgacctctggctcgtgtcacgtccttggcaaatttaatctctcctgtaagaaatggagctgtcagacgttttggtcaaacaatacagtcatttacccttcgtggtgaccacagatccccagcctctgcccagaagttttctagcaggtcaacagtcccaacacccgccaagagaaggagcagtgcactgtggtcagagatgctggacatcaccatgaaggagtctctcaccaccagggagatcagacggcaggaggcaatatatgaaatgtcccgaggtgaacaggatttaattgaggatctcaaacttgcaagaaaggcctatcatgaccccatgttaaagttgtccatcatgtcagaagaggaactcacacatatatttggtgatctggactcttacatacctctgcatgaagatttgttgacaagaataggagaagcaaccaagcctgatggaacagtggagcagattggtcacattctcgtgagctggttaccgcgcttgaatgcctacagaggttactgtagtaaccagctggcagccaaagctcttcttgatcaaaagaaacaggatccaagagtccaagacttcctccagcgatgtctcgagtctcccttcagtcgaaaactagatctttggagtttcctagatatccctcgaagtcgcctagtcaaataccctttactgttaaaagaaattcttaaacacactccaaaagagcaccctgatgttcagcttctggaggatgctatattgataatacagggagtcctctctgatatcaacttgaagaaaggtgaatccgagtgccagtattacatcgacaagctggagtacctggatgaaaagcagagggaccccagaatcgaagcgagcaaagtgctgctgtgccatggggagctgcggagcaagagtggacataaactttacattttcctgtttcaagacatcttggttctgactcggcccgtcacacggaacgaacggcactcttaccaggtttaccggcagccaatcccagtccaagagctagtcctagaagacctgcaggatggagatgtgagaatgggaggctcctttcgaggagctttcagtaactcagagaaagctaaaaatatctttagaattcgcttccatgacccctctccagcccagtctcacactctgcaagccaatgacgtgttccacaagcagcagtggttcaactgtattcgagcggccattgcccccttccagtcggcaggcagtccacctgagctgcagggcctgccggagctgcacgaagagtgtgaggggaaccacccctctgcgaggaaactcacagcccagaggagggcatccacagtttccagtgttactcaggtagaagttgatgaaaacgcttacagatgtggctctggcatgcagatggcagaggacagcaagagcttaaagacacaccagacacagcccggcatccgaagagcgagggacaaagccctttctggtggcaaacggaaagagactttggtgtagagaaggctctgtgtgttaactgatgggagagactgtttgtttataaatgtgtacagttttgttttctcgtaaggggagcatcatagggttactttataccagttgtaacattttcattgtttttggttgttcttttttctttttttaatggcagctaaagatatacagattactgttaaattgcagtccttttttttttaaagatattttcttgaattatttagaacatggtaagcctggtattttttaatcaaacaaaatatttatgaaatgggttttctcttaattctggattcatcatggctttctaataccaattgtaatatttacaatattcaccaaaacttagaattttgcaaatgctggaattctgccagtgtttctttgctaagccttgcatgcaaaatttgaaattttaacattggcacccaaaacctacatggaatgtatgtctggagtatttcaaactttacattgaaacataatttccttggaaaacaaaccataagcctgaggaggtttttatcaactggaatgctttatattagtttgtttttcactgtacattcctcattttacattcatttaacctgccgattatttaatttttttattgtaaagtagtttttagcatttgcttttatttttttactttgatgccttttcaaattggcatgtctttaaagtatttttcttcctgattaaaaatgtgtgtgtatgtgtgtgtgtgtgtgtatatatatatatatttttaaatcacattaattttaccaagtgaaaccaagccatactgtttttgagccaattaagaaaattgccatttttaaagtgtagcatttcagggtaaagacccatgaaatggcttgatgtattctagactactgaaagaaaaccacttcaaagattttgttgaaagttttagtgttgtctgaaatgcaagagggaaggtgattggtagtgagttaaaagaaaaagagaggaaaagagagtagttttgtcttcaagtaaaatgtctggttgtgccagacatttcacaagtgtgaaaggagataggagaagctcaacttgagggcgtgtagtaagttgtagaaggctcgaggggacgtggacttatttgccttggtttgcaatacctgcaaataatgagtttgaaaagaaacaatgaaatgtgttaaaaatttgaccatattagataaattttcgtggatttagtcataagatggaaaaagactggtgaatcttttattacaaaatgtttctgttaaaatgggatcatcatctttgaaaggggggaggaggagtaaaagcccgattataatggtgatcaattcaagtcagtgttgactattctgtgaaatatatttggccagtggaaatgataatcagaaaagactgtaaatagatccatccaaatgatttctctgtacaaatgaatgatactattaaaaaaaaaaaaagcacacacataatcaccctgaaggtttcttgcctggtggctatgttgggggcatttggcaaaatatgctggtgtggcaaaggaggtagtggtaagcctcataaaagttagtaaaaattgtagttcaacctttgaaatcattgatgaatcagcgaaaggtaggtaagtcttgtgttaggaaatcttttaagaacatggtgtaatcagacacagcaattgtattgaaaataactgaacacctgttactttctggataggatagtaagggtagtagtggcaaaggatcctgattaatttcagattttaatgtaagtacctcaggttcttgtctttggaaattcacattcataagtgtatattacaccaatgattgtgatctgcaacaccagaaagcaagatttctaatcatttccagaggtgttgtatagaatagttattccagagttaccttgatcagcttctgaagtttaagaagcaagtaatagttcaagtgttgttaatggaattatgttttatttaaaaccgtatcttcttttggttccctcaaaggtaaaaataagacccagaatctgagtaaatctattaaatgaatgttcttcagaaata

SEQ ID NO: 27 - DNA, 1579 bp, Homo sapiens
agcccacagcagtccgtgccgccgtcccgcccgccagcgccccagcgaggaagcagcgcgcagcccgcggcccagcgcacccgcagcagcgcccgcagctcgtccgcgccatgttccaggcggccgagcgcccccaggagtgggccatggagggcccccgcgacgggctgaagaaggagcggctactggacgaccgccacgacagcggcctggactccatgaaagacgaggagtacgagcagatggtcaaggagctgcaggagatccgcctcgagccgcaggaggtgccgcgcggctcggagccctggaagcagcagctcaccgaggacggggactcgttcctgcacttggccatcatccatgaagaaaaggcactgaccatggaagtgatccgccaggtgaagggagacctggccttcctcaacttccagaacaacctgcagcagactccactccacttggctgtgatcaccaaccagccagaaattgctgaggcacttctgggagctggctgtgatcctgagctccgagactttcgaggaaatacccccctacaccttgcctgtgagcagggctgcctggccagcgtgggagtcctgactcagtcctgcaccaccccgcacctccactccatcctgaaggctaccaactacaatggccacacgtgtctacacttagcctctatccatggctacctgggcatcgtggagcttttggtgtccttgggtgctgatgtcaatgctcaggagccctgtaatggccggactgcccttcacctcgcagtggacctgcaaaatcctgacctggtgtcactcctgttgaagtgtggggctgatgtcaacagagttacctaccagggctattctccctaccagctcacctggggccgcccaagcacccggatacagcagcagctgggccagctgacactagaaaaccttcagatgctgccagagagtgaggatgaggagagctatgacacagagtcagagttcacggagttcacagaggacgagctgccctatgatgactgtgtgtttggaggccagcgtctgacgttatgagcgcaaaggggctgaaagaacatggacttgtatatttgtacaaaaaaaaagttttatttttctaaaaaaagaaaaaagaagaaaaaatttaaagggtgtacttatatccacactgcacactgcctggcccaaaacgtcttattgtggtaggatcagccctcattttgttgcttttgtgaactttttgtaggggacgagaaagatcattgaaattctgagaaaacttcttttaaacctcacctttgtggggtttttggagaaggttatcaaaaatttcatggaaggaccacattttatatttattgtgcttcgagtgactgaccccagtggtatcctgtgacatgtaacagccaggagtgttaagcgttcagtgatgtggggtgaaaagttactacctgtcaaggtttgtgttaccctcctgtaaatggtgtacataatgtattgttggtaattattttggtacttttatgatgtatatttattaaacagatttttacaaatgaaaaaaaaaaaaa

SEQ ID NO: 28 - DNA, 3601 bp, Homo sapiens
aagacttgaacttgaatctcgaaccactgcatctccgactctgcccagactcttcactccgcggcaccctcaaaccccagcccaggccggggcgcacaagccagccagcgcacctgcagtcctcgcccggacgcgccgcgccccctcggaaccaggctctgctccgagcagccttcgcccctcaagccagccacagtccccgccaggccgggtgggcgtcaagatgaaggcggcccgcttcgtgctgcgcagcgctggctcgctcaacggcgccggcctggtgccccgagaggtggagcatttctcgcgctacagcccgtccccgctgtccatgaagcagctactggactttggttcagaaaatgcatgtgaaagaacttcttttgcatttttgcgacaagaattgcctgtgagactcgccaacattctgaaggaaattgatatcctcccgacccaattagtaaatacctcttcagtgcaattggttaaaagctggtatatacagagcctgatggatttggtggaattccatgagaaaagcccagatgaccagaaagcattatcagactttgtagatacactcatcaaagttcgaaatagacaccataatgtagtccctacaatggcacaaggaatcatagagtataaagatgcctgtacagttgacccagtcaccaatcaaaatcttcaatatttcttggatcgattttacatgaaccgtatttctactcggatgctgatgaaccagcacattcttatatttagtgactcacagacaggaaacccaagccacattggaagcattgatcctaactgtgatgtggtagcagtggtccaagatgcctttgagtgttcaaggatgctctgtgatcagtattatttatcatctccagaattaaagcttacacaagtgaatggaaaatttccagaccaaccaattcacatcgtgtatgttccttctcacctccatcatatgctctttgaactatttaagaatgcaatgcgggcaacagttgaacaccaggaaaatcagccttcccttacaccaatagaggttattgttgtcttgggaaaagaagaccttaccattaagatttcagacagaggaggtggtgttcccctgagaattattgaccgcctctttagttatacatactccactgcaccaacgcctgtgatggataattcccggaatgctcctttggctggttttggttacggcttgccaatttctcgtctgtatgcaaagtactttcaaggagatctgaatctctactctttatcaggatatggaacagatgctatcatctacttaaaggctttgtcttctgagtctatagaaaaacttccagtttttaacaagtcagccttcaaacattatcagatgagctctgaggctgatgactggtgtatcccaagcagggaaccaaagaacctggcaaaagaagtggccatgtgaagagggacactcaggacactttacgggatcaaagtgggtctacaccagtgctgcttcctgaatgtttgtgtgtgaacccttgtttcctccaaaacaaacgacagcaacgaaaactccttaatcagaacactgatccaatgaggaatggagcttgtttctgtgacccaggagaacttagtgcaagactacaggagttaacagatggccagctccttattttttaatgtagaataactcctgagtttatatcaaatcctgaagaaataagcctcagttttccatctgtttttgataagaataagaaagggagtgagtgtgaagatggtggttagcagtttcactaagactgatattttaggcctcttgttcacatcaaaagatattggtgtcagaataccagcattttcctgccatgcaaaggattaaaacttagtttacactatgtggttacaaatatatgtcaatgtacattttgaacatatttatgtgctatggaaggaaatgctggtgactaaaataaggtttactctgaaagaggaggaattttattcaaagcattcaaacattttattcaagtgtttcaaaattcaaagcattgtattcaaagttgcagtgaaggcatcaacttatgtaaaaactcagaaggaaggctcctctgataaaaacacagctcctttattatgctgcttttcttgttcactttacacactaagtaaacacttattgtcaggtgcctagtcttgagtgaattgttagatgtgcactgaactcgggatgttggggattggagagagagaattgccaaagtaacagcaaaaatatctcttactttgctttgtttataaataaattagtagattggaaaaactagtgttagggaaagaaatcacatgttcagagcctaattcagtaggaagggcttttctctaccctgaaatgaaggtaatccaaaggcatccattttctaggcttaaaagatatatttttgatatatttaattatattctctacactccagcattaatatgtctgtttaaaaattactaattctcaaatggctcaagaacattagaatttaagtaccttttagagtaattattttaagcaaatagcctggacgtaagagattctcatgccagcatgctttcatttgtcagttgttgtgactgagagataatgaatgacacctgaaatgcatatggtatttttgggagagttaaggtataatttgaaggttggcagaccagttgcgctgattactcttagagaagaagaaatggaaaaatgaaagaaggcaggaaggaaagaaaggatataggaagagagggaagcagaaggcaggcatttttctattttccccacaaattatttcaaaaaaaatctgtattttctgggatatgtcattggcaagaggaagaactggtgttttgaaagcagtatggattctttaaatgcctctcactcttacaagatagtaggctttgagataataaacttacccgtgtcaattaacatttaaactggcatatagaaaaaaaggaggatttttctgcattgtaaaataatcagtatggtttatatgttgaatttgacatttgtgtgtaatttcatggtggcctagtgttgtggtgcttctggtaatggtaatagaagctcaactatttttttgtggatttcagtttttatcatcagaagtcctagacagtgacatttcttaatggtgggagtccagctcatgcatttctgattatacaaaacagtttgcagtaggttatttgtcatttcagttttttactgaaatttgagctaaacatttttacatgtaaatacttgtatttaccaaagatttaaatcagttgattaattaattaactcaaatactgtgaactatctctaaaacactagaaaaaagaaatgttagtatctcaattacaccaactgtgcaaatgaactttgataaaatagaaataatctacattggcctttgtgaaatctggggaagagctttaggattctagtagatggatactgaatactcaggcccacttaaattattaatgtatacattgtgtttttgtctttatgctatgtacagagaaatgtgataattttttataataaatattttttatgatgataaaa

SEQ ID NO: 29 - DNA, 1897 bp, Homo sapiens
gctttgaccgggtcgtggcagccggagtcgtcttcgggacgcgcctgctcttcgcctttcgctgcagtccgtcgatttctttctccaggaagaaaaatggcatccgttgcagttgatccacaaccgagtgtggtgactcgggtggtcaacctgcccttggtgagctccacgtatgacctcatgtcctcagcctatctcagtacaaaggaccagtatccctacctgaagtctgtgtgtgagatggcagagaacggtgtgaagaccatcacctccgtggccatgaccagtgctctgcccatcatccagaagctagagccgcaaattgcagttgccaatacctatgcctgtaaggggctagacaggattgaggagagactgcctattctgaatcagccatcaactcagattgttgccaatgccaaaggcgctgtgactggggcaaaagatgctgtgacgactactgtgactggggccaaggattctgtggccagcacgatcacaggggtgatggacaagaccaaaggggcagtgactggcagtgtggagaagaccaagtctgtggtcagtggcagcattaacacagtcttggggagtcggatgatgcagctcgtgagcagtggcgtagaaaatgcactcaccaaatcagagctgttggtagaacagtacctccctctcactgaggaagaactagaaaaagaagcaaaaaaagttgaaggatttgatctggttcagaagccaagttattatgttagactgggatccctgtctaccaagcttcactcccgtgcctaccagcaggctctcagcagggttaaagaagctaagcaaaaaagccaacagaccatttctcagctccattctactgttcacctgattgaatttgccaggaagaatgtgtatagtgccaatcagaaaattcaggatgctcaggataagctctacctctcatgggtagagtggaaaaggagcattggatatgatgatactgatgagtcccactgtgctgagcacattgagtcacgtactcttgcaattgcccgcaacctgactcagcagctccagaccacgtgccacaccctcctgtccaacatccaaggtgtaccacagaacatccaagatcaagccaagcacatgggggtgatggcaggcgacatctactcagtgttccgcaatgctgcctcctttaaagaagtgtctgacagcctcctcacttctagcaaggggcagctgcagaaaatgaaggaatctttagatgacgtgatggattatcttgttaacaacacgcccctcaactggctggtaggtcccttttatcctcagctgactgagtctcagaatgctcaggaccaaggtgcagagatggacaagagcagccaggagacccagcgatctgagcataaaactcattaaacctgcccctatcactagtgcatgctgtggccagacagatgacaccttttgttatgttgaaattaacttgctaggcaaccctaaattgggaagcaagtagctagtataaaggccctcaattgtagttgtttccagctgaattaagagctttaaagtttctggcattagcagatgatttctgttcacctggtaagaaaagaatgataggcttgtcagagcctatagccagaactcagaaaaaattcaaatgcacttatgttctcattctatggccattgtgttgcctctgttactgtttgtattgaataaaaacatcttcatgtgggctggggtagaaactggtgtctgctctggtgtgatctgaaaaggcgtcttcactgctttatctcatgatgcttgcttgtaaaacttgattttagtttttcatttctcaaataggaatactacctttgaattcaataaaattcactgcaggatagaccagtta

SEQ ID NO: 30 - DNA, 3910 bp, Homo sapiens
ccggctgcgcgcgcagcggtggtggtggcggcgcgatcggccgggctgtaaccgtcgtctgtccgggagcggctggagcggcagcggcggccgggcacggcgcgaggtgacgccacagggcagcggcggcagcggaggcagcggcggcagcaggagacgcagcggcggccgcagcagcagcagcaagacggactcgtggagacgcgccgccgccgccgccgccgggccgggccgggtgtcgcgcgccgaggctgggggggagtcgtcgccgccgccgccaccgctaccgccgccgccgccgccgccgaggtgactgaggagagaggcgcctcctcgctcccgccaccgccggacttcaatgcccagtccccagctcgccagcgtttttcgttggaatatacgttgcacatttatggcgattctgagtgtgagggcagacttctgccaggctcagcacagcattttcgctgacaagtgagcttggaggttctatgtgccataattaacattgccttgaagactcctggacaccgagactggcctcagaaatagttggctttttttttttttaattgcaagcatatttcttttaatgactccagtaaaattaagcatcaagtaaacaagtggaaagtgacctacacttttaacttgtctcactagtgcctaaatgtagtaaaggctgcttaagttttgtatgtagttggattttttggagtccgaaggtatccatctgcagaaattgaggcccaaattgaatttggattcaagtggattctaaatactttgcttatcttgaagagagaagcttcataaggaataaacaagttgaatagagaaaacactgattgataataggcattttagtggtctttttaatgttttctgctgtgaaacatttcaagatttattgatttttttttttcactttccccatcacactcacacgcacgctcacactttttatttgccataatgaaccgtccagcccctgtggagatctcctatgagaacatgcgttttctgataactcacaaccctaccaatgctactctcaacaagttcacagaggaacttaagaagtatggagtgacgactttggttcgagtttgtgatgctacatatgataaagctccagttgaaaaagaaggaatccacgttctagattggccatttgatgatggagctccaccccctaatcagatagtagatgattggttaaacctgttaaaaaccaaatttcgtgaagagccaggttgctgtgttgcagtgcattgtgttgcaggattgggaagggcacctgtgctggttgcacttgctttgattgaatgtggaatgaagtacgaagatgcagttcagtttataagacaaaaaagaaggggagcgttcaattccaaacagctgctttatttggagaaataccgacctaagatgcgattacgcttcagagataccaatgggcattgctgtgttcagtagaaggaaatgtaaacgaaggctgacttgattgtgccatttagagggaactcttggtacctggaaatgtgaatctggaatattacctgtgtcatcaaagtagtgatggattcagtactcctcaaccactctcctaatgattggaacaaaagcaaacaaaaaagaaatctctctataaaatgaataaaatgtttaagaaaagagaaagagaaaaggaattaattcagtgaaggatgattttgctcctagttttggagtttgaatttctgccaggattgaattattttgaaatctcctgtctttttaaactttttcaaaataggtctctaaggaaaaccagcagaacattagcctgtgcaaaaccatctgtttggggagcacactcttccattatgcttggcacatagatctccctgtggtgggattttttttttccctttttttgtgggggagggttggtggtatatttttcccctcttttttccttcctctcctacatctcccttttcccccgatccaagttgtagatggaatagaagcccttgttgctgtagatgtgcgtgcagtctggcagccttaagcccacctgggcacttttagataaaaaaaaaaaaaaaacaaaaaacaacaccaaaaaaacagcagtgatatatatatatatatatatatatatatatatatatatatatatatatatatatatatatatatataatataatatatatatatatatatatattttccaggtggtttttagtctttactgatgaaagggtgttcatgttagtttcttcaaaaccctatctaatactaggcaaagtagccaagagccttttgttttgtttttattttgataaattagtggagaaatggcattttaagaggagtctcttctcaacttacctgagagtcgaattcttctcttccctaaccaatgaagctaagtggttatcccagaaacttgtcttctaaaagggaggactccaggccatcaataaagatgtccaggcagtgagcgtactttttacaccctgtagaattgtgggctgtagcgttactctgattttctgtctagtatcagagaatgctggtagcttaaaatttttattttaggacttgtactctgaattttcaggaaccgtcaaaggagcagcagcaaattcacatattttcgacttgagaaatgcttgtggtatgtgttttccaaactgccccctatatgtaaagttcagtttaaccactgattgccttgttattactaggttttttgagattaaaaaaaaaaaatccctggtttaaaaccaacaatgatgcctagtgagtatgtgtccacaggccataacagggtagaagagagacatcgtgcaacccaatgagtagtgaagggactgtgttgcttgtgaagcggtgtagtagcatttttgcagattcttggctgggtttagtgtactgatctagaaaagctgtttttctgctcctttgtggaaggcagttatgatcaggctgcatggacaaagcaggtagaggggcaccatcaggggctcttgcactattttcacctctaaatattacgtactcagtagtgccctgcttctagggctctgaatacgggcttaaagtcatcttgtcctgctggaatttgctgtgcagagccataagcctcccattttgttagcgtcagctaggccaataggaacagaccgggaccttgtctcacactgatgatacctcacatgttgaccggctatgtgaactgcctatttcctatgctggagttttgatttttaactaaacgcaaatctgtagattctctcctctcccatcccagaaaacaaaacaaaataatgcttttcgaaattgtttctaggactttaaaacataatggtatatccaaaattctttatttcagaatgcaacaatagattccattaatatagactcaagatcaaaacagcatacctgctaagctaagatagatggtgttgattccactgggttttgatcaatacaataacaaacctttttcctttgacatactctgaattttgttgtttggggggagggggtgtgtgtgtgtgtgtgtgtgtgtgtgtgtgtatgtgtgtgtgtgtgtgtgcacgcgcagtgtccatcagtatcagtgcctgcctgagttaggaaaattacattcctggttctgtattgaggagaaggatgtataaagcaacatgaaacattagccctccttttattttaaagactaatgttaattgttcttaaaactggattttttttccttaaagcaatttttttcttttcgatttaatgaagtattgctagctgaagccagtttgacatagagagatgtcagattgatttgaaaggtgtgcagcctgatttaaaaccaaaccctgaacccttttaaagaacaataaaacatattttacacgctc

SEQ ID NO: 31 - DNA, 471 bp, Homo sapiens
acattttctcggccctgccagcccccaggaggaaggtgggtctgaatctagcaccatgacggaactagagacagccatgggcatgatcatagacgtcttttcccgatattcgggcagcgagggcagcacgcagaccctgaccaagggggagctcaaggtgctgatggagaaggagctaccaggcttcctgcagagtggaaaagacaaggatgccgtggataaattgctcaaggacctggacgccaatggagatgcccaggtggacttcagtgagttcatagtgttcgtggctgcaatcacgtctgcctgtcacaagtactttgagaaggcaggactcaaatgatgccctggagatgtcacagattcctggcagagccatggtcccaggcttcccaaaagtgtttgttggcaattattcccctaggctgagcctgctcatgtacctctgattaataaatgcttatgaaatga

SEQ ID NO: 32 - DNA, 5718 bp, Homo sapiens
ggccgagcgcgcggcctggcgcacgatacgccgagccggtctttgagcgctaacgtctttctgtctccccgcggtggtgatgacggtgaaaactgaggctgctaagggcaccctcacttactccaggatgaggggcatggtggcaattctcatcggtgagtgcaggaatcttgcgggacttctgctccaggagacgcaaagtggaaattttttgaaagtcccggatcagattagtgtgtgtggcgccgggacgttatgaagccgtctaaacgtttctttatttctcctccttctatccacagctttcatgaagcagaggaggatgggtctgaacgactttattcagaagattgccaataactcctatgcatgcaaacagtaagttcagaccggattgaggaaataactagtatagtttgaatttgccagcggtaaacattctcatcacggcgtttatcgggaaggcgaagacttcttctggggtggggatctcatttctccttaaattctaatatatttgacacattttaaacattaaagttaatttgctgatttggcttgaactggagatgtaagataaatggttcgtgttggccgaattcacgctttctccatgagcaacaatccttatttctgtatttaatggggtttattattttctttaactgactaatgtattggggtattttcagtttaaacagtgaattatcgggtagaagtcggtagagccagaaactcacttttgatgttggtgtgccccctagtggcgagctggattctaaatcgtgccctttattccctgcagccctgaagttcagtccatcttgaagatctcccaacctcaggagcctgagcttatgaatgccaacccttctcctccagtaagtttttgtatgtgccgtgcatctgtggagaactgtaagggagtcagttagtattcctacattaatggattaaaatagcatttctagaaattagtatcaaggcaggaatgcttcattatgcataacagtgatataaatatttaagtattgagtcagagtattatttttatttttttcctgggcatattttacctcaagtggttattttaaaaggcatatttcataaaaaggttttatctgtctgaaacaacatgactgtgtgcagtttccatactcatttgaaatgtgatgaaatgtagttttgaatgtttatagatgtatggtcatttgcatcagtcatttgtagatgtaacattttctacatcgtttatgttatagatgtcttcctttgaagcaatggtattaaaagaaattctttttttttttttctagccaagtccttctcagcaaatcaaccttggcccgtcgtccaatcctcatgctaaaccatctgactttcacttcttgaaagtgatcggaaagggcagttttggaaaggtaatttcaaatctgaagatcttttggtacacttccttcatgtcctcttttatattctccctggatgaggatcgaaaaatgatttttttaaattgaaatttcaggttcttctagcaagacacaaggcagaagaagtgttctatgcagtcaaagttttacagaagaaagcaatcctgaaaaagaaagaggtgagatgtgcttgatggggctggcattggcggtagacactccttgaataatcttgattctggaatgttggtgccagttgaacatgccactaaatctgaatcgtcattttcctaggagaagcatattatgtcggagcggaatgttctgttgaagaatgtgaagcaccctttcctggtgggccttcacttctctttccagactgctgacaaattgtactttgtcctagactacattaatggtggagaggtgagcaggggggatagaagtcaactcttagtgtctctgcacagcctgctttgttttagtttgagaaaaaagttttcaaagatttttggtggggagaatgttaccagaattagcatttccttcaacctgtcaggttatagttaatagattacttggggccacttcctgcagttgttcttttgctgtgtatgtcaaaactaattaaattacattgcgcaacccagaatgactttgttctgtctcctgcagttgttctaccatctccagagggaacgctgcttcctggaaccacgggctcgtttctatgctgctgaaatagccagtgccttgggctacctgcattcactgaacatcgtttataggtaagcctgagagctcttcaggctaccagttttggtataaaggagacgtagcactggctgtttcatagggccttaaaataatttgtgtttatttgcaacttggttcgctaaaaccagatcccctagcacgtgagctggcttgacttaagtgccaagggggaacagccaagtaggattgtgcctaatccagaatagatgagcagaacaagggctccttttttcttcactacacaactacagtgaacctaaatgcctctaataccttagcaattatctttaagaggatatcttatgaagtgaaattaacttgtgcaactacttttctattcacttttttacagagacttaaaaccagagaatattttgctagattcacagggacacattgtccttactgatttcggactctgcaaggagaacattgaacacaacagcacaacatccaccttctgtggcacgccggaggtaggcgctgtcttggtttggtgcctggtttacccccgccttccaagagagagatgtacaatcatgcacttaactaccaaaaagagtaaactcctctcagagacttcttaatacagttcagtgcaaataaaatacatttgctgtttgatgtagcatgagaaatcccaagtccttctgttcctttactgaaaagtagctgtttgtaagtaagatctgcatcataaaaactttctaatcctaagtaagagatatcaagtgccagcagtttcctaaatgtcagtacacataggtagccagtcaccctcaaaaagtccagcagttttatcaggaaggaatctaaagatatctatcttccaagctggctctgggtctctcagctttttcaaactaaatgtgtggtcgtgggattgcttgctttcgcaggttctaaacgctgtttccctggtctgtttttcagtatctcgcacctgaggtgcttcataagcagccttatgacaggactgtggactggtggtgcctgggagctgtcttgtatgagatgctgtatggcctggtgagtggcacattgggaaccactggaacactgcctgctccctacaatattgccttcacacagcaaaagcagctaagaggcatattggttattttatagttcataagaataatcacttacctggttcttttgtgcatttcacattttactagataggaccacattgaacctgtgtggtggtgaaaaactaccacttattaacatctaccccctaccctccacacacacacacacaaacacacacacgggttgcaaagtagacacttaaatagcaagggaaaagaaagcattgaggtggggagagtttctcaaatcgagcctaatatttattgccgtttatatctttttctctactggtaatgtgtgccatatgaaacttccaattaagtctaaagtaattttccccttctttcagccgcctttttatagccgaaacacagctgaaatgtacgacaacattctgaacaagcctctccagctgaaaccaaatattacaaattccgcaagacacctcctggagggcctcctgcagaaggacaggacaaagcggctcggggccaaggatgacttcgtgagtgatgttttcctgtcctcctgggccggccgggacgtgcactagacctccctgcccttattgaatgcacctgtctaaattaatcttgggtttcttatcaacagatggagattaagagtcatgtcttcttctccttaattaactgggatgatctcattaataagaagattactcccccttttaacccaaatgtggtgagtatctgtctctcttctaagtatagagaagccaagcgatttattttaattcagaattgtctgggggagggttggaaggaatacattggcagatgttttctccataaacctgttattttacctacatagacacatttatcaattcgaagcaccaaaaggcaacaagtgaacattattcttatgtttaactgtgtgtagccttttgagattttgtgcttgaagtgggtgattatggaagttgatataagacttaaacttggtatttaaagcctggtcaagatttccctgtcctgtgtctagtgtgagttcttgacaagagtgtttttcccttcccgtcacagagtgggcccaacgagctacggcactttgaccccgagtttaccgaagagcctgtccccaactccattggcaagtcccctgacagcgtcctcgtcacagccagcgtcaaggaagctgccgaggctttcctaggcttttcctatgcgcctcccacggactctttcctctgaaccctgttagggcttggttttaaaggattttatgtgtgtttccgaatgttttagttagccttttggtggagccgccagctgacaggacatcttacaagagaatttgcacatctctggaagcttagcaatcttattgcacactgttcgctggaattttttgaagagcacattctcctcagtgagctcatgaggttttcatttttattcttccttccaacgtggtgctatctctgaaacgagcgttagagtgccgccttagacggaggcaggagtttcgttagaaagcggacctgttctaaaaaaggtctcctgcagatctgtctgggctgtgatgacgaatattatgaaatgtgccttttctgaagagattgtgttagctccaaagcttttcctatcgcagtgtttcagttctttattttcccttgtggatatgctgtgtgaaccgtcgtgtgagtgtggtatgcctgatcacagatggattttgttataagcatcaatgtgacacttgcaggacactacaacgtgggacattgtttgtttcttccatatttggaagataaatttatgtgtagacttttttgtaagatacggttaataactaaaatttattgaaatggtcttgcaatgactcgtattcagatgcctaaagaaagcattgctgctacaaatatttctatttttagaaagggtttttatggaccaatgccccagttgtcagtcagagccgttggtgtttttcattgtttaaaatgtcacctgtaaaatgggcattatttatgtttttttttttgcattcctgataattgtatgtattgtataaagaacgtctgtacattgggttataacactagtatatttaaacttacaggcttatttgtaatgtaaaccaccattttaatgtactgtaattaacatggttataatacgtacaatccttccctcatcccatcacacaactttttttgtgtgtgataaactgattttggtttgcaataaaaccttgaaaaatatttacatatattgtgtcatgtgttattttgtatattttggttaagggggtaatcatgggttagtttaaaattgaaaaccatgaaaatcctgctgtaatttcctgcttagtggtttgctccaacagcagtggtttctgactccagggagtataggatggcttaagccaccacgtccaggcctttagcagcatt

SEQ ID NO: 33 - DNA, 1700 bp, Homo sapiens
gccgaggctgcctgactggaatgagggtagctgcggcgactgcggcggctggagcggggccggccatggcggtgtggacgcgggccaccaaagcggggctggtggagctgctcctgagggagcgctgggtccgagtggtggccgagctgagcggggagagcctgagcctgacgggcgacgccgccgcggccgagctggagcccgctctgggacccgcggccgccgccttcaacggcctcccaaacggcggcggcgcgggcgactcgctgcccgggagcccaagccgcggcctggggcccccgagcccgccggcgccgcctcggggccccgcgggtgaggcgggcgcgtcgccgcccgtgcgccgggtgcgggtggtgaagcaagaggcgggcggcctgggcatcagcatcaagggcggccgcgagaaccggatgccgatcctcatctccaagatcttccccgggctggctgccgaccagagccgggcgctgcggctgggcgacgccatcctgtcggtgaacggcaccgacctgcgccaggccacccacgaccaggccgtgcaggcgctgaagcgcgcgggcaaggaggtgctgctggaggtcaagttcatccgagaagtaacaccatatatcaagaagccatcattagtatcagatctgccgtgggaaggtgcagccccccagtcaccaagctttagtggcagtgaggactctggttcgccaaaacaccagaacagcaccaaggacaggaagatcatccctctcaaaatgtgctttgctgctagaaacctaagcatgccggatctggaaaacagattgatagagctacattctcctgatagcaggaacacgttgatcctacgctgcaaagatacagccacagcacactcctggttcgtagctatccacaccaacataatggctctcctcccacaggtgttggctgaactcaacgccatgcttggggcaaccagtacagcaggaggcagtaaagaggtgaagcatattgcctggctggcagaacaggcaaaactagatggtggaagacagcaatggagacctgtcctcatggctgtgactgagaaggatttgctgctctatgactgtatgccgtggacaagagatgcctgggcgtcaccatgccacagctacccacttgttgccaccaggttggttcattctggctccggatgtcgatccccctcccttggatctgaccttacatttgctaccaggacaggctctcgacagggcattgagatgcatctcttcagggtggagacacatcgggatctgtcatcctggaccaggatacttgttcagggttgccatgctgctgctgagctgatcaaggaagtctctctaggctgcatgttaaatggccaagaggtgaggcttactattcactatgaaaatgggttcaccatctcaagggaaaatggaggctccagcagcatattgtaccgctacccctttgaaaggctgaagatgtctgctgatgatggcatccgaaatctatacttggattttggtggtcccgagggagaactgaccatggacctgcactcttgtccgaagccgattgtatttgtgttgcacacgtttttatcggccaaagtcactcgtatgggactgcttgtatgagcaacaaaaaatcagaaaagagccttgactgtcacaagaaatatttccacctccaaa

SEQ ID NO: 34 - DNA, 4314 bp, Homo sapiens
agacaggatattcactgctgtggcaaggcctgtagagagtttcgaagttaggaggactcaagacggtccctccctggacttttctgaaggggctcaaaagatgacacgcgccagagctggaaggcgtcgccaattggtccacttttccctcctccctttttgcggatgagaaaactgaggcccaggtttgggatttccagagcccgggatttcccggcaacgcccgacaaccacattcccccggctattctgacccgccccggttccgggacgctccctgggagccgccgccgagggcctgctgggactcccgggggaccccgccgtcggggcagcccccacgcccggcgccgcccgccgggaacggccgccgctgttgcgcacttgcaggggagccggcgactgagggcgaggcagggagggagcaagcggggctgggagggctgctggcgcgggctcgcgcgctgtgtatggtctatcgcaggcagctgacctttgaggaggaaatcgctgctctccgctccttcctgtagtaacagccgccgctgccgccgccgccaggaaccccggccgggagcgagagccgcggggcgcagagccggcccggctgccggacggtgcggccccaccaggtgaacggccatggcgggctggatccaggcccagcagctgcagggagacgcgctgcgccagatgcaggtgctgtacggccagcacttccccatcgaggtccggcactacttggcccagtggattgagagccagccatgggatgccattgacttggacaatccccaggacagagcccaagccacccagctcctggagggcctggtgcaggagctgcagaagaaggcggagcaccaggtgggggaagatgggtttttactgaagatcaagctggggcactacgccacgcagctccagaaaacatatgaccgctgccccctggagctggtccgctgcatccggcacattctgtacaatgaacagaggctggtccgagaagccaacaattgcagctctccggctgggatcctggttgacgccatgtcccagaagcaccttcagatcaaccagacatttgaggagctgcgactggtcacgcaggacacagagaatgagctgaagaaactgcagcagactcaggagtacttcatcatccagtaccaggagagcctgaggatccaagctcagtttgcccagctggcccagctgagcccccaggagcgtctgagccgggagacggccctccagcagaagcaggtgtctctggaggcctggttgcagcgtgaggcacagacactgcagcagtaccgcgtggagctggccgagaagcaccagaagaccctgcagctgctgcggaagcagcagaccatcatcctggatgacgagctgatccagtggaagcggcggcagcagctggccgggaacggcgggccccccgagggcagcctggacgtgctacagtcctggtgtgagaagttggccgagatcatctggcagaaccggcagcagatccgcagggctgagcacctctgccagcagctgcccatccccggcccagtggaggagatgctggccgaggtcaacgccaccatcacggacattatctcagccctggtgaccagcacattcatcattgagaagcagcctcctcaggtcctgaagacccagaccaagtttgcagccaccgtacgcctgctggtgggcgggaagctgaacgtgcacatgaatcccccccaggtgaaggccaccatcatcagtgagcagcaggccaagtctctgcttaaaaatgagaacacccgcaacgagtgcagtggtgagatcctgaacaactgctgcgtgatggagtaccaccaagccacgggcaccctcagtgcccacttcaggaacatgtcactgaagaggatcaagcgtgctgaccggcggggtgcagagtccgtgacagaggagaagttcacagtcctgtttgagtctcagttcagtgttggcagcaatgagcttgtgttccaggtgaagactctgtccctacctgtggttgtcatcgtccacggcagccaggaccacaatgccacggctactgtgctgtgggacaatgcctttgctgagccgggcagggtgccatttgccgtgcctgacaaagtgctgtggccgcagctgtgtgaggcgctcaacatgaaattcaaggccgaagtgcagagcaaccggggcctgaccaaggagaacctcgtgttcctggcgcagaaactgttcaacaacagcagcagccacctggaggactacagtggcctgtccgtgtcctggtcccagttcaacagggagaacttgccgggctggaactacaccttctggcagtggtttgacggggtgatggaggtgttgaagaagcaccacaagccccactggaatgatggggccatcctaggttttgtgaataagcaacaggcccacgacctgctcatcaacaagcccgacgggaccttcttgttgcgctttagtgactcagaaatcgggggcatcaccatcgcctggaagtttgactccccggaacgcaacctgtggaacctgaaaccattcaccacgcgggatttctccatcaggtccctggctgaccggctgggggacctgagctatctcatctatgtgtttcctgaccgccccaaggatgaggtcttctccaagtactacactcctgtgctggctaaagctgttgatggatatgtgaaaccacagatcaagcaagtggtccctgagtttgtgaatgcatctgcagatgctgggggcagcagcgccacgtacatggaccaggccccctccccagctgtgtgcccccaggctccctataacatgtacccacagaaccctgaccatgtactcgatcaggatggagaattcgacctggatgagaccatggatgtggccaggcacgtggaggaactcttacgccgaccaatggacagtcttgactcccgcctctcgccccctgccggtcttttcacctctgccagaggctccctctcatgaatgtttgaatcccacgcttctctttggaaacaatatgcaatgtgaagcggtcgtgttgtgagtttagtaaggctgtgtacactgacacctttgcaggcatgcatgtgcttgtgtgtgtgtgtgtgtgtgtgtccttgtgcatgagctacgcctgcctcccctgtgcagtcctgggatgtggctgcagcagcggtggcctcttttcagatcatggcatccaagagtgcgccgagtctgtctctgtcatggtagagaccgagcctctgtcactgcaggcactcaatgcagccagacctattcctcctgggcccctcatctgctcagcagctatttgaatgagatgattcagaaggggaggggagacaggtaacgtctgtaagctgaagtttcactccggagtgagaagctttgccctcctaagagagagagacagagagacagagagagagaaagagagagtgtgtgggtctatgtaaatgcatctgtcctcatgtgttgatgtaaccgattcatctctcagaagggaggctggggttcattttcgagtagtattttatactttagtgaacgtggactccagactctctgtgaaccctatgagagcgcgtctgggcccggccatgtccttagcacaggggggccgccggtttgagtgagggtttctgagctgctctgaattagtccttgcttggctgcttggccttgggcttcattcaagtctatgatgctgttgcccacgtttcccgggatatatattctctcccctccgttgggccccagccttctttgcttgcctctctgtttgtaaccttgtcgacaaagaggtagaaaagattgggtctaggatatggtgggtggacaggggccccgggacttggagggttggtcctcttgcctcctggaaaaaacaaaaacaaaaaactgcagtgaaagacaagctgcaaatcagccatgtgctgcgtgcctgtggaatctggagtgaggggtaaaagctgatctggtttgactccgctggaggtggggcctggagcaggccttgcgctgttgcgtaactggctgtgttctggtgaggccttgctcccaaccccacacgctcctccctctgaggctgtaggactcgcagtcaggggcagctgaccatggaagattgagagcccaaggtttaaacttctctgaagggaggtggggatgagaagaggggtttttttgtactttgtacaaagaccacacatttgtgtaaacagtgttttggaataaaatatttttttcataaaaaaaaaaaaaaaa

SEQ ID NO: 35 - DNA, 2888 bp, Homo sapiens
atttgtggagaccagccctctggcttggtgagtgaatctggtttacaccggctcctgccctgccttcactcttctcccctgattcaagactcctctgctttggactgaagcactgcaggagtttgtgaccaagaacttcaagagtcaagacagaaggaagccaagggagcagtgcaatggatttctcagtaaaggtagacatagagaaggaggtgacctgccccatctgcctggagctcctgacagaacctctgagcctagattgtggccacagcttctgccaagcctgcatcactgcaaagatcaaggagtcagtgatcatctcaagaggggaaagcagctgtcctgtgtgtcagaccagattccagcctgggaacctccgacctaatcggcatctggccaacatagttgagagagtcaaagaggtcaagatgagcccacaggaggggcagaagagagatgtctgtgagcaccatggaaaaaaactccagatcttctgtaaggaggatggaaaagtcatttgctgggtttgtgaactgtctcaggaacaccaaggtcaccaaacattccgcataaacgaggtggtcaaggaatgtcaggaaaagctgcaggtagccctgcagaggctgataaaggaggatcaagaggctgagaagctggaagatgacatcagacaagagagaaccgcctggaagaattatatccagatcgagagacagaagattctgaaagggttcaatgaaatgagagtcatcttggacaatgaggagcagagagagctgcaaaagctggaggaaggtgaggtgaatgtgctggataacctggcagcagctacagaccagctggtccagcagaggcaggatgccagcacgctcatctcagatctccagcggaggttgaggggatcgtcagtagagatgctgcaggatgtgattgacgtcatgaaaaggagtgaaagctggacattgaagaagccaaaatctgtttccaagaaactaaagagtgtattccgagtaccagatctgagtgggatgctgcaagttcttaaagagctgacagatgtccagtactactgggtggacgtgatgctgaatccaggcagtgccacttcgaatgttgctatttctgtggatcagagacaagtgaaaactgtacgcacctgcacatttaagaattcaaatccatgtgatttttctgcttttggtgtcttcggctgccaatatttctcttcggggaaatattactgggaagtagatgtgtctggaaagattgcctggatcctgggcgtacacagtaaaataagtagtctgaataaaaggaagagctctgggtttgcttttgatccaagtgtaaattattcaaaagtttactccagatatagacctcaatatggctactgggttataggattacagaatacatgtgaatataatgcttttgaggactcctcctcttctgatcccaaggttttgactctctttatggctgtgcctccctgtcgtattggggttttcctagactatgaggcaggcattgtctcatttttcaatgtcacaaaccacggagcactcatctacaagttctctggatgtcgcttttctcgacctgcttatccgtatttcaatccttggaactgcctagtccccatgactgtgtgcccaccgagctcctgagtgttctcattcctttacccacttctgcatagtagcccttgtgctgagactcagattctgcacctgagttcatctctactgagaccatctcttcctttctttccccttcttttacttagaatgtctttgtattcatttgctagggcttccatagcaaagcatcatagattgctgatttaaactgtaattgtattgccgtactgtgggctggaaatcccaaatctagattccagcagagttggttctttctgaggtctgcaaggaagggctctgttccatgcctctctccttggcttgtagaaggcatcttgtccctatgactcttcacattgtctttatgtacatctctgtgcccaagttttccctttttattaagacaccagtcatactggctcagggcccaccgctaatgccttaatgaaatcattttaacattatattctctacaaagaccttatttccaaataagataatatttggaggtattgggaataaaaactccaacatataaatttgaggaaggcacgatttcactcataacaatcttaccctttcttgcaagagatgcttgtacattattttcctaataccttggtttcactagtagtaaacattattattttttttatatttgcaaaggaaacatatctaatccttcctatagaaagaacagtattgctgtaattccttttcttttcttcctcatttcctctgccccttaaaagattgaagaaagagaaacttgtcaactcatatccacgttatctagcaaagtacataagaatctatcactaagtaatgtatccttcagaatgtgttggtttaccagtgacaccccatattcatcacaaaattaaagcaagaagtccatagtaatttatttgctaatagtggatttttaatgctcagagtttctgaggtcaaattttatcttttcacttacaagctctatgatcttaaataatttacttaatgtattttggtgtattttcctcaaattaatattggtgttcaagactatatctaattcctctgatcactttgagaaacaaacttttattaaatgtaaggcacttttctatgaattttaaatataaaaataaatattgttctgattattactgaaaagatgtcagccatttcaatgtcttgggaaacaattttttgtttttgttctgttttctttttgcttcaataaaacaatagctggctcta

SEQ ID NO: 36 - DNA, 2269 bp, Homo sapiens
ctccacaggtccgccccaatccccgctcacacttgggaaacttgggactgcgctggggccgcgtgtggcacctcaggggggcggcccccggcctcaagaggagggggaggagaaggaggaagaggaggaagtgagcccgaaggatccgctcggagctgtttgtccagctgtttctattcgcacccggagcagtacagccagaagggggccgagccgaaggtggctggctttaggcgctaatttccaactcttttcctcacagcttgtcttttccaggcaccctggagtcccctcaggccagctcggtgggcgcgcacctgccagccgcccctgacctcgcaggccaggcgacctccgagcctgagaagatggcccagtccaagctcgattgccgctcacctgtcggcctcgactgctgcaactgctgcctggacctggcccatcggagtgggctccagcgaggcagcagcggggagaacaacaacccgggcagccctacagtgagcaactttcggcagctgcaggaaaagctggtctttgagaacctcaataccgacaagctcaacagcataatgcggcaggattcgctagagccggtgctgcgggacccctgctacctgatcaacgagggcatctgcaaccgcaacatcgaccagaccatgctctccatcctgctcttcttccacagtgcctccggagccagcgtggtggccatagacaacaagatcgaacaggccatggatctggtgaagaatcatctgatgtatgctgtgagagaggaggtggagatcctgaaggagcagatccgagagctggtggagaagaactcccagctagagcgtgagaacaccctgttgaagaccctggcaagcccagagcagctggagaagttccagtcctgtctgagccctgaagagccagctcccgaatccccacaagtgcccgaggcccctggtggttctgcggtgtaagtggctctgtcctcagggtgggcagagccactaaacttgttttacctagttctttccagtttgtttttggctccccaagcatcatctcacgaggagaactttacacctagcacagctggtgccaagagatgtcctaaggacatggccacctgggtccactccagcgacagacccctgacaagagcaggtctctggaggctgagttgcatggggcctagtaacaccaagccagtgagcctctaatgctactgcgccctgggggctcccagggcctgggcaacttagctgcaactggcaaaggagaagggtagtttgaggtgtgacaccagtttgctccagaaagtttaaggggtctgtttctcatctccatggacatcttcaacagcttcacctgacaacgactgttcctatgaagaagccacttgtgttttaagcagaggcaacctctctcttctcctctgtttcgtgaaggcaggggacacagatgggagagattgagccaagtcagccttctgttggttaatatggtataatgcatggctttgtgcacagcccagtgtgggattacagctttgggatgaccgcttacaaagttctgtttggttagtattggcatagtttttctatatagccataaatgcgtatatatacccatagggctagatctgtatcttagtgtagcgatgtatacatatacacatccacctacatgttgaagggcctaaccagccttgggagtattgactggtcccttacctcttatggctaagtctttgactgtgttcatttaccaagttgacccagtttgtcttttaggttaagtaagactcgagagtaaaggcaaggaggggggccagcctctgaatgcggccacggatgccttgctgctgcaaccctttccccagctgtccactgaaacgtgaagtcctgttttgaatgccaaacccaccattcactggtgctgactacatagaatggggttgagagaagatcagtttgggcttcacagtgtcatttgaaaacgttttttgttttgttttgtaattattgtggaaaactttcaagtgaacagaaggatggtgtcctactgtggatgagggatgaacaaggggatggctttgatccaatggagcctgggaggtgtgcccagaaagcttgtctgtagcgggttttgtgagagtgaacactttccactttttgacaccttatcctgatgtatggttccaggatttggattttgattttccaaatgtagcttgaaatttcaataaactttgctctgtttttctaaaaataaa

SEQ ID NO: 37 - DNA, 2572 bp, Homo sapiens
tcccccaaagtgcggatcgggcaataccacagagcctcaggacagggggaagcgccgtcctcttgcccacggacgggtgagctggttcagtggctgagctcaagatgccgttggtgaaaagaaacatcgaccctcggcacttgtgccacacagcactgcccaggggcattaagaacgaactggaatgtgtaaccaacatttccctggcaaatataattagacaactgagtagcctaagtaagtatgctgaagacatatttggagaattattcaatgaagcacacagtttttccttcagagttaactcgttgcaagagcgcgtggaccgattgtctgttagtgtcacacagcttgatcctaaggaggaagaactgtctttgcaagatataacgatgagaaaggctttccggagctccacaattcaagatcagcagcttttcgaccgcaagacgttgcctattccattgcaagaaacatacgatgtgtgtgagcaacctccgcctctcaatatcctcaccccttacagggatgatggtaaggaagggctgaagttttataccaatccttcatatttctttgatctgtggaaagaaaagatgttgcaagatacagaggacaagaggaaggaaaagagaaagcagaagcagaaaaatctagaccgtcctcatgaaccagagaaagtgccaagagcacctcatgacaggcggcgggaatggcagaagctggcccaaggtccagagctggctgaggatgacgctgacctcctacacaagcatattgaagttgccaatggcccagcctctcattatgagacaaggccacagacatacgtggatcatatggacggatcgtactcactctctgccttgccattcagtcagatgagtgaacttctgaccagagctgaggagagggtcttagtcagaccccatgaacctcctccgcctccaccaatgcatggagcaggagatgcaaagcccacacccacctgtatcagttctgctacaggtttgatagagaatcgcccccagtccccagctgcaggcagaacacctgtgtttgtgagccccactcccccacctcctcccccacctcttccgtctgccttgtccacttcttcactaagagcttcaatgacctcaactcctcccccaccagtccctcctccgcctcctcccccagccactgctttgcaggccccagcagtgcccccacctccggctcctcttcagattgcccctggtgttcttcacccagctcctcctccaattgctcctcctctagtgcagccctctcccccagtagctcgagctgccccagtatgcgagactgtgccagttcacccactcccgcaaggtgaagtgcaggggctgcctccacccccaccaccgcctcctctacctccgcctggcatccggccatcatctcctgtcgcagttgcagctcttgctcatcctccctctgggctccatccagcaccatctactgccccgggtccccatgctccattaatgcctccatctcctccatcacaagttctacctgcctctgagccaaagcgccatccatccaccctgcctgtaatcagtgacgcaaggagtgtgctgctggaagccatccgaaaaggtattcagcttcgcaaagtggaagagcagcgtgaacaggaagcaaaacacgagcggattgaaaacgacgtggccaccatcctgtctcgccgcattgctgtggagtatagcgactcggaagatgactcggagttcgatgaagtagactggctggagtaagaaagccacagtgacttggaagatgactcggagttcgatgaagtagactggatggagtaagacagccgccttgctctgtattgcaaggctgaatgcacatgtccctcgtggcgcttgttctttgaaaatgttcggtcattctagtgttttgcttcctattcctcataatgaataacccttttcctccattatttttgatttctatgaaaagtattagcatatatttcaaactaaatattttgtggcttaccttaatttttccccctggaaaaatataatttggtccaataaactactaagtattaagcatggaaagccgttattagagtagcagattcagtttttgatattccttaattgtgcactttgtgatttttttttttttttttaaatttgaagaacgcaactgagactggaatcttgaaggcagctgtgtctgggaacgagccttattccgtttcttgatgttttaaatgaagaaacactgcctcgattatatgaatacactcagaaggaacatgtagcttgtagtgttgaattctcttaaaggaatggttagctttggttatcattttgttgtttttgaatacttgccttatttgaacgtttagcagaaccccttcccacttatatattgtgtggtacagttttgcttgcctatagaagtttaaatttttccatgtgaaatactcagaataagcatatgtgtgacttacataatgtgagaattacaatctgatgtaataaacggttcattttaaaggctaaaaaaaaaaaaaaaaaaaaaa

SEQ ID NO: 38 - DNA, 3660 bp, Homo sapiens
gcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaa

(19) United States
(12) Patent Application Publication
(10) Pub. No.: US 2023/0071390 A1
(43) Pub. Date: Mar. 9, 2023

(54) ASSESSMENT OF PR CELLULAR SIGNALING PATHWAY ACTIVITY USING MATHEMATICAL MODELLING OF TARGET GENE EXPRESSION

(71) Applicant: InnoSIGN B.V., Eindhoven (NL)

(72) Inventors: Márcia Alves De Inda, Eindhoven (NL); Anja Van De Stolpe, Eindhoven (NL); Hendrik Jan Van Ooijen, Eindhoven (NL)

(73) Assignee: InnoSIGN B.V., Eindhoven (NL)

(21) Appl. No.: 17/776,730

(22) PCT Filed: Nov. 12, 2020
(86) PCT No.: PCT/EP2020/081867

§ 371 Date: May 13, 2022

(30) Foreign Application Priority Data
Nov. 14, 2019 (EP) 19209115.5

(51) Int. Cl. C12Q 1/6886; G06N 7/00; G06N 3/00

(52) U.S. Cl. CPC C12Q 1/6886; G06N 7/00; G06N 3/002; C12Q 2600/118; C12Q 2600/158

(57) ABSTRACT

The present invention relates to a computer-implemented method for inferring activity of a PR cellular signaling pathway in a subject based on expression levels of three or more target genes of the PR cellular signaling pathway measured in a sample of the subject. The present invention further relates to an apparatus, to a non-transitory storage medium, and to a computer program for inferring activity of a PR cellular signaling pathway in a subject. The present invention further relates to a kit for measuring expression levels of three or more target genes of the PR cellular signaling pathway in a sample of a subject, to a kit for inferring activity of a PR cellular signaling pathway in a subject, and to the use of such kits in performing the method.

### FIELD OF THE INVENTION

The present invention generally relates to the field of bioinformatics, genomic processing, proteomic processing, and related arts. More particularly, the present invention relates to a computer-implemented method for inferring activity of a PR cellular signaling pathway in a subject performed by a digital processing device, wherein the inferring is based on expression levels of three or more target genes of the PR cellular signaling pathway measured in a sample of the subject. The present invention further relates to an apparatus for inferring activity of a PR cellular signaling pathway in a subject comprising a digital processor configured to perform the method, to a non-transitory storage medium for inferring activity of a PR cellular signaling pathway in a subject storing instructions that are executable by a digital processing device to perform the method, and to a computer program for inferring activity of a PR cellular signaling pathway in a subject comprising program code means for causing a digital processing device to perform the method, when the computer program is run on the digital processing device. The present invention further relates to a kit for measuring expression levels of three or more target genes of the PR cellular signaling pathway in a sample of a subject, to a kit for inferring activity of a PR cellular signaling pathway in a subject, and to uses of the kits in performing the method.

### BACKGROUND OF THE INVENTION

Genomic and proteomic analyses have substantial realized and potential promise for clinical application in medical fields such as oncology, where various cancers are known to be associated with specific combinations of genomic mutations/variations and/or high or low expression levels for specific genes, which play a role in growth and evolution of cancer, e.g., cell proliferation and metastasis.

The progesterone receptor (PR) protein is a member of the nuclear receptor family of transcription factors. The main function of PR is to mediate the effects of progesterone, a progestogen sex hormone which concerns the regulation of events associated with pregnancy and embryogenesis, and it also plays a role in the development of the mammary glands. The PR protein has two main isoforms, PR alpha (PR-A) and PR beta (PR-B), which both originate from the same gene, the progesterone receptor gene (PGR). PR-A is shorter than PR-B, lacking 164 amino acids in the N-terminus (the B-upstream segment or BUS).

PR can modulate gene expression by various mechanisms. The classical mechanism is the canonical PR pathway, where PR is activated by the presence of progestogens in the cell environment. In the absence of a progestogen ligand, PR is in the cytoplasm bound to a complex of chaperone proteins in a conformation favorable for ligand binding. When a progestogen is present, it binds to PR initiating a sequence of transformations that result in PR dimerization, translocation to the nucleus, and initiating transcription of PR target genes by binding to Progesterone Responsive Elements (PRE) in the target gene promoter region. The specific target genes expressed by the canonical PR pathway depend on factors such as the presence of co-ligands (co-activators or co-repressors), PR post-translational modifications (such as SUMOylation and phosphorylation), and steroid receptor interaction (e.g., with estrogen receptor alpha (ERα)) scaffolding (see Dressing, G. E. et al., “Progesterone Receptors Act as Sensors for Mitogenic Protein Kinases in Breast Cancer Models”, Endocrine-Related Cancer, Vol. 16, No. 2, 2009, pages 351-61; Hagan, C. R. and Lange, C. A., “Molecular Determinants of Context-Dependent Progesterone Receptor Action in Breast Cancer”, BMC Medicine, 12:32, 2014). Furthermore, PR can also act through extra-nuclear mechanisms (see Obr, A. E. and Edwards D. P., “The Biology of Progesterone Receptor in the Normal Mammary Gland and in Breast Cancer”, Molecular and Cellular Endocrinology, Vol. 357, No. 1-2, 2012, pages 4-17).

In breast cancer, PR molecular staining is currently used in the clinic to help stratification of breast cancer subtypes. A hormone positive breast cancer, i.e., an estrogen receptor (ER) and PR positive breast cancer, is eligible for anti-estrogen therapy. In this case, PR is merely used as a marker for ER pathway activity. Though there is currently no approved therapy that indicates the use of (anti-)progestogens in the treatment of breast cancer, a number of studies has been carried out to investigate this matter. For example, the anti-progestogen mifepristone has been suggested as a treatment course option for breast cancer patients with high PR-A/PR-B ratio (see Lanari, C. et al., “Antiprogestins in Breast Cancer Treatment: Are We Ready?”, Endocrine-Related Cancer, Vol. 19, No. 3, 2012, pages 35-50; Rojas, P. A. et al., “Progesterone Receptor Isoform Ratio: A Breast Cancer Prognostic and Predictive Factor for Antiprogestin Responsiveness”, Journal of the National Cancer Institute, Vol. 109, No. 7, 2017). Moreover, pre-clinical studies in xenograft models indicate that progesterone has an additive effect over tamoxifen regarding controlling tumor growth (see Mohammed, H. et al., “Progesterone Receptor Modulates ERαAction in Breast Cancer”, Nature, Vol. 523, No. 7560, 2015, pages 313-17). In endometrial cancer, progestogens are commonly used in the treatment of recurrent or advanced endometrial cancer with variable positive response rates (from 26% to 89%) (see Emons, G. et al., “Phase II Study of Fulvestrant 250 Mg/Month in Patients with Recurrent or Metastatic Endometrial Cancer: A Study of the Arbeitsgemeinschaft Gynäkologische Onkologie”, Gynecologic Oncology, Vol. 129, No. 3, 2013, pages 495-99).

WO 2013/162776 A1 discloses a method for determining if a cancer comprises cells expressing an active progesterone receptor and is likely to respond to therapeutic treatment with an anti-progestin, wherein the method is based on the expression level of at least one of KBTBD11, RBPMS2, PLA2G48, FL112684, SH2D4B, RASCD2 and CLDN8 or at least one of THY1, KLF9, SPINK5L.3, PHLDA1 MAPI A, SPRYD5, ATG12, PDK4, MSX2, TUBA3E, TSC22D1, TUBA3D, KHDRBS3, UTS2D, SLC35C1, KIAA0513.

Currently the only method used as an indication of a possibly active PR pathway is PR molecular staining (sometimes PGR expression is also used as a surrogate marker for PR protein expression). Establishment that the PR protein is present in a sample is a necessary condition for an active PR pathway. However, this does not indicate an active PR pathway. Lack of a better marker for PR pathway activity could be a reason for the different rates of response to progestogen treatment in endometrial cancer and for discordant results regarding the role of PR in breast cancer. Indeed, there is evidence that progesterone increases PR pathway activity, but at the same time induces PR ubiquitination that targets the PR protein for degradation (see Patel, B. et al., “Role of Nuclear Progesterone Receptor Isoforms in Uterine Pathophysiology”, Human Reproduction Update, Vol. 21, No. 2, 2015, pages 155-73), which may cause an inverse correlation between the PR protein and PR pathway activation and, thus, hindering the effectiveness of using the PR protein as a surrogate for PR activity. Furthermore, PR activity promoted by means of PR-A, PR-B, or PR-A&B activation could also be a cause for such discordant findings. The development of a PR pathway activation marker, as well as specific PR-A and PR-B activation markers, can be important for attaining a consistent picture of the role played by the PR pathway in e.g. breast cancer, endometrial cancer, and other types of cancers in which PR is involved. More specifically, such makers for PR pathway activation could then be used as companion diagnostics for the indication of (anti-)progestogens in the treatment of cancer and as a prognostic maker.

### SUMMARY OF THE INVENTION

In accordance with a main aspect of the present invention, the above problem is solved by a computer-implemented method for inferring activity of a PR cellular signaling pathway in a subject performed by a digital processing device, wherein the inferring comprises:

receiving expression levels of three or more target genes of the PR cellular signaling pathway measured in a sample of the subject,

determining an activity level of a PR transcription factor (TF) element in the sample of the subject, the PR TF element controlling transcription of the three or more PR target genes, the determining being based on evaluating a calibrated mathematical pathway model relating the expression levels of the three or more PR target genes to the activity level of the PR TF element, and

inferring the activity of the PR cellular signaling pathway in the subject based on the determined activity level of the PR TF element in the sample of the subject,

wherein the calibrated mathematical pathway model is PR-A specific and the three or more, for example, three, four, five, six, seven or more target genes are selected from the group consisting of: BCL2L1, BIRC3, DDIT4, F3, MUC1, NEDD9, SGK1, and TRIM22, preferably, from the group consisting of: BCL2L1, DDIT4, NEDD9, and TRIM22, and/or

wherein the calibrated mathematical pathway model is PR-B specific and the three or more, for example, three, four, five, six, seven, eight, nine, ten, eleven, twelve, thirteen, fourteen, fifteen, sixteen, seventeen, eighteen, nineteen, twenty, twentyone or more target genes are selected from the group consisting of: ARRDC1, ATP1B1, BIRC3, CCND1, CD82, DDIT4, E2F1, F3, FKBP5, GOT1, HSD11B2, KANK1, MSX2, MUC1, MYC, NET1, NFKBIA, PDK4, PLIN2, PTP4A2, SNTB2, and STAT5A, preferably, from the group consisting of: ARRDC1, ATP1B1, CCND1, CD82, E2F1, FKBP5, GOT1, HSD11B2, KANK1, MSX2, MYC, NET1, NFKBIA, PDK4, PLIN2, PTP4A2, SNTB2, and STAT5A, preferably, from the group consisting of: ARRDC1, ATP1B1, CCND1, E2F1, FKBP5, HSD11B2, KANK1, MSX2, MYC, NET1, NFKBIA, PDK4, and PLIN2, preferably, from the group consisting of: CCND1, FKBP5, and MYC, and/or

wherein the calibrated mathematical pathway model is PR-A&B specific and the three or more, for example, three, four, five, six, seven, eight, nine, ten, eleven, twelve, thirteen, fourteen, fifteen, sixteen, seventeen, eighteen, nineteen, twenty, twentyone, twentytwo, twentythree, twentyfour, twentyfive, twentysix, twentyseven, twentyeight, twentynine, thirty, thirtyone, thirtytwo, thirtythree, thirtyfour, thirtyfive, thirtysix, thirtyseven or more target genes are selected from the group consisting of: ABCG2, ACSS1, AK4, ARRDC1, ATP1B1, BCL2L1, BCL6, BIRC3, CCND1, CD82, CDKN1A, DDIT4, E2F1, F3, FKBP5, GOT1, GRB10, HPCAL1, HSD11B2, KANK1, KLF4, MSX2, MUC1, MYC, NEDD9, NET1, NFKBIA, PDK4, PLIN2, PTP4A2, S100P, SGK1, SNTB2, STAT5A, TRIM22, TSC22D3, VASP, and VEGFA, preferably, from the group consisting of: AK4, ARRDC1, ATP1B1, BCL2L1, BCL6, BIRC3, CCND1, CD82, F3, FKBP5, GOT1, GRB10, HSD11B2, KLF4, MUC1, MYC, NEDD9, NET1, PDK4, PTP4A2, S100P, SGK1, SNTB2, STAT5A, TSC22D3, and VASP, or, preferably, from the group consisting of: ABCG2, ACSS1, AK4, ATP1B1, BCL6, CCND1, FKBP5, GRB10, HSD11B2, KANK1, KLF4, MYC, NFKBIA, PDK4, PLIN2, S100P, TSC22D3, and VASP, or, preferably, from the group consisting of: BCL6, CCND1, CDKN1A, FKBP5, MYC, SGK1, and VEGFA, or, preferably, from the group consisting of: BCL6, CCND1, FKBP5, and MYC.

In an embodiment, the invention relates to a computer-implemented method for inferring activity of a PR cellular signaling pathway in a subject performed by a digital processing device, wherein the inferring comprises:

receiving expression levels of three or more target genes of the PR cellular signaling pathway measured in a sample of the subject,

determining an activity level of a PR transcription factor (TF) element in the sample of the subject, the PR TF element controlling transcription of the three or more PR target genes, the determining being based on evaluating a calibrated mathematical pathway model relating the expression levels of the three or more PR target genes to the activity level of the PR TF element, and

inferring the activity of the PR cellular signaling pathway in the subject based on the determined activity level of the PR TF element in the sample of the subject,

wherein the calibrated mathematical pathway model is PR-A specific and the three or more target genes are selected from the group consisting of: BCL2L1, BIRC3, DDIT4, F3, MUC1, NEDD9, SGK1, and TRIM22, and/or

wherein the calibrated mathematical pathway model is PR-B specific and the three or more target genes are selected from the group consisting of: ARRDC1, ATP1B1, CCND1, E2F1, FKBP5, HSD11B2, KANK1, MSX2, MYC, NET1, NFKBIA, PDK4, and PLIN2, and/or

wherein the calibrated mathematical pathway model is PR-A&B specific and the three or more target genes are selected from the group consisting of: ABCG2, ACSS1, AK4, ATP1B1, BCL6, CCND1, FKBP5, GRB10, HSD11B2, KANK1, KLF4, MYC, NFKBIA, PDK4, PLIN2, S100P, TSC22D3, and VASP.

Herein, the “activity level” of a TF element denotes the level of activity of the TF element regarding transcription of its target genes.

The present invention is based on the innovation of the inventors that a suitable way of identifying effects occurring in the PR cellular signaling pathway can be based on a measurement of the signaling output of the PR cellular signaling pathway, which is—amongst others—the transcription of the target genes, which is controlled by a PR transcription factor (TF) element that is controlled by the PR cellular signaling pathway. This innovation by the inventors assumes that the TF activity level is at a quasi-steady state in the sample, which can be detected by means of—amongst others—the expression values of the PR target genes. The PR cellular signaling pathway targeted herein is known to control many functions in many cell types in humans, such as proliferation, differentiation and wound healing. Regarding pathological disorders, such as cancer (e.g., breast, endometrial, ovarian, lung or acute lymphoblastic leukemia (ALL) cancer), the abnormal PR cellular signaling activity plays an important role, which is detectable in the expression profiles of the target genes and thus exploited by means of a calibrated mathematical pathway model.

The present invention makes it possible to determine the activity of the PR cellular signaling pathway in a subject by (i) determining an activity level of a PR TF element in the sample of the subject, wherein the determining is based on evaluating a calibrated mathematical model relating the expression levels of three or more target genes of the PR cellular signaling pathway, the transcription of which is controlled by the PR TF element, to the activity level of the PR TF element, and by (ii) inferring the activity of the PR cellular signaling pathway in the subject based on the determined activity level of the PR TF element in the sample of the subject. This preferably allows improving the possibilities of characterizing patients that have a disease, such as cancer, e.g., breast, endometrial, ovarian, lung or acute lymphoblastic leukemia (ALL) cancer, which is at least partially driven by an abnormal activity of the PR cellular signaling pathway, and that are therefore likely to respond to inhibitors of the PR cellular signaling pathway. Likewise, in cases where the PR cellular signaling pathway is acting as a protective (tumor suppressive) pathway, an abormally low activity of the pathway may preferably be identified using the methods of the present invention and a treatment that increases the activity of the PR cellular signaling pathway, for instance, by providing progesterone to the patient, may be given to the patient. The present invention also indicates that an imbalance of PR-A activity to PR-B activity seems to be an indication of a higher aggressiveness of a cancer. In particular embodiments, treatment determination can be based on a specific PR cellular signaling pathway activity. In a particular embodiment, the PR cellular signaling status can be set at a cutoff value of odds of the PR cellular signaling pathway being active of, for example, 10:1, 5:1, 4:1, 2:1, 1:1, 1:2, 1:4, 1:5, or 1:10.

Herein, the term “Progesterone receptor transcription factor element” or “PR TF element” or “TF element” is defined to be a protein complex containing at least the intracellular PR (PR-A, PR-B), with necessary co-factors, such as p300. Preferably, the term refers to either a protein or protein complex transcriptional factor, activated by binding a specific ligand such as progesterone or by an activating mutation in the PR gene.

The calibrated mathematical pathway model may be a probabilistic model, preferably a Bayesian network model, based on conditional probabilities relating the activity level of the PR TF element and the expression levels of the three or more PR target genes, or the calibrated mathematical pathway model may be based on one or more linear combination(s) of the expression levels of the three or more PR target genes. In particular, the inferring of the activity of the PR cellular signaling pathway may be performed as disclosed in the published international patent application WO 2013/011479 A2 (“Assessment of cellular signaling pathway activity using probabilistic modeling of target gene expression”) or as described in the published international patent application WO 2014/102668 A2 (“Assessment of cellular signaling pathway activity using linear combination(s) of target gene expressions”), the contents of which are herewith incorporated in their entirety. Further details regarding the inferring of cellular signaling pathway activity using mathematical modeling of target gene expression can be found in Verhaegh W. et al., “Selection of personalized patient therapy through the use of knowledge-based computational models that identify tumor-driving signal transduction pathways”, Cancer Research, Vol. 74, No. 11, 2014, pages 2936-2945.

The term “subject”, as used herein, refers to any living being. In some embodiments, the subject is an animal, preferably a mammal. In certain embodiments, the subject is a human being, preferably a medical subject. In still other embodiments, the subject is a cell line.

The term “target gene” as used herein, means a gene whose transcription is directly or indirectly controlled by a PR transcription factor element. The “target gene” may be a “direct target gene” and/or an “indirect target gene” (as described herein). Moreover, the “target genes” may be “direct target genes” and/or “indirect target genes” (as described herein).

Particularly suitable PR target genes are described in the following text passages as well as the examples below (see, e.g., Tables 6 to 8 below).

Thus, according to a preferred embodiment the PR target genes are selected from the group consisting of the PR target genes listed in Table 6, Table 7, or Table 8 below.

Other suitable PR target genes are listed in Table 9, Table 10, Table 11, Table 12, Table 13, Table 14, Table 15, or Table 16 below.

Another aspect of the present invention relates to a method (as described herein), further comprising:

determining whether the PR cellular signaling pathway is operating abnormally in the subject based on the inferred activity of the PR cellular signaling pathway in the subject.

Another aspect of the present invention relates to a method (as described herein), further comprising:

determining a prognostic cancer marker based on a combination of inferred activities of the PR cellular signaling pathway in the subject using two or more of the PR-A specific calibrated mathematical pathway model, the PR-B specific calibrated mathematical pathway model and the PR-A&B specific calibrated mathematical pathway model.

The present invention also relates to a method (as described herein), wherein the combination is a ratio between the inferred activity of the PR cellular signaling pathway in the subject using the PR-A&B specific calibrated mathematical pathway model and the inferred activity of the PR cellular signaling pathway in the subject using the PR-B specific calibrated mathematical pathway model.

The present invention also relates to a method (as described herein), further comprising:

recommending prescribing a drug for the subject that corrects for the abnormal operation of the PR cellular signaling pathway,

wherein the recommending is performed if the PR cellular signaling pathway is determined to be operating abnormally in the subject based on the inferred activity of the PR cellular signaling pathway.

The phrase “the cellular signaling pathway is operating abnormally” refers to the case where the “activity” of the pathway is not as expected, wherein the term “activity” may refer to the activity of the transcription factor complex in driving the target genes to expression, i.e., the speed by which the target genes are transcribed. “Normal” may be when it is inactive in tissue where it is expected to be inactive and active where it is expected to be active. Furthermore, there may be a certain level of activity that is considered “normal”, and anything higher or lower maybe considered “abnormal”.

The present invention also relates to a method (as described herein), wherein the abnormal operation of the PR cellular signaling pathway is an operation in which the PR cellular signaling pathway operates as a tumor promoter in the subject.

The sample(s) to be used in accordance with the present invention can be an extracted sample, that is, a sample that has been extracted from the subject. Examples of the sample include, but are not limited to, a tissue, cells, blood and/or a body fluid of a subject. If the subject is a medical subject that has or may have cancer, it can be, e.g., a sample obtained from a cancer lesion, or from a lesion suspected for cancer, or from a metastatic tumor, or from a body cavity in which fluid is present which is contaminated with cancer cells (e.g., pleural or abdominal cavity or bladder cavity), or from other body fluids containing cancer cells, and so forth, preferably via a biopsy procedure or other sample extraction procedure. The cells of which a sample is extracted may also be tumorous cells from hematologic malignancies (such as leukemia or lymphoma). In some cases, the cell sample may also be circulating tumor cells, that is, tumor cells that have entered the bloodstream and may be extracted using suitable isolation techniques, e.g., apheresis or conventional venous blood withdrawal. Aside from blood, a body fluid of which a sample is extracted may be urine, gastrointestinal contents, or an extravasate. The term “sample”, as used herein, also encompasses the case where e.g. a tissue and/or cells and/or a body fluid of the subject have been taken from the subject and, e.g., have been put on a microscope slide, and where for performing the claimed method a portion of this sample is extracted, e.g., by means of Laser Capture Microdissection (LCM), or by scraping off the cells of interest from the slide, or by fluorescence-activated cell sorting techniques. In addition, the term “sample”, as used herein, also encompasses the case where e.g. a tissue and/or cells and/or a body fluid of the subject have been taken from the subject and have been put on a microscope slide, and the claimed method is performed on the slide. In addition, the term “sample”, as used herein, also encompasses the case where e.g. a cell line and/or cell culture has been generated based on the cells/tissue/body fluid that have been taken from the subject.

In accordance with another disclosed aspect, an apparatus for inferring activity of a PR cellular signaling pathway in a subject comprises a digital processor configured to perform the method of the present invention as described herein.

In accordance with another disclosed aspect, a non-transitory storage medium for inferring activity of a PR cellular signaling pathway in a subject stores instructions that are executable by a digital processing device to perform the method of the present invention as described herein. The non-transitory storage medium may be a computer-readable storage medium, such as a hard drive or other magnetic storage medium, an optical disk or other optical storage medium, a random access memory (RAM), read only memory (ROM), flash memory, or other electronic storage medium, a network server, or so forth. The digital processing device may be a handheld device (e.g., a personal data assistant or smartphone), a notebook computer, a desktop computer, a tablet computer or device, a remote network server, or so forth.

In accordance with another disclosed aspect, a computer program for inferring activity of a PR cellular signaling pathway in a subject comprises program code means for causing a digital processing device to perform the method of the present invention as described herein, when the computer program is run on the digital processing device. The digital processing device may be a handheld device (e.g., a personal data assistant or smartphone), a notebook computer, a desktop computer, a tablet computer or device, a remote network server, or so forth.

In accordance with another disclosed aspect, a kit for measuring expression levels of three or more target genes of the PR cellular signaling pathway in a sample of a subject comprises:

one or more components for determining the expression levels of the three or more PR target genes in the sample of the subject,

wherein the calibrated mathematical pathway model is PR-A specific and the three or more, for example, three, four, five, six, seven or more target genes are selected from the group consisting of: BCL2L1, BIRC3, DDIT4, F3, MUC1, NEDD9, SGK1, and TRIM22, preferably, from the group consisting of: BCL2L1, DDIT4, NEDD9, and TRIM22, and/or wherein the calibrated mathematical pathway model is PR-B specific and the three or more, for example, three, four, five, six, seven, eight, nine, ten, eleven, twelve, thirteen, fourteen, fifteen, sixteen, seventeen, eighteen, nineteen, twenty, twentyone or more target genes are selected from the group consisting of: ARRDC1, ATP1B1, BIRC3, CCND1, CD82, DDIT4, E2F1, F3, FKBP5, GOT1, HSD11B2, KANK1, MSX2, MUC1, MYC, NET1, NFKBIA, PDK4, PLIN2, PTP4A2, SNTB2, and STAT5A, preferably, from the group consisting of: ARRDC1, ATP1B1, CCND1, CD82, E2F1, FKBP5, GOT1, HSD11B2, KANK1, MSX2, MYC, NET1, NFKBIA, PDK4, PLIN2, PTP4A2, SNTB2, and STAT5A, preferably, from the group consisting of: ARRDC1, ATP1B1, CCND1, E2F1, FKBP5, HSD11B2, KANK1, MSX2, MYC, NET1, NFKBIA, PDK4, and PLIN2, preferably, from the group consisting of: CCND1, FKBP5, and MYC, and/or

wherein the calibrated mathematical pathway model is PR-A&B specific and the three or more, for example, three, four, five, six, seven, eight, nine, ten, eleven, twelve, thirteen, fourteen, fifteen, sixteen, seventeen, eighteen, nineteen, twenty, twentyone, twentytwo, twentythree, twentyfour, twentyfive, twentysix, twentyseven, twentyeight, twentynine, thirty, thirtyone, thirtytwo, thirtythree, thirtyfour, thirtyfive, thirtysix, thirtyseven or more target genes are selected from the group consisting of: ABCG2, ACSS1, AK4, ARRDC1, ATP1B1, BCL2L1, BCL6, BIRC3, CCND1, CD82, CDKN1A, DDIT4, E2F1, F3, FKBP5, GOT1, GRB10, HPCAL1, HSD11B2, KANK1, KLF4, MSX2, MUC1, MYC, NEDD9, NET1, NFKBIA, PDK4, PLIN2, PTP4A2, S100P, SGK1, SNTB2, STAT5A, TRIM22, TSC22D3, VASP, and VEGFA, preferably, from the group consisting of: AK4, ARRDC1, ATP1B1, BCL2L1, BCL6, BIRC3, CCND1, CD82, F3, FKBP5, GOT1, GRB10, HSD11B2, KLF4, MUC1, MYC, NEDD9, NET1, PDK4, PTP4A2, S100P, SGK1, SNTB2, STAT5A, TSC22D3, and VASP, or, preferably, from the group consisting of: ABCG2, ACSS1, AK4, ATP1B1, BCL6, CCND1, FKBP5, GRB10, HSD11B2, KANK1, KLF4, MYC, NFKBIA, PDK4, PLIN2, S100P, TSC22D3, and VASP, or, preferably, from the group consisting of: BCL6, CCND1, CDKN1A, FKBP5, MYC, SGK1, and VEGFA, or, preferably, from the group consisting of: BCL6, CCND1, FKBP5, and MYC.

The one or more components or means for measuring the expression levels of the three or more PR target genes can be selected from the group consisting of: a DNA array chip, an oligonucleotide array chip, a protein array chip, an antibody, a plurality of probes, for example, labeled probes, a set of RNA reverse-transcriptase sequencing components, and/or RNA or DNA, including cDNA, amplification primers. In an embodiment, the kit includes a set of labeled probes directed to a portion of an mRNA or cDNA sequence of the three or more PR target genes as described herein. In an embodiment, the kit includes a set of primers and probes directed to a portion of an mRNA or cDNA sequence of the three or more PR target genes. In an embodiment, the labeled probes are contained in a standardized 96-well plate. In an embodiment, the kit further includes primers or probes directed to a set of reference genes. Such reference genes can be, for example, constitutively expressed genes useful in normalizing or standardizing expression levels of the target gene expression levels described herein.

In an embodiment, the kit for measuring the expression levels of three or more target genes of the PR cellular signaling pathway in a sample of a subject comprises:

polymerase chain reaction primers directed to the three or more PR target genes,

probes directed to the three or more PR target genes,

wherein the calibrated mathematical pathway model is PR-A specific and the three or more, for example, three, four, five, six, seven or more target genes are selected from the group consisting of: BCL2L1, BIRC3, DDIT4, F3, MUC1, NEDD9, SGK1, and TRIM22, preferably, from the group consisting of: BCL2L1, DDIT4, NEDD9, and TRIM22, and/or

wherein the calibrated mathematical pathway model is PR-B specific and the three or more, for example, three, four, five, six, seven, eight, nine, ten, eleven, twelve, thirteen, fourteen, fifteen, sixteen, seventeen, eighteen, nineteen, twenty, twentyone or more target genes are selected from the group consisting of: ARRDC1, ATP1B1, BIRC3, CCND1, CD82, DDIT4, E2F1, F3, FKBP5, GOT1, HSD11B2, KANK1, MSX2, MUC1, MYC, NET1, NFKBIA, PDK4, PLIN2, PTP4A2, SNTB2, and STAT5A, preferably, from the group consisting of: ARRDC1, ATP1B1, CCND1, CD82, E2F1, FKBP5, GOT1, HSD11B2, KANK1, MSX2, MYC, NET1, NFKBIA, PDK4, PLIN2, PTP4A2, SNTB2, and STAT5A, preferably, from the group consisting of: ARRDC1, ATP1B1, CCND1, E2F1, FKBP5, HSD11B2, KANK1, MSX2, MYC, NET1, NFKBIA, PDK4, and PLIN2, preferably, from the group consisting of: CCND1, FKBP5, and MYC, and/or

wherein the calibrated mathematical pathway model is PR-A&B specific and the three or more, for example, three, four, five, six, seven, eight, nine, ten, eleven, twelve, thirteen, fourteen, fifteen, sixteen, seventeen, eighteen, nineteen, twenty, twentyone, twentytwo, twentythree, twentyfour, twentyfive, twentysix, twentyseven, twentyeight, twentynine, thirty, thirtyone, thirtytwo, thirtythree, thirtyfour, thirtyfive, thirtysix, thirtyseven or more target genes are selected from the group consisting of: ABCG2, ACSS1, AK4, ARRDC1, ATP1B1, BCL2L1, BCL6, BIRC3, CCND1, CD82, CDKN1A, DDIT4, E2F1, F3, FKBP5, GOT1, GRB10, HPCAL1, HSD11B2, KANK1, KLF4, MSX2, MUC1, MYC, NEDD9, NET1, NFKBIA, PDK4, PLIN2, PTP4A2, S100P, SGK1, SNTB2, STAT5A, TRIM22, TSC22D3, VASP, and VEGFA, preferably, from the group consisting of: AK4, ARRDC1, ATP1B1, BCL2L1, BCL6, BIRC3, CCND1, CD82, F3, FKBP5, GOT1, GRB10, HSD11B2, KLF4, MUC1, MYC, NEDD9, NET1, PDK4, PTP4A2, S100P, SGK1, SNTB2, STAT5A, TSC22D3, and VASP, or, preferably, from the group consisting of: ABCG2, ACSS1, AK4, ATP1B1, BCL6, CCND1, FKBP5, GRB10, HSD11B2, KANK1, KLF4, MYC, NFKBIA, PDK4, PLIN2, S100P, TSC22D3, and VASP, or, preferably, from the group consisting of: BCL6, CCND1, CDKN1A, FKBP5, MYC, SGK1, and VEGFA, or, preferably, from the group consisting of: BCL6, CCND1, FKBP5, and MYC.

In accordance with another disclosed aspect, a kit for inferring activity of a PR cellular signaling pathway in a subject comprises:

the kit of the present invention as described herein, and

the apparatus of the present invention as described herein, the non-transitory storage medium of the present invention as described herein, or the computer program of the present invention as described herein.

In accordance with another disclosed aspect, the kits of the present invention as described herein are used in performing the method of the present invention as described herein.

The present invention as described herein can, e.g., also advantageously be used in at least one of the following activities:

diagnosis based on the inferred activity of the PR cellular signaling pathway in the subject;

prognosis based on the inferred activity of the PR cellular signaling pathway in the subject;

drug prescription based on the inferred activity of the PR cellular signaling pathway in the subject;

prediction of drug efficacy based on the inferred activity of the PR cellular signaling pathway in the subject;

prediction of adverse effects based on the inferred activity of the PR cellular signaling pathway in the subject;

monitoring of drug efficacy;

drug development;

assay development;

pathway research;

cancer staging;

enrollment of the subject in a clinical trial based on the inferred activity of the PR cellular signaling pathway in the subject;

selection of subsequent test to be performed; and

selection of companion diagnostics tests.

Further advantages will be apparent to those of ordinary skill in the art upon reading and understanding the attached figures, the following description and, in particular, upon reading the detailed examples provided herein below.

It shall be understood that the method of claim 1, the apparatus of claim 9, the non-transitory storage medium of claim 10, the computer program of claim 11, the kits of claims 12 to 14, and the use of the kits of claim 15 have similar and/or identical preferred embodiments, in particular, as defined in the dependent claims.

It shall be understood that a preferred embodiment of the present invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

FIG. 1 shows schematically and exemplarily a mathematical model, herein, a Bayesian network model, used to model the transcriptional program of the PR cellular signaling pathway.

FIG. 2 shows a flow chart exemplarily illustrating a process for inferring activity of the PR cellular signaling pathway in a subject based on expression levels of target genes of the PR cellular signaling pathway measured in a sample of a subject.

FIG. 3 shows a flow chart exemplarily illustrating a process for obtaining a calibrated mathematical pathway model as described herein.

FIG. 4 shows a flow chart exemplarily illustrating a process for determining an activity level of a PR transcription factor (TF) element in a sample of a subject as described herein.

FIG. 5 shows a flow chart exemplarily illustrating a process for inferring activity of a PR cellular signaling pathway in a subject using discretized observables.

FIG. 6 shows a flow chart exemplarily illustrating a process for inferring activity of a PR cellular signaling pathway in a subject using continuous observables.

FIG. 7 shows a flow chart exemplarily illustrating a process for determining Cq values from RT-qPCR analysis of the target genes of the PR cellular signaling pathway.

FIG. 8 shows PR cellular signaling pathway activity predictions of trained Bayesian network models on QC pass normal endometrium samples from GSE29921.

FIGS. 9 and 10 show PR cellular signaling pathway activity predictions of trained Bayesian network models on QC pass normal endometrium samples from GSE6364 and GSE51981, respectively.

FIG. 11 shows the progression of the menstrual cycle and the different hormones contributing to it.

FIG. 12 shows PR cellular signaling pathway activity predictions of trained Bayesian network models on fallopian tube samples from GSE10971.

FIG. 13 shows PR cellular signaling pathway activity predictions of trained Bayesian network models on QC pass menopausal normal endometrium samples from GSE12446.

FIG. 14 shows PR cellular sugnaling pathway activity predictions of trained Bayesian network models on QC pass term decidual cells (from normal placentae) samples from GSE65835.

FIG. 15 shows PR cellular signaling pathway activity predictions of trained Bayesian network models on QC pass PR-A and PR-B expressing ishikawa IKPR-AB36 endometrium cell line samples from GSE29435.

FIG. 16 shows PR cellular signaling pathway activity predictions of trained Bayesian network models on QC pass PR-B expressing hMEC breast cell line samples from GSE24468.

FIG. 17 shows PR cellular signaling pathway activity predictions of trained Bayesian network models on QC pass paired normal vs. breast cancer samples from GSE10810.

FIG. 18 shows PR cellular signaling pathway activity predictions of trained Bayesian network models on normal vs. endometrial cancer samples from GSE17025.

FIG. 19 shows PR cellular signaling pathway activity predictions of trained Bayesian network models on paired normal vs. tumor lung cancer samples. Left: Samples from non-smoking female lung cancer in Taiwan from GSE19804; right: Samples from stage I lung adenocarcinoma from GSE27626.

FIG. 20 shows PR cellular signaling pathway activity predictions of trained Bayesian network models on normal and tumor lung cancer samples from GSE30219.

FIG. 21 shows PR cellular signaling pathway activity predictions of trained Bayesian network models on normal vs. idiopathic pulmonary fibrosis (IPF) samples from GSE24206.

FIG. 22 shows PR-B cellular signaling pathway activity predictions of trained Bayesian network models on QC pass paired pre/post letrozole treatment breast cancer samples from GSE10281.

FIG. 23 shows PR cellular signaling pathway activity predictions of trained Bayesian network models as a prognostic risk factor for the progression of lung cancer on QC pass samples from GSE30219.

FIG. 24 shows combined PR cellular signaling pathway activities predictions of trained Bayesian network models as a prognostic risk factor for the progression of lung cancer on QC pass samples from GSE30219.

FIG. 25 shows PR cellular signaling pathway activity predictions of trained Bayesian network models as a prognostic risk factor for the progression of breast cancer on QC pass samples from GSE6532, GSE9195, GSE21653, GSE20685, GSE58812, and EMTAB365.

FIG. 26 shows PR cellular signaling pathway activity predictions of trained Bayesian network models as a prognostic risk factor for t he progression of pediatric acute lymphoblastic leukemia (ALL) on QC pass samples from GSE13576.

FIG. 27 shows PR cellular signaling pathway activity predictions of trained Bayesian network models as a prognostic risk factor in breast cancer on primary normal breast cell cultures samples from GSE13671.

FIG. 28 shows PR cellular signaling pathway activity predictions of linear models as a predictor for progestogen therapy response in endometrial cancer.

FIGS. 29 and 30 show PR cellular signaling pathway activity predictions of trained Bayesian network models calibrated with the alternative calibration set IKAB01M (IPR-AB36 endometrial cell line treated with vehicle control or 1 nM MPA; see Table 4) for the preferred PR-A, PR-B and PR-A&B target gene lists.

FIGS. 31 and 32 show PR cellular pathway activity predictions of trained Bayesian network models calibrated with various alternative target gene lists and the preferred calibration set EmLPxHS (see Table 4).

FIGS. 33 to 38 show PR cellular signaling pathway activity predictions of trained Bayesian network models calibrated with the alternative calibration set IKAB01M on normal endometrium samples from GSE51981 for several randomly selected sets PR-A, PR-B and PR-A&B target genes, each set comprising three randomly selected target genes from the preferred sets (as depicted in Tables 7, 8 and 9).

### DETAILED DESCRIPTION OF EMBODIMENTS

The following examples merely illustrate particularly preferred methods and selected aspects in connection therewith. The teaching provided therein may be used for constructing several tests and/or kits, e.g., to detect, predict and/or diagnose the abnormal activity of the PR cellular signaling pathway. Furthermore, upon using methods as described herein drug prescription can advantageously be guided, drug response prediction and monitoring of drug efficacy (and/or adverse effects) can be made, drug resistance can be predicted and monitored, e.g., to select subsequent test(s) to be performed (like a companion diagnostic test). The following examples are not to be construed as limiting the scope of the present invention.

### 1. Mathematical Model Construction

As described in detail in the published international patent application WO 2013/011479 A2 (“Assessment of cellular signaling pathway activity using probabilistic modeling of target gene expression”), by constructing a probabilistic model, e.g., a Bayesian network model, and incorporating conditional probabilistic relationships between the expression levels of three or more target genes of a cellular signaling pathway, herein, the PR cellular signaling pathway, and the activity level of a transcription factor (TF) element, herein, the PR TF element, the TF element controlling transcription of the three or more target genes of the cellular signaling pathway, such a model may be used to determine the activity of the cellular signaling pathway with a high degree of accuracy. Moreover, the probabilistic model can be readily updated to incorporate additional knowledge obtained by later clinical studies, by adjusting the conditional probabilities and/or adding new nodes to the model to represent additional information sources. In this way, the probabilistic model can be updated as appropriate to embody the most recent medical knowledge.

In another easy to comprehend and interpret approach described in detail in the published international patent application WO 2014/102668 A2 (“Assessment of cellular signaling pathway activity using linear combination(s) of target gene expressions”), the activity of a cellular signaling pathway, herein, the PR cellular signaling pathway, may be determined by constructing and evaluating a linear or (pseudo-)linear model incorporating relationships between expression levels of three or more target genes of the cellular signaling pathway and the level of a transcription factor (TF) element, herein, the PR TF element, the TF element controlling transcription of the three or more target genes of the cellular signaling pathway, the model being based on one or more linear combination(s) of expression levels of the three or more target genes.

In both approaches, the expression levels of the three or more target genes may preferably be measurements of the level of mRNA, which can be the result of, e.g., (RT)-PCR and microarray techniques using probes associated with the target genes mRNA sequences, and of RNA-sequencing. In another embodiment, the expression levels of the three or more target genes can be measured by protein levels, e.g., the concentrations and/or activity of the protein(s) encoded by the target genes.

The aforementioned expression levels may optionally be converted in many ways that might or might not suit the application better. For example, four different transformations of the expression levels, e.g., microarray-based mRNA levels, may be:

“continuous data”, i.e., expression levels as obtained after preprocessing of microarrays using well known algorithms such as MAS5.0 and fRMA,

“z-score”, i.e., continuous expression levels scaled such that the average across all samples is 0 and the standard deviation is 1,

“discrete”, i.e., every expression above a certain threshold is set to 1 and below it to 0 (e.g., the threshold for a probeset may be chosen as the (weighted) median of its value in a set of a number of positive and the same number of negative clinical samples),

“fuzzy”, i.e., the continuous expression levels are converted to values between 0 and 1 using a sigmoid function of the following format: 1/(1+exp((thr−expr)/se)), with expr being the continuous expression levels, thr being the threshold as mentioned before and se being a softening parameter influencing the difference between 0 and 1.

One of the simplest linear models that can be constructed is a model having a node representing the transcription factor (TF) element, herein, the PR TF element, in a first layer and weighted nodes representing direct measurements of the target genes expression levels, e.g., by one probeset that is particularly highly correlated with the particular target gene, e.g., in microarray or (q)PCR experiments, in a second layer. The weights can be based either on calculations from a training data set or based on expert knowledge. This approach of using, in the case where possibly multiple expression levels are measured per target gene (e.g., in the case of microarray experiments, where one target gene can be measured with multiple probesets), only one expression level per target gene is particularly simple. A specific way of selecting the one expression level that is used for a particular target gene is to use the expression level from the probeset that is able to separate active and passive samples of a training data set the best. One method to determine this probeset is to perform a statistical test, e.g., the t-test, and select the probeset with the lowest p-value. The training data set's expression levels of the probeset with the lowest p-value is by definition the probeset with the least likely probability that the expression levels of the (known) active and passive samples overlap. Another selection method is based on odds-ratios. In such a model, one or more expression level(s) are provided for each of the three or more target genes and the one or more linear combination(s) comprise a linear combination including for each of the three or more target genes a weighted term, each weighted term being based on only one expression level of the one or more expression level(s) provided for the respective target gene. If only one expression level is chosen per target gene as described above, the model may be called a “most discriminant probesets” model.

In an alternative to the “most discriminant probesets” model, it is possible, in the case where possibly multiple expression levels are measured per target gene, to make use of all the expression levels that are provided per target gene. In such a model, one or more expression level(s) are provided for each of the three or more target genes and the one or more linear combination(s) comprise a linear combination of all expression levels of the one or more expression level(s) provided for the three or more target genes. In other words, for each of the three or more target genes, each of the one or more expression level(s) provided for the respective target gene may be weighted in the linear combination by its own (individual) weight. This variant may be called an “all probesets” model. It has an advantage of being relatively simple while making use of all the provided expression levels.

Both models as described above have in common that they are what may be regarded as “single-layer” models, in which the activity level of the TF element is calculated based on a linear combination of expression levels of the one or more probeset of the three or more target genes.

After the activity level of the TF element, herein, the PR TF element, has been determined by evaluating the respective model, the determined TF element activity level can be thresholded in order to infer the activity of the cellular signaling pathway, herein, the PR cellular signaling pathway. A preferred method to calculate such an appropriate threshold is by comparing the determined TF element activity levels wlc (weighted linear combination) of training samples known to have a passive cellular signaling pathway and training samples with an active cellular signaling pathway. A method that does so and also takes into account the variance in these groups is given by using a threshold

$$thr = \frac{{σ}_{{wlc}_{pas}} {μ}_{{wlc}_{act}} + {σ}_{{wlc}_{act}} {μ}_{{wlc}_{pas}}}{{σ}_{{wlc}_{pas}} + {σ}_{{wlc}_{act}}} \qquad ( 1 )$$

where α and σ are the standard deviation and the mean of the determined TF element activity levels wlc for the training samples. In case only a small number of samples are available in the active and/or passive training samples, a pseudo count may be added to the calculated variances based on the average of the variances of the two groups:

$$\tilde{v} = \frac{{v}_{{wlc}_{act}} + {v}_{{wlc}_{pas}}}{2} \\ {\tilde{v}}_{{wlc}_{act}} = \frac{x \tilde{v} + ( {n}_{act} - 1 ) {v}_{{wlc}_{act}}}{x + {n}_{act} - 1} \\ {\tilde{v}}_{{wlc}_{pas}} = \frac{x \tilde{v} + ( {n}_{pas} - 1 ) {v}_{{wlc}_{pas}}}{x + {n}_{pas} - 1} \qquad ( 2 )$$

where v is the variance of the determined TF element activity levels wlc of the groups, x is a positive pseudo count, e.g., 1 or 10, and n_{act} and nₚₐₛ are the number of active and passive samples, respectively. The standard deviation a can next be obtained by taking the square root of the variance v.

The threshold can be subtracted from the determined TF element activity levels w/c for ease of interpretation, resulting in a cellular signaling pathway's activity score in which negative values correspond to a passive cellular signaling pathway and positive values correspond to an active cellular signaling pathway.

As an alternative to the above-described “single-layer” models, a “two-layer” may also be used in an example. In such a model, a summary value is calculated for every target gene using a linear combination based on the measured intensities of its associated probesets (“first (bottom) layer”). The calculated summary value is subsequently combined with the summary values of the other target genes of the cellular signaling pathway using a further linear combination (“second (upper) layer”). Again, the weights can be either learned from a training data set or based on expert knowledge or a combination thereof. Phrased differently, in the “two-layer” model, one or more expression level(s) are provided for each of the three or more target genes and the one or more linear combination(s) comprise for each of the three or more target genes a first linear combination of all expression levels of the one or more expression level(s) provided for the respective target gene (“first (bottom) layer”). The model is further based on a further linear combination including for each of the three or more target genes a weighted term, each weighted term being based on the first linear combination for the respective target gene (“second (upper) layer”).

The calculation of the summary values can, in a preferred version of the “two-layer” model, include defining a threshold for each target gene using the training data and subtracting the threshold from the calculated linear combination, yielding the target gene summary. Here the threshold may be chosen such that a negative target gene summary value corresponds to a down-regulated target gene and that a positive target gene summary value corresponds to an up-regulated target gene. Also, it is possible that the target gene summary values are transformed using, e.g., one of the above-described transformations (fuzzy, discrete, etc.), before they are combined in the “second (upper) layer”.

After the activity level of the TF element has been determined by evaluating the “two-layer” model, the determined TF element activity level can be thresholded in order to infer the activity of the cellular signaling pathway, as described above.

In the following, the models described above are collectively denoted as “(pseudo-)linear” models. A more detailed description of the training and use of probabilistic models, e.g., a Bayesian network model, is provided in section 3 below.

### 2. Selection of Target Genes

A transcription factor (TF) is a protein complex (i.e., a combination of proteins bound together in a specific structure) or a protein that is able to regulate transcription from target genes by binding to specific DNA sequences, thereby controlling the transcription of genetic information from DNA to mRNA. The mRNA directly produced due to this action of the TF complex is herein referred to as a “direct target gene” (of the transcription factor). Cellular signaling pathway activation may also result in more secondary gene transcription, referred to as “indirect target genes”. In the following, (pseudo-)linear models or Bayesian network models (as exemplary mathematical models) comprising or consisting of direct target genes as direct links between cellular signaling pathway activity and mRNA level, are preferred, however the distinction between direct and indirect target genes is not always evident. Herein, a method to select direct target genes using a scoring function based on available scientific literature data is presented. Nonetheless, an accidental selection of indirect target genes cannot be ruled out due to limited information as well as biological variations and uncertainties.

Here we propose a list of PR target genes (specifically PR-A specific, PR-B specific, or PR-A&B specific target genes) which are found to be transcribed upon binding of a dimeric protein complex consisting of PR to cellular DNA. The list was generated by manually curating scientific literature found using Pubmed (www.ncbi.nlm.nih.gov/pubmed/) and ScienceDirect (www.sciencedirect.com/). Collected evidence was classified into three categories: 1) PR binds to regulatory region; 2) Presence of a progesterone response element (PRE) in the regulatory region; 3) Gene is differentially regulated by progesterone. When possible, we also annotated which PR isoform was implicated in order to create isoform specific models.

An overall evidence score was computed by adding a normalized literature score (NLscore) and a normalized differential expression score (NDscore) as follows:

1. Normalized Literature score (NLscore): We computed literature scores (Lscores) for each literature evidence category using a weighted sum (see Tables 1 to 3). Per literature source, only the strongest evidence in each category (per gene) was used. The weight given to evidence produced in certain specific settings was corrected as indicated in Tables 1 to 3. We then computed the final Lscore by summing the Lscores obtained for each evidence category plus an extra point for genes with evidence in all three categories (complete evidence), or half a point for genes with evidence in two categories. The normalized literature score (NLscore) was then computed by dividing the Lscore of each gene by the maximum Lscore.

2. Normalized Differential expression score (NDscore): We estimated the differential gene expression score (Dscore) based on the magnitude and significance of the differential expression of the genes in question on a selection of Affymetrix HG1133Plus2 data sets (see Table 4). For each dataset, we calibrated a PR cellular signaling pathway model and used the calibration summary results to estimate differential expression magnitude. Only probesets that were significantly differentially expressed were taken into account. For each calibration set, we computed the proportion of significantly differentially expressed probesets, % sig, the average of odds ratios for the significantly differentially expressed probesets, av(OR), and the average differences in mean expression between PR cellular signaling pathway active and inactive calibration samples (of significantly differentially expressed probesets),

av(diff)=avₚₛ((o̅n̅_{ps,set})−(o̅f̅f̅_{ps,set})), (3)

where (o̅n̅_{ps,set}) is the average expression of active samples for a given probeset ps, on calibration set set and (o̅f̅f̅_{ps,set}) is the average expression of inactive samples for a given probeset ps, on calibration set set.

We then computed a differential expression score for each data set as:

Dscore=% sig*av(diff)*log₂(av(OR))*sign(av(diff)), (4)

and computed an overall score by adding the mean score obtained with the two normal endometrium datasets (GSE6364, GSE11352) to the scores of two other datasets (GSE24468, GSE29435). The normalized score was computed by dividing the absolute value of the Dscore of each gene by the maximum absolute value of the Dscores.

TABLE 1
Weights given depending on evidence strength for category
“PR binds to regulatory region” evidence.

| Evidence type | Strength rank | Weight |
|---|---|---|
| ChIP/PCR | 1 | 1/1 |
| Luciferase assay, CAT assay, EMSA | 2 | 1/2 |
| assay, ChIP/CHIP, ChIP/reChIP, | | |
| ChIP/seq | | |
| H3K4me ChIP | 4 | 1/4 |
| Literature | 8 | 1/8 |
| Extra weight | | |
| PR binds near PR motif | | +half weight |
| Binding is weaker in mutated PR motif | | +half weight |

TABLE 2
Weights given depending on evidence strength for category
“PRE motif in the regulatory region” evidence.

| Evidence type | Strength rank | Weight |
|---|---|---|
| Palindromic/perfect PRE (with sequence) | 1 | 1/1 |
| 1-2 mismatches/non specified PRE (with | 2 | 1/2 |
| sequence) | | |
| putative PRE/Half site PRE (with | 3 | 1/3 |
| sequence) | | |
| Perfect PRE (no sequence) | 3 | 1/3 |
| PRE (no sequence) | 4 | 1/4 |
| Half site PRE | 5 | 1/5 |
| putative PRE | 6 | 1/6 |
| Literature | 8 | 1/8 |
| Extra weight | | |
| PR binds near motif differentially | | +half weight |

TABLE 3
Weights given depending on evidence strength for category
“differential mRNA transcription” evidence.

| Evidence type | Strength rank | Weight |
|---|---|---|
| PCR/Northern blot in CHX | 1 | 1 |
| PCR/Northern blot | 2 | 1/2 |
| Microarray in CHX | 2 | 1/2 |
| Microarray | 3 | 1/3 |
| RNAseq | 3 | 1/3 |
| Western blot/immune fluorescence | 4 | 1/4 |
| RNA PolII ChIP/PCR | 4 | 1/4 |
| RNA PolII ChIP/CHIP | 5 | 1/5 |
| literature | 8 | 1/8 |
| Extra weight | | |
| diff expr. by nuclear PR | | +half weight |
| down regulated by anti-progestogen | | +half weight |

TABLE 4
Affymetrix hg u133 Plus 2.0 calibration sets used in differential expression
analysis for computing Dscore and selecting PR isoform specific models. R5020:
promegestone, synthetic progestin; MPA: medroxyprogesterone acetate,
synthetic progestin; E2: estradiol.

| | | | Active | |
|---|---|---|---|---|
| Name | Datasets | Description | samples | Inactive samples |
| HMB10R | GSE24468 | hMEC normal breast cell line | GSM602697 | GSM602707 |
| | | with a PR-B construct treated | GSM602698 | GSM602708 |
| | | with vehicle control (inactive) | GSM602699 | GSM602709 |
| | | or 10 nM R5020 (active) | GSM602700 | GSM602710 |
| | | | GSM602701 | GSM602711 |
| | | | GSM602702 | GSM602712 |
| | | | GSM602703 | GSM602713 |
| | | | GSM602704 | GSM602714 |
| | | | GSM602705 | GSM602715 |
| | | | GSM602706 | GSM602716 |
| IKAB01M | GSE29435 | IKPR-AB36 endometrial cell | GSM728708 | GSM728705 |
| | | line treated with vehicle control | GSM728709 | GSM728706 |
| | | (inactive) or 1 nM MPA (active) | GSM728710 | GSM728707 |
| EmPxS* | GSE6364 | Normal endometrium during | GSM150221 | GSM150196 |
| | | proliferative (inactive; low | GSM150222 | GSM150197 |
| | | progesterone) or mid secretory | GSM150223 | GSM150198 |
| | | phase (active; high | GSM150224 | GSM150199 |
| | | progesterone) of the menstrual | GSM150225 | GSM150201 |
| | | cycle | | |
| EmExEM* | GSE12446 | Normal endometrium of post- | GSM312568 | GSM312560 |
| | | menopausal women treated with | GSM312667 | GSM312561 |
| | | E2 (inactive) or E2 + MPA | GSM312668 | GSM312563 |
| | | (active) | GSM312669 | GSM312564 |
| | | | GSM312671 | GSM312565 |
| | | | GSM312673 | GSM312566 |
| EmPxMS** | GSE6364 | Healthy endometrium during | GSM150223 | GSM150198 |
| | | proliferative (inactive; low | GSM150224 | GSM150199 |
| | | progesterone) or mid secretory | GSM150225 | GSM150201 |
| | | (active; high progesterone) | GSM150226 | |
| | | phase of the menstrual cycle | GSM150227 | |
| EmLPxHS** | GSE6364 | Healthy endometrium during | GSM150223 | GSM150198 |
| | and | proliferative/low progesterone | GSM150224 | GSM150199 |
| | GSE29981 | phase (active) or mid | GSM150225 | GSM150201 |
| | | secretory/high progesterone | GSM150226 | GSM742055 |
| | | phase (active) of the menstrual | GSM150227 | GSM742057 |
| | | cycle | GSM742061 | GSM742065 |
| | | | GSM742073 | GSM742069 |
| | | | GSM742077 | GSM742079 |

*used only for Dscore;
**used only for isoform specific target list selection

TABLE 5
Selection of PR target genes to determine PR transcription activation, PR
isoform specificity, associated Affymetrix probesets and evidence scores. In bold are genes
with evidence in all categories, in italics are genes with only regulation evidence. The genes
with rank 9, 10, 15 to 17, 19, 25, 28, 29, 34, 36 and 38 are down-regulated genes. The “*”
sign indicates that regulation is in the opposite direction in at least one calibration set.
Isoform specificity is defined as follows: “B > A” means PR-B is a stronger transactivator than
PR-A; “A&B” means PR-A and PR-B have comparable activation strength; “B <> A” means
PR-A regulation is in the opposite direction to PR-B; “B” means PR-B specific; “A” means
PR-A specific.

| | Gene | PR | | Affymetric hg u133 | | | | | Total |
|---|---|---|---|---|---|---|---|---|---|
| rank | Symbol | isoform | Regul. | Plus 2.0 probesets | Lscore | NLscore | Dscore | NDscore | score |
| 1 | FKBP5 | B > A | Up | 204560_at; 224840_at; 224856_at | 15.40 | 1.00 | 45.80 | 1.00 | 2.00 |
| 2 | SGK1 | A > B | Up | 201739_at | 5.46 | 0.35 | 3 1.56 | 0.69 | 1.04 |
| 3 | F3 | B <> A | Up | 204363_at | 7.13 | 1.00 | 16.14 | 1.00 | 0.82 |
| 4 | BIRC3 | B <> A | Up | 210538_s_at; 230499_at | 5.33 | 0.35 | 21.44 | 0.47 | 0.81 |
| 5 | BCL6 | A&B | Up | 203140_at, 215990_s_at | 4.33 | 0.46 | 23.83 | 0.52 | 0.80 |
| 6 | NET1 | B | Up* | 201829_at; 201830_s_at | 4.25 | 0.28 | 20.72 | 0.45 | 0.73 |
| 7 | S100P | A&B | Up | 204351_at | 5.50 | 0.36 | 16.05 | 0.35 | 0.71 |
| 8 | PDK4 | B > A | Up | 225207_at; 205960_at; 1562321_at | 3.50 | 0.23 | 21.45 | 0.47 | 0.70 |
| 9 | MMYC | B > A | Down* | 202431_s_at | 9.83 | 0.64 | −0.93 | −0.02 | 0.66 |
| 10 | CCND1 | B > A | Down | 208711_s_at; 208712_at; 214019_at | 8.12 | 0.53 | −4.58 | −0.10 | 0.63 |
| 11 | HSD11B2 | B > A | Up* | 204130_at | 7.46 | 0.48 | 5.84 | 0.13 | 0.61 |
| 12 | TSC22D3 | A&B | Up | 208763_s_at, 207001_x_at | 4.09 | 0.27 | 13.86 | 0.30 | 0.57 |
| 13 | MUC1 | B <> A | Up | 213693_s_at; 207847_s_at; 211695_x_at | 5.58 | 0.36 | 7.76 | 0.17 | 0.53 |
| 14 | KLF4 | A&B | Up | 220266_s_at, 221841_s_at | 3.67 | 0.24 | 12.40 | 0.27 | 0.51 |
| 15 | MSX2 | B | Down* | 205556_a; 205555_s_at; 210319_x_at | 3.63 | 0.24 | −10.19 | −0.22 | 0.46 |
| 16 | ATP1B1 | B > A | Down | 201242_s_at; 201243_s_at | 4.67 | 0.30 | −7.05 | −0.15 | 0.46 |
| 17 | BCL2L1 | A | Down* | 212312_at; 206665_s_at; 215037_s_at | 6.38 | 0.41 | −1.13 | −0.22 | 0.44 |
| 18 | ABCG2 | A&B | Up* | 209735_at | 3.13 | 0.20 | 10.70 | 0.23 | 0.44 |
| 19 | CDKN1A | A&B | Down* | 1555186_at; 202284_s_at | 5.96 | 0.39 | −1.31 | −0.03 | 0.42 |
| 20 | NFKBIA | B > A | Up* | 201502_s_at | 3.58 | 0.23 | 7.78 | 0.17 | 0.40 |
| 21 | VEGFA | A&B | Up* | 212171_x_at; 210512_s_at; | 5.77 | 0.37 | 0.12 | 0.00 | 0.38 |
| | | | | 211527_x_at; 210513_s_at | | | | | |
| 22 | TRIM22 | A > B | Up* | 213293_s_at | 3.84 | 0.25 | 5.37 | 0.12 | 0.37 |
| 23 | ARRDC1 | B | Up | 226405_s_at | 2.03 | 0.13 | 8.57 | 0.09 | 0.32 |
| 24 | GOT1 | B > A | Up | 208813_at | 2.83 | 0.18 | 6.13 | 0.13 | 0.32 |
| 25 | DDIT4 | B <> A | Down* | 202887_s_at | 2.42 | 0.16 | −7.04 | −0.15 | 0.31 |
| 26 | AK4 | A&B | Up | 204347_at, 204348_s_at, 225342_at, | 1.83 | 0.12 | 8.72 | 0.19 | 0.31 |
| | | | | 230630_at | | | | | |
| 27 | STAT5A | B > A | Up | 201502_s_at | 4.50 | 0.29 | 0.42 | 0.01 | 0.30 |
| 28 | NEDD9 | A | NEDD9 | 202149_at; 202150_s_at; 1569020_at | 2.00 | 0.13 | −5.75 | −0.13 | 0.26 |
| 29 | E2F1 | B | E2F1 | 204947_at; 2028_s_at | 3.42 | 0.22 | −0.60 | −0.01 | 0.23 |
| 30 | KANK1 | B > A | KANK1 | 213005_s_at; 237162_at; 203010_at | 3.00 | 0.19 | 1.29 | 0.03 | 0.22 |
| 31 | SNTB2 | B | SNTB2 | 205315_s_at; 226685_at; 227312_at; | 2.96 | 0.19 | 0.81 | 0.02 | 0.21 |
| | | | | 238925_at | | | | | |
| 32 | GRB10 | A&B | GRB10 | 209409_at, 209410_s_at, | 2.83 | 0.18 | 0.60 | 0.01 | 0.20 |
| | | | | 210999_s_at, 215248_at | | | | | |
| 33 | PLIN2 | B > A | PLIN2 | 209122_at | 1.37 | 0.09 | 4.30 | 0.09 | 0.18 |
| 34 | CD82 | B | CD82 | 203904_x_at | 2.25 | 0.15 | −1.23 | −0.03 | 0.17 |
| 35 | VASP | A&B | VASP | 202205_at | 2.00 | 0.13 | 0.78 | 0.02 | 0.15 |
| 36 | HPCAL1 | A&B | HPCAL1 | 205462_s_at, 212552_at | 1.46 | 0.09 | −1.75 | −0.04 | 0.13 |
| 37 | PTP4A2 | B > A | PTP4A2 | 208615_s_at, 208616_s_at, | 1.17 | 0.08 | 1.44 | 0.03 | 0.11 |
| | | | | 208617_s_at, 1216988_s_at | | | | | |
| 38 | ACSS1 | A&B | ACSS1 | 224882_at; | 1.58 | 0.10 | −0.12 | 0.00 | 0.11 |
| | | | | 234801_s_at | | | | | |

Based on the target gene ranking and PR isoform specificity (see Table 5) we calibrated a series of candidate PR models (using the HMB10R, IKAB01M, EmPxMS, and EmLPxHS calibration sets, see Table 4) based on the following preferred PR-A specific, PR-B specific and PR-A&B target gene lists.

TABLE 6
Preferred “PR-A specific list” of four target
genes of the PR cellular signaling pathway.
Target gene
BCL2L1
DDIT4
NEDD9
TRIM22

TABLE 7
Preferred “PR-B specific list” of thirteen
target genes of the PR cellular signaling pathway.
Target gene
ARRDC1
ATP1B1
CCND1
E2F1
FKBP5
HSD11B2
KANK1
MSX2
MYC
NET1
NFKBIA
PDK4
PLIN2

TABLE 8
Preferred “PR-A&B specific list” of eighteen
target genes of the PR cellular signaling pathway.
Target gene
ABCG2
ACSS1
AK4
ATP1B1
BCL6
CCND1
FKBP5
GRB10
HSD11B2
KANK1
KLF4
MYC
NFKBIA
PDK4
PLIN2
S100P
TSC22D3
VASP

These preferred target gene lists were selected based on their capacity of separating a series of expected active samples from expected inactive samples from the calibration sets of Table 4 according to the following criteria:

(1) best AUC,

(2) best balanced accuracy,

(3) largest difference in activity between expected active and expected inactive samples, and

(4) smallest standard deviation of the average differences between inferred PR cellular signaling pathway activity of active and inactive samples (ground truth) from individual data sets. (The rationale behind this is that average difference in inferred PR cellular signaling pathway activity for active and inactive samples within a data set is preferably similar.)

Other suitable target gene lists include:

TABLE 9
“PR-A specific list” of eight target genes
of the PR cellular signaling pathway
Target gene
BCL2L1
BIRC3
DDIT4
F3
MUC1
NEDD9
SGK1
TRIM22

TABLE 10
“PR-B specific list” of three target genes
of the PR cellular signaling pathway
Target gene
CCND1
FKBP5
MYC

TABLE 11
“PR-B specific list” of eightteen target genes
of the PR cellular signaling pathway.
Target gene
ARRDC1
ATP1B1
CCND1
CD82
E2F1
FKBP5
GOT1
HSD11B2
KANK1
MSX2
MYC
NET1
NFKBIA
PDK4
PLIN2
PTP4A2
SNTB2
STAT5A

TABLE 12
“PR-B specific list” of twentytwo target genes
of the PR cellular signaling pathway.
Target gene
ARRDC1
ATP1B1
BIRC3
CCND1
CD82
DDIT4
E2F1
F3
FKBP5
GOT1
HSD11B2
KANK1
MSX2
MUC1
MYC
NET1
NFKBIA
PDK4
PLIN2
PTP4A2
SNTB2
STAT5A

TABLE 13
“PR-A&B specific list” of four target genes
of the PR cellular signaling pathway.
Target gene
BCL6
CCND1
FKBP5
MYC

TABLE 14
“PR-A&B specific list” of seven target genes
of the PR cellular signaling pathway.
Target gene
BCL6
CCND1
CDKN1A
FKBP5
MYC
SGK1
VEGFA

TABLE 15
“PR-A&B specific list” of twentysix target genes
of the PR cellular signaling pathway.
Target gene
AK4
ARRDC1
ATP1B1
BCL2L1
BCL6
BIRC3
CCND1
CD82
F3
FKBP5
GOT1
GRB10
HSD11B2
KLF4
MUC1
MYC
NEDD9
NET1
PDK4
PTP4A2
S100P
SGK1
SNTB2
STAT5A
TSC22D3
VASP

TABLE 16
“PR-A&B specific list” of thirtyeight target genes
of the PR cellular signaling pathway.
Target gene
ABCG2
ACSS1
AK4
ARRDC1
ATP1B1
BCL2L1
BCL6
BIRC3
CCND1
CD82
CDKN1A
DDIT4
E2F1
F3
FKBP5
GOT1
GRB10
HPCAL1
HSD11B2
KANK1
KLF4
MSX2
MUC1
MYC
NEDD9
NET1
NFKBIA
PDK4
PLIN2
PTP4A2
S100P
SGK1
SNTB2
STAT5A
TRIM22
TSC22D3

Target genes from the preferred gene sets depicted in Tables 7, 8 and 9 were randomly distributed in sets of three target genes as depicted in Tables 17, 18 and 19 below, in order to test whether a selection of three target genes can be used in the calibrated pathway model.

TABLE 17
PR-A randomly selected sets (lists) of three target genes,
selected from the gene list presented in Table 9.

| List | genes | | |
|---|---|---|---|
| Ag03a | BCL2L1 | NEDD9 | SGK1 |
| Ag03b | BCL2L1 | BIRC3 | F3 |
| Ag03c | BCL2L1 | MUC1 | NEDD9 |
| Ag03d | MUC1 | DDIT4 | TRIM22 |
| Ag03e | BCL2L1 | BIRC3 | SGK1 |
| Ag03f | F3 | SGK1 | TRIM22 |
| Ag03g | MUC1 | SGK1 | DDIT4 |
| Ag03h | BCL2L1 | BIRC3 | F3 |

TABLE 18
PR-B randomly selected sets (lists) of three target genes,
selected from the gene list presented in Table 7.

| List | genes | | |
|---|---|---|---|
| Bg03a | FKBP5 | MSX2 | NFKBIA |
| Bg03b | FKBP5 | MSX2 | KANK1 |
| Bg03c | NET1 | PDK4 | MSX2 |
| Bg03d | PDK4 | CCND1 | ARRDC1 |
| Bg03e | CCND1 | HSD11B2 | ATP1B1 |
| Bg03f | FKBP5 | NET1 | PLIN2 |
| Bg03g | PDK4 | KANK1 | PLIN2 |
| Bg03h | NET1 | MSX2 | PLIN2 |

TABLE 19
PR-AB randomly selected sets (lists) of three target genes,
selected from the gene list presented in Table 8.

| List | genes | | |
|---|---|---|---|
| ABg03a | FKBP5 | BCL6 | S100P |
| ABg03b | BCL6 | NFKBIA | ACSS1 |
| ABg03c | PDK4 | NFKBIA | KANK1 |
| ABg03d | ABCG2 | GRB10 | ACSS1 |
| ABg03e | CCND1 | TSC223D | ATP1B1 |
| ABg03f | GRB10 | PLIN2 | VASP |
| ABg03g | PLIN2 | VASP | ACSS1 |
| ABg03h | BCL6 | ATP1B1 | AK4 |

### 3. Training and Using the Mathematical Model

Before the mathematical model can be used to infer the activity of the cellular signaling pathway, herein, the PR cellular signaling pathway, in a subject, the model must be appropriately trained.

If the mathematical pathway model is a probabilistic model, e.g., a Bayesian network model, based on conditional probabilities relating the activity level of the PR TF element and expression levels of three or more target genes of the PR cellular signaling pathway measured in the sample of the subject, the training may preferably be performed as described in detail in the published international patent application WO 2013/011479 A2 (“Assessment of cellular signaling pathway activity using probabilistic modeling of target gene expression”).

If the mathematical pathway model is based on one or more linear combination(s) of expression levels of three or more target genes of the PR cellular signaling pathway measured in the sample of the subject, the training may preferably be performed as described in detail in the published international patent application WO 2014/102668 A2 (“Assessment of cellular signaling pathway activity using linear combination(s) of target gene expressions”).

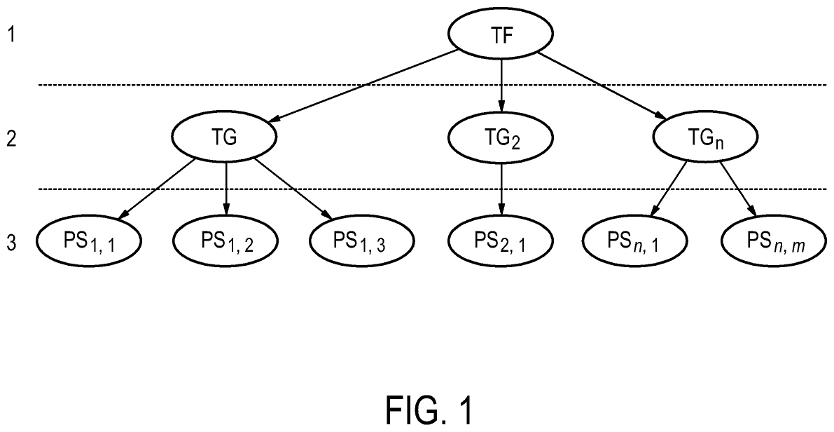

Herein, an exemplary Bayesian network model as shown in FIG. 1 was used to model the transcriptional program of the PR cellular signaling pathway in a simple manner. The model consists of three types of nodes: (a) a transcription factor (TF) element (with states “absent” and “present”) in a first layer 1; (b) target genes TG₁, TG₂, TGₙ (with states “down” and “up”) in a second layer 2, and; (c) measurement nodes linked to the expression levels of the target genes in a third layer 3. These can be microarray probesets PS_{1,1}, PS_{1,2}, PS_{1,3}, PS_{2,1}, PS_{n,1}, PS_{n,m} (with states “low” and “high”), as preferably used herein, but could also be other gene expression measurements such as RNAseq or RT-qPCR.

A suitable implementation of the mathematical model, herein, the exemplary Bayesian network model, is based on microarray data. The model describes (i) how the expression levels of the target genes depend on the activation of the TF element, and (ii) how probeset intensities, in turn, depend on the expression levels of the respective target genes. For the latter, probeset intensities may be taken from fRMA pre-processed Affymetrix HG-U133Plus2.0 microarrays, which are widely available from the Gene Expression Omnibus (GEO, www.ncbi.nlm.nih.gov/geo) and ArrayExpress (www.ebi.ac.uk/arrayexpress).

As the exemplary Bayesian network model is a simplification of the biology of a cellular signaling pathway, herein, the PR cellular signaling pathway, and as biological measurements are typically noisy, a probabilistic approach was opted for, i.e., the relationships between (i) the TF element and the target genes, and (ii) the target genes and their respective probesets, are described in probabilistic terms. Furthermore, it was assumed that the activity of the oncogenic cellular signaling pathway which drives tumor growth is not transiently and dynamically altered, but long term or even irreversibly altered. Therefore the exemplary Bayesian network model was developed for interpretation of a static cellular condition. For this reason complex dynamic cellular signaling pathway features were not incorporated into the model.

Once the exemplary Bayesian network model is built and calibrated (see below), the model can be used on microarray data of a new sample by entering the probeset measurements as observations in the third layer 3, and inferring backwards in the model what the probability must have been for the TF element to be “present”. Here, “present” is considered to be the phenomenon that the TF element is bound to the DNA and is controlling transcription of the cellular signaling pathway's target genes, and “absent” the case that the TF element is not controlling transcription. This probability is hence the primary read-out that may be used to indicate activity of the cellular signaling pathway, herein, the PR cellular signaling pathway, which can next be translated into the odds of the cellular signaling pathway being active by taking the ratio of the probability of it being active vs. it being passive (i.e., the odds are given by p/(1−p), where p is the predicted probability of the cellular signaling pathway being active).

In the exemplary Bayesian network model, the probabilistic relations have been made quantitative to allow for a quantitative probabilistic reasoning. In order to improve the generalization behavior across tissue types, the parameters describing the probabilistic relationships between (i) the TF element and the target genes have been carefully hand-picked. If the TF element is “absent”, it is most likely that the target gene is “down”, hence a probability of 0.95 is chosen for this, and a probability of 0.05 is chosen for the target gene being “up”. The latter (non-zero) probability is to account for the (rare) possibility that the target gene is regulated by other factors or that it is accidentally observed as being “up” (e.g. because of measurement noise). If the TF element is “present”, then with a probability of 0.70 the target gene is considered “up”, and with a probability of 0.30 the target gene is considered “down”. The latter values are chosen this way, because there can be several causes why a target gene is not highly expressed even though the TF element is present, e.g., because the gene's promoter region is methylated. In the case that a target gene is not up-regulated by the TF element, but down-regulated, the probabilities are chosen in a similar way, but reflecting the down-regulation upon presence of the TF element. The parameters describing the relationships between (ii) the target genes and their respective probesets have been calibrated on experimental data. For the latter, in this example, microarray data was used from patients samples which are known to have an active PR cellular signaling pathway whereas normal, healthy samples from a different data set were used as passive PR cellular signaling pathway samples, but this could also be performed using cell line experiments or other patient samples with known cellular signaling pathway activity status. The resulting conditional probability tables are given by:

A: for upregulated target genes

| | PS_{i,j} = low | PS_{i,j} = high |
|---|---|---|
| TGᵢ = down | $\frac{{AL}_{i , j} + 1}{{AL}_{i , j} + {AH}_{i , j} + 2}$ | $\frac{{AH}_{i , j} + 1}{{AL}_{i , j} + {AH}_{i , j} + 2}$ |
| TGᵢ = up | $\frac{{PL}_{i , j} + 1}{{PL}_{i , j} + {PH}_{i , j} + 2}$ | $\frac{{PH}_{i , j} + 1}{{PL}_{i , j} + {PH}_{i , j} + 2}$ |

B: for downregulated target genes

| | PS_{i,j} = low | PS_{i,j} = high |
|---|---|---|
| TGᵢ = down | $\frac{{PL}_{i , j} + 1}{{PL}_{i , j} + {PH}_{i , j} + 2}$ | $\frac{{PH}_{i , j} + 1}{{PL}_{i , j} + {PH}_{i , j} + 2}$ |
| TGᵢ = up | $\frac{{AL}_{i , j} + 1}{{AL}_{i , j} + {AH}_{i , j} + 2}$ | $\frac{{AH}_{i , j} + 1}{{AL}_{i , j} + {AH}_{i , j} + 2}$ |

In these tables, the variables AL_{i,j}, AH_{i,j}, PL_{i,j}, and PH_{i,j} indicate the number of calibration samples with an “absent” (A) or “present” (P) transcription complex that have a “low” (L) or “high” (H) probeset intensity, respectively. Dummy counts have been added to avoid extreme probabilities of 0 and 1.

To discretize the observed probeset intensities, for each probeset PS_{i,j} a threshold t_{i,j} was used, below which the observation is called “low”, and above which it is called “high”. This threshold has been chosen to be the (weighted) median intensity of the probeset in the used calibration data set. Due to the noisiness of microarray data, a fuzzy method was used when comparing an observed probeset intensity to its threshold, by assuming a normal distribution with a standard deviation of 0.25 (on a log 2 scale) around the reported intensity, and determining the probability mass below and above the threshold.

If instead of the exemplary Bayesian network described above, a (pseudo-) linear model as described in section 1 above was employed, the weights indicating the sign and magnitude of the correlation between the nodes and a threshold to call whether a node is either “absent” or “present” would need to be determined before the model could be used to infer cellular signaling pathway activity in a test sample. One could use expert knowledge to fill in the weights and the threshold a priori, but typically the model would be trained using a representative set of training samples, of which preferably the ground truth is known, e.g., expression data of probesets in samples with a known “present” transcription factor complex (=active cellular signaling pathway) or “absent” transcription factor complex (=passive cellular signaling pathway).

Known in the field are a multitude of training algorithms (e.g., regression) that take into account the model topology and changes the model parameters, here, the weights and the threshold, such that the model output, here, a weighted linear score, is optimized. Alternatively, it is also possible to calculate the weights directly from the observed expression levels without the need of an optimization algorithm.

A first method, named “black and white”-method herein, boils down to a ternary system, in which each weight is an element of the set {−1, 0, 1}. If this is put in a biological context, the −1 and 1 correspond to target genes or probesets that are down- and up-regulated in case of cellular signaling pathway activity, respectively. In case a probeset or target gene cannot be statistically proven to be either up- or down-regulated, it receives a weight of 0. In one example, a left-sided and right-sided, two sample t-test of the expression levels of the active cellular signaling pathway samples versus the expression levels of the samples with a passive cellular signaling pathway can be used to determine whether a probe or gene is up- or down-regulated given the used training data. In cases where the average of the active samples is statistically larger than the passive samples, i.e., the p-value is below a certain threshold, e.g., 0.3, the target gene or probeset is determined to be up-regulated. Conversely, in cases where the average of the active samples is statistically lower than the passive samples, the target gene or probeset is determined to be down-regulated upon activation of the cellular signaling pathway. In case the lowest p-value (left- or right-sided) exceeds the aforementioned threshold, the weight of the target gene or probeset can be defined to be 0.

A second method, named “log odds”-weights herein, is based on the logarithm (e.g., base e) of the odds ratio. The odds ratio for each target gene or probeset is calculated based on the number of positive and negative training samples for which the probeset/target gene level is above and below a corresponding threshold, e.g., the (weighted) median of all training samples. A pseudo-count can be added to circumvent divisions by zero. A further refinement is to count the samples above/below the threshold in a somewhat more probabilistic manner, by assuming that the probeset/target gene levels are e.g. normally distributed around its observed value with a certain specified standard deviation (e.g., 0.25 on a 2-log scale), and counting the probability mass above and below the threshold. Herein, an odds ratio calculated in combination with a pseudo-count and using probability masses instead of deterministic measurement values is called a “soft” odds ratio.

Further details regarding the inferring of cellular signaling pathway activity using mathematical modeling of target gene expression can be found in Verhaegh W. et al., “Selection of personalized patient therapy through the use of knowledge-based computational models that identify tumor-driving signal transduction pathways”, Cancer Research, Vol. 74, No. 11, 2014, pages 2936 to 2945.

### 4. Experimental Results

To demonstrate the PR models utility, here we present results obtained by applying various calibrated PR models to a series of Affymetrix HGU133Plus2.0 data sets. The models are identified by their target gene list and calibration set. For example, PR-A_EmLPxHS is the PR-A specific model calibrated with the EmLPxHS calibration set (see Table 4) that uses the PR-A specific target gene list. Activity read-outs are either presented as odds on:off (the odds of being active vs. being inactive), represented in a base 2 log scale, or as a normalized activity obtained by normalizing the log 2(odds on:off) to values between 0 and 100. The later normalized activity scale is useful when comparing activities of two different models that use a different number of target genes, target probesets, or calibration set, since the range of the log 2(odds on:off) of a model is highly dependent on those values.

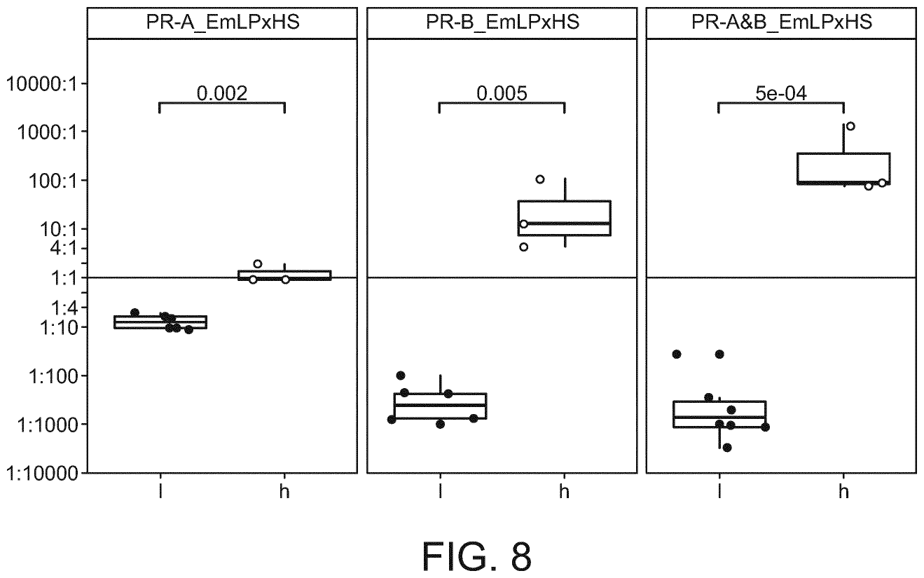

FIG. 8 shows PR cellular signaling pathway activity predictions of trained Bayesian network models on QC pass normal endometrium samples from GSE29921. Samples were taken at different days of the menstrual cycle and the progesterone level is available. Left: PR-A (PR-A_EmLPxHS) model; middle: PR-B (PR-B_EmLPxHS) model; right: PR-A&B (PR-A&B_EmLPxHS) model. PR cellular signaling pathway activity levels (shown as odds PR on:off) are significantly lower in samples with a low progesterone (“1”) concentration (av=0.16 ng/mL, sd=0.11 ng/mL, n=6) than in samples with a high progesterone (“h”) concentration (av=10.2 ng/mL, sd=3.5 ng/mL, n=3). Correlations between progesterone levels and PR cellular signaling pathway activity are 0.97, 0.97, and 0.96 for the PR-A, PR-B and PR-A&B models, respectively. (All samples in the EmLPxHS calibration set.)

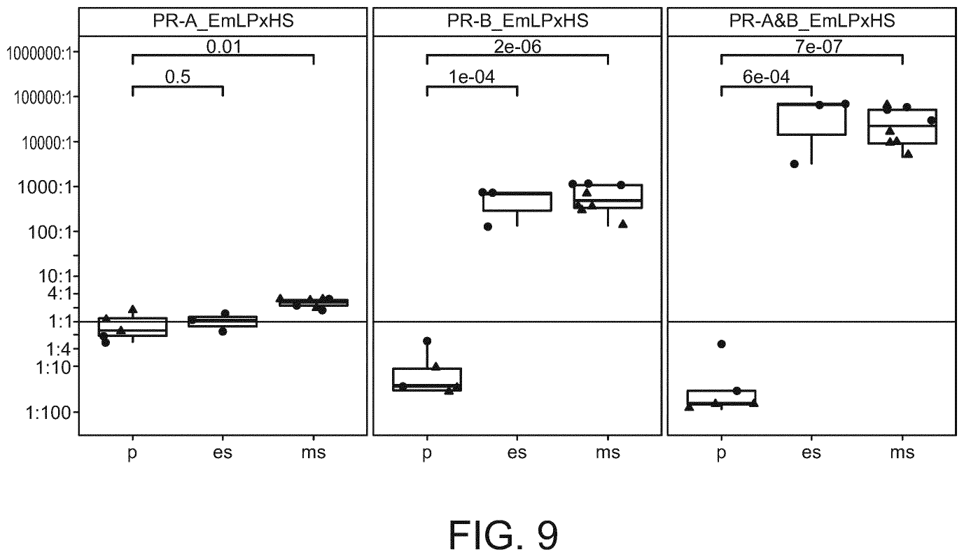

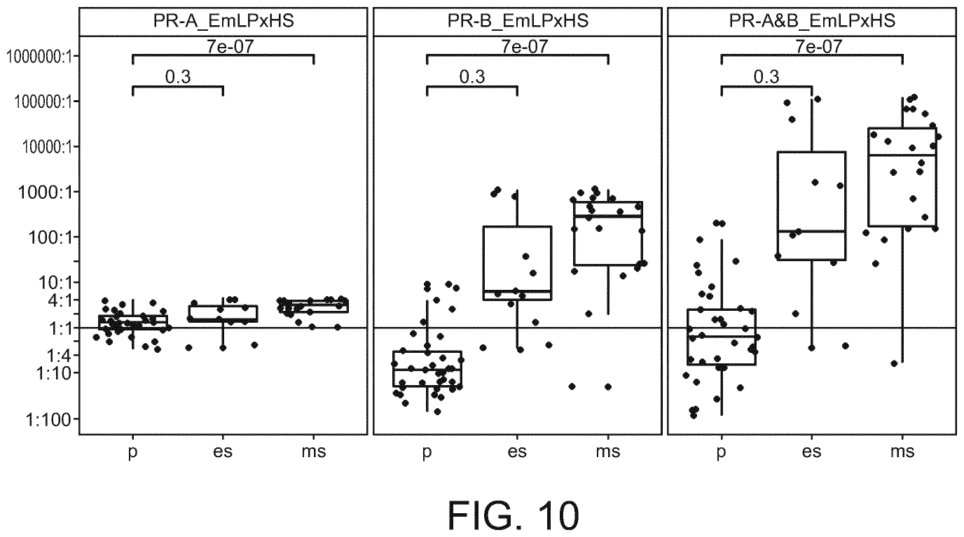

FIGS. 9 and 10 show PR cellular signaling pathway activity predictions of trained Bayesian network models on QC pass normal endometrium samples from GSE6364 and GSE51981, respectively. The samples were taken at different phases of the menstrual cycle. Left: PR-A (PR-A_EmLPxHS) model; middle PR-B (PR-B_EmLPxHS) model; right: PR-A&B (PR-A&B_EmLPxHS) model. PR cellular signaling pathway activity levels (shown as odds PR on:off) are significantly higher in the mid secretory phase (“ms”) than in the proliferative phase (“p”). Except for PR-A, PR cellular signaling pathway activity levels are also significantly higher in the early secretory phase (“es”) than in the proliferative phase. (The triangles show samples in the EmLPxHS calibration set.)

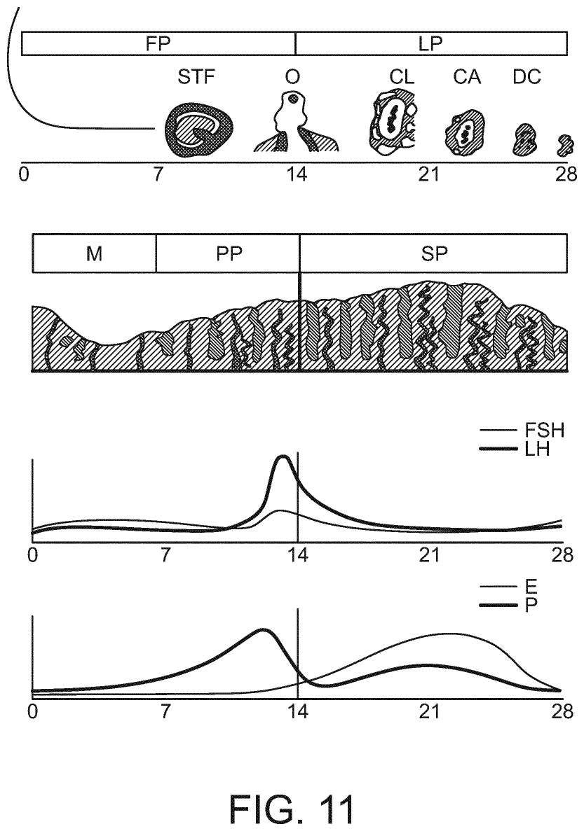

FIG. 11 shows the progression of the menstrual cycle and the different hormones contributing to it. The menstrual cycle consists of a proliferative (1-2 weeks) and a secretory phase (2 weeks) which is followed by menstruation. During the proliferative phase the estradiol level increases and then peaks high, just prior to ovulation. Then the luteal or secretory phase starts, during which progesterone levels increases, and estradiol levels are lower. As can be seen, the ovarian cycle phases consist of the follicular phase (“FP”) and the luteal phase (“LP”). The cycle is required for the production of oocytes, and for the preparation of the uterus for pregnancy. The menstrual cycle occurs due to the rise and fall of hormones. This cycle results in the thickening of the lining of the uterus, and the growth of an egg, (which is required for pregnancy). The egg is released from an ovary around day fourteen in the cycle; the thickened lining of the uterus provides nutrients to an embryo after implantation. If pregnancy does not occur, the lining is released in what is known as menstruation. Abbreviations used in the figure: Selected tertiary follicle (“STF”); Ovluation (“0”); Corpus luteuni (“CL”); Corpus ablicans (“CA”); Degrading corpus (“DC”); Menses (“M”); Proliferative phase (“PP”); Secretory phase (“SP”). The two plots at the bottom of the figure show the hormone levels during the different days of the menstrual cycle. Upper plot: Pituitary hormon levels (FSH; LH). Lower plot: Ovarian hormon levels (estrogen (“E”); progesterone (“P”)).

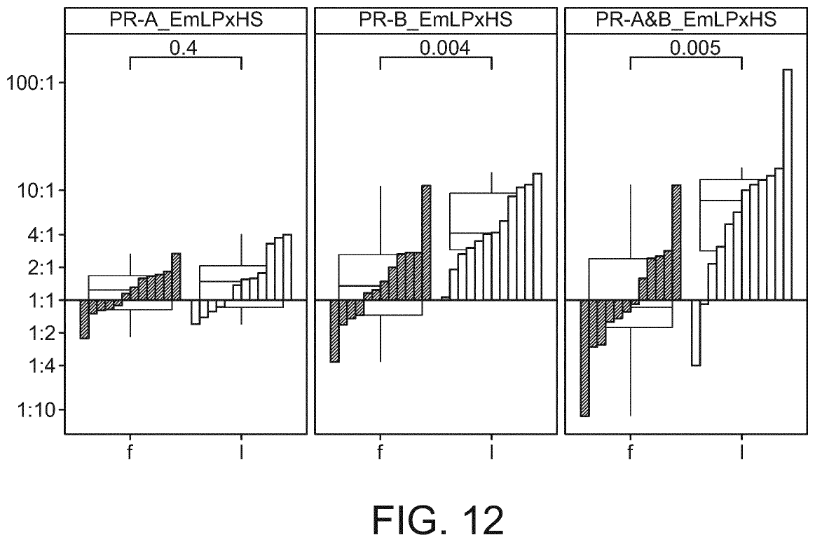

FIG. 12 shows PR cellular signaling pathway activity predictions of trained Bayesian network models on fallopian tube samples from GSE10971. Samples were taken at different phases of the ovulation cycle. Left: PR-A (PR-A_EmLPxHS) model; middle: PR-B (PR-B_EmLPxHS) model; right: PR-A&B (PR-A&B_EmLPxHS) model. Mean PR cellular signaling pathway activity levels (shown as odds PR on:off) are higher in the luteal phase (“1”) compared to the follicular phase (“f”).

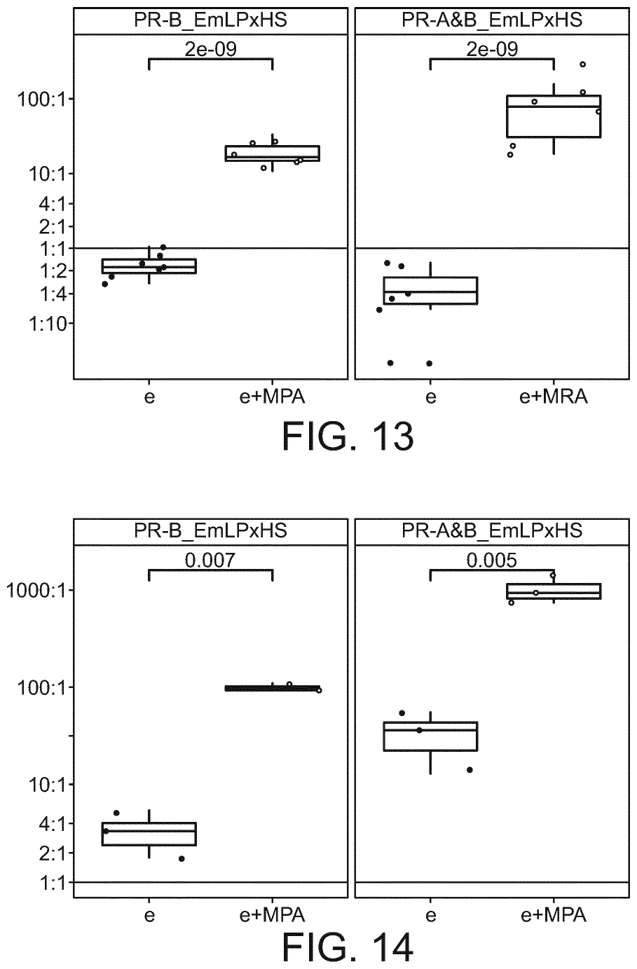

FIG. 13 shows PR cellular signaling pathway activity predictions of trained Bayesian network models on QC pass menopausal normal endometrium samples from GSE12446. Patients were treated with estradiol (“e”) or estradiol+MPA (“e+MPA”) for 21 days prior to surgery. Left: PR-B (PR-B_EmLPxHS) model; right: PR-A&B (PR-A&B_EmLPxHS) model. PR cellular signaling activity levels (shown as odds PR on:off) are significantly higher in estradiol+MPA treated samples than in estradiol only treated samples.

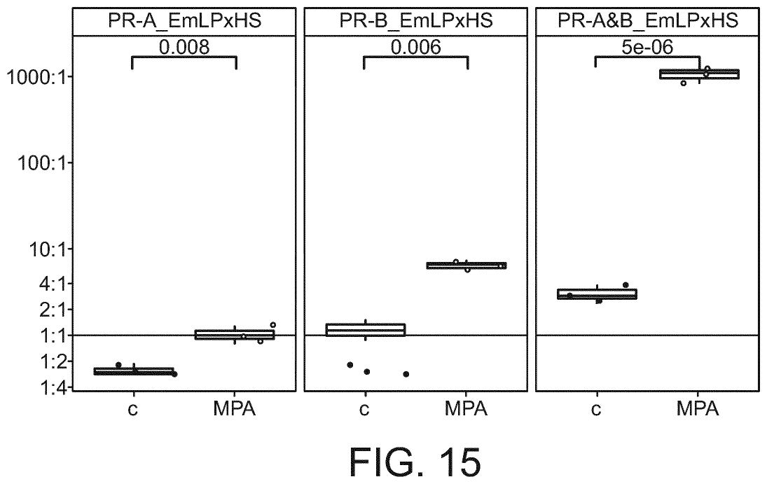

FIG. 14 shows PR cellular signaling pathway activity predictions of trained Bayesian network models on QC pass term decidual cells (from normal placentae) samples from GSE65835. Cells were cultured with estradiol (“e”) or estradiol+MPA (“e+MPA”). Left: PR-B (PR-B_EmLPxHS) model; right: PR-A&B (PR-A&B_EmLPxHS) model. PR cellular signaling pathway activity levels (shown as odds PR on:off) are significantly higher in estradiol+MPA treated samples than in estradiol only treated samples.

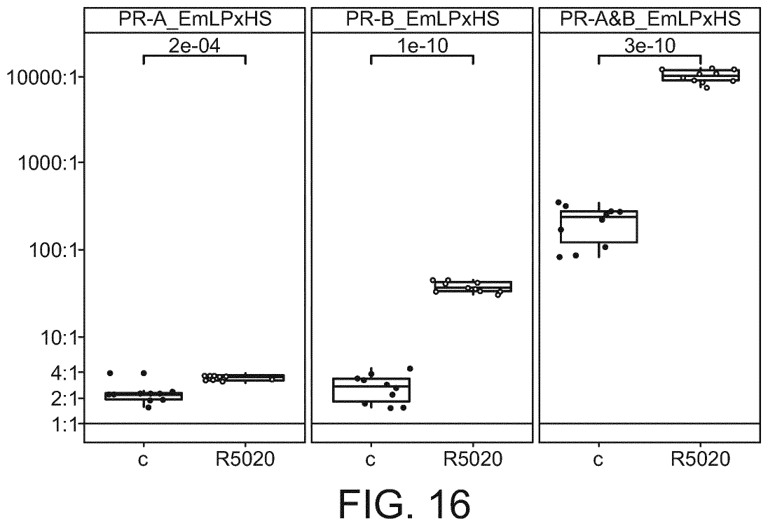

FIG. 15 shows PR cellular signaling pathway activity predictions of trained Bayesian network models on QC pass PR-A and PR-B expressing ishikawa IKPR-AB36 endometrium cell line samples from GSE29435. Cells were cultured for 48h in the presence or absence of 1 nM MPA. Left: PR-A (PR-A_EmLPxHS) model; middle: PR-B (PR-B_EmLPxHS) model; right: PR-A&B (PR-A&B_EmLPxHS) model. PR cellular signaling pathway activity levels (shown as odds PR on:off) are significantly higher in MPA treated samples than in control (“c”) treated samples.

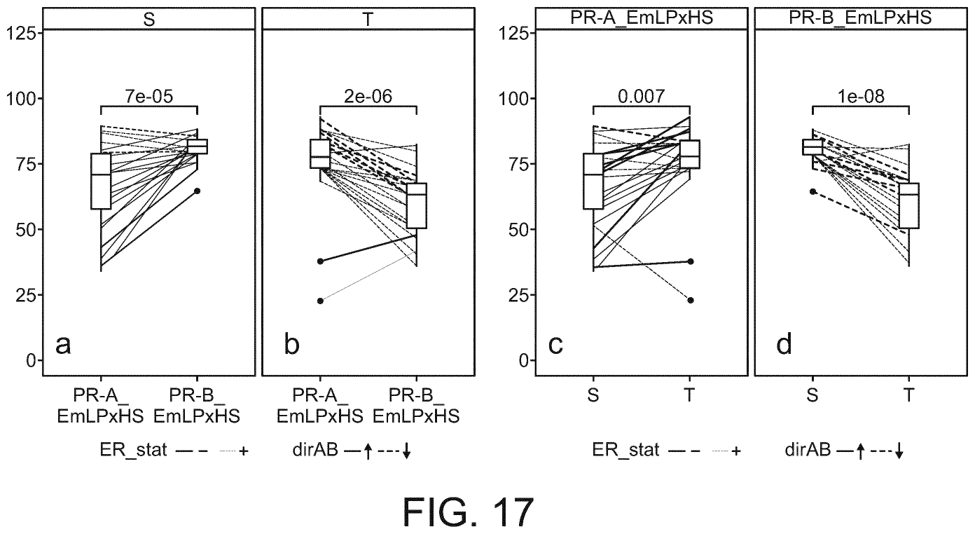

FIG. 16 shows PR cellular signaling pathway activity predictions of trained Bayesian network models on QC pass PR-B expressing hMEC breast cell line samples from GSE24468. Cells were cultured for 16h in the presence or absence of 10 nM R5020. Left: PR-A (PR-A_EmLPxHS) model; middle: PR-B (PR-B_EmLPxHS) model; right: PR-A&B (PR-A&B_EmLPxHS) model. PR cellular signaling pathway activity levels are significantly higher in R5020 treated samples than in control (“c”) treated samples.

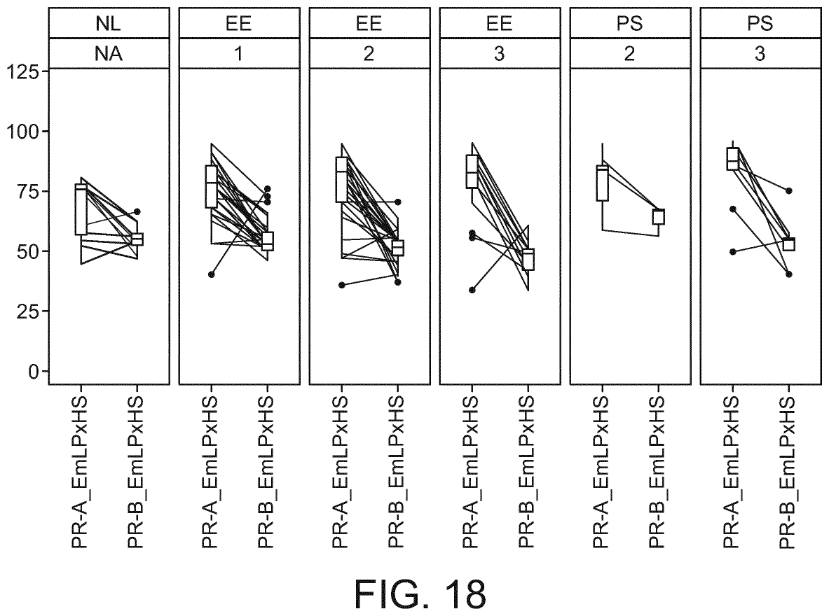

FIG. 17 shows PR cellular signaling pathway activity predictions of trained Bayesian network models on QC pass paired normal vs. breast cancer samples from GSE10810. Left: Difference between PR-A and PR-B cellular signaling pathway activity in adjacent normal (“S”) and tumor (“T”) samples. Right: Difference in PR-A and PR-B cellular signaling pathway activity between paired adjacent normal (“S”) and tumor (“T”) samples. Normalized activities are used for a better comparison of the PR-A and PR-B models. Compared to normal tissue, PR-A and PR-B cellular signaling pathway activity levels are disrupted in breast cancer tissue. While normalized PR-A and PR-B cellular signaling pathway activities are similar or PR-B is higher than PR-A in normal issue (see “a”), normalized PR-B cellular signaling pathway activity is generally lower than normalized PR-A cellular signaling pathway activity in tumor tissue (see “b”). While normalized PR-A cellular signaling pathway activity is generally higher in tumor than in normal samples (see “c”), PR-B is generally lower in tumor than in normal samples (see “d”).

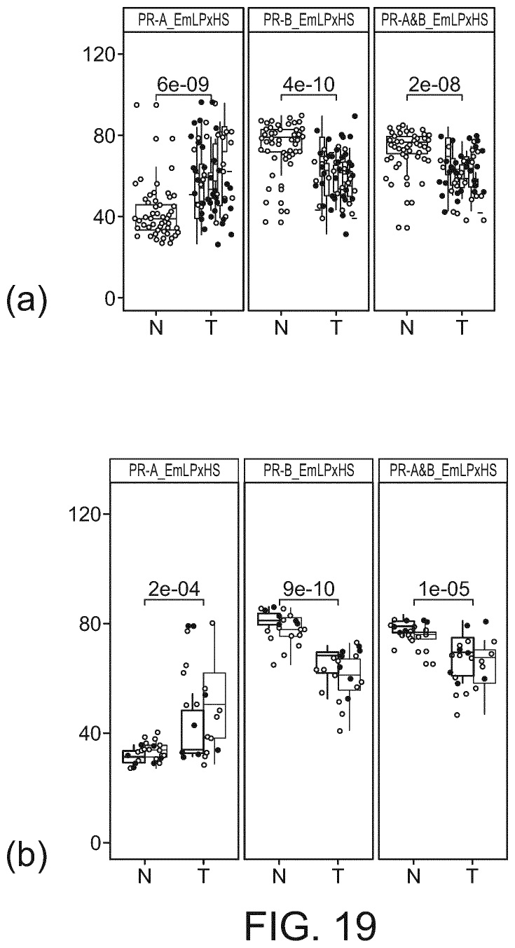

FIG. 18 shows PR cellular signaling pathway activity predictions of trained Bayesian network models on normal vs. endometrial cancer samples from GSE17025. From left to right: Normal samples (“NL”), Endometrioid cancer grades 1 to 3 (“EE 1-2-3”), Papillary serous cancer grades 2 and 3 (“PS 2-3”). Normalized PR-A cellular signaling pathway activity is increased in endometrial cancer samples compared to normal samples and normalized PR-B cellular signaling pathway activity is similar or decreased in endometrial cancer samples compared to normal samples. The difference between PR-A and PR-B increases with cancer grade.

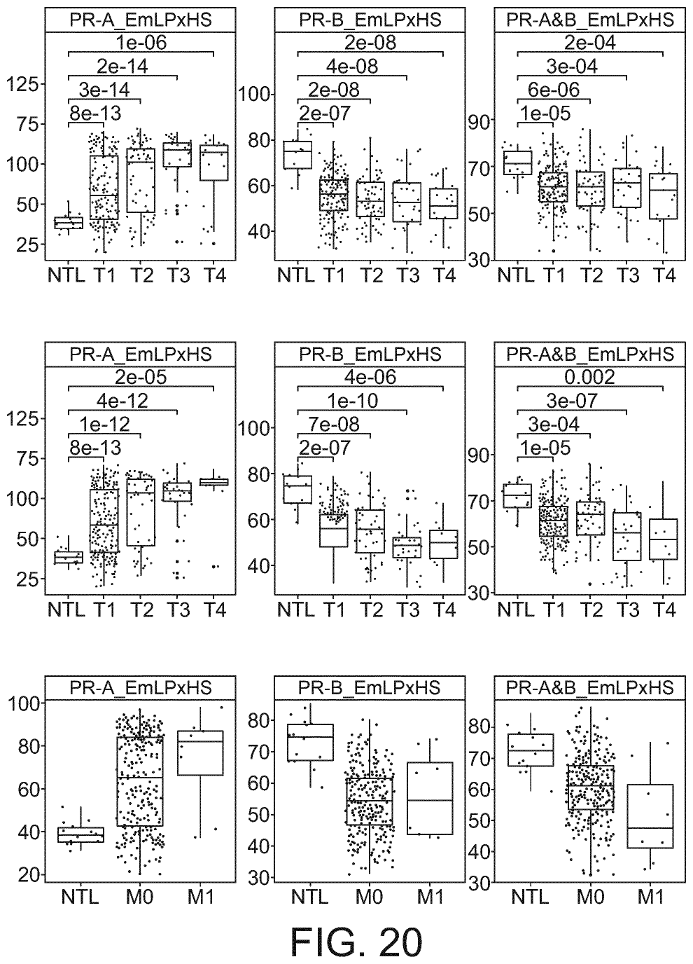

FIG. 19 shows PR cellular signaling pathway activity predictions of trained Bayesian network models on paired normal vs. tumor lung cancer samples. Left: Samples from non-smoking female lung cancer in Taiwan from GSE19804; right: Samples from stage I lung adenocarcinoma from GSE27626. Compared to normal lung (“N”), normalized PR-A and PR-B cellular signaling pathway activity levels are disrupted in cancer (“T”). While normalized PR-A cellular signaling pathway activity levels increase significantly in cancer compared to normal tissue, normalized PR-B and PR-A&B cellular signaling pathway activity levels are significantly reduced from normal to cancer. Cancer samples are grouped by tumor stage. Disruption in normalized PR-A and PR-B cellular signaling pathway activity levels are larger on more severe stages.

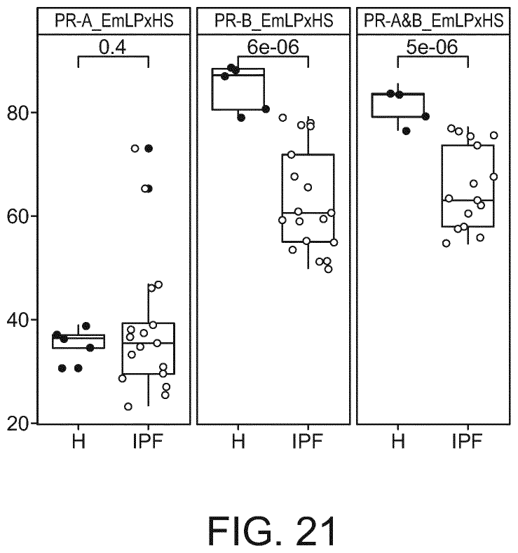

FIG. 20 shows PR cellular signaling pathway activity predictions of trained Bayesian network models on normal and tumor lung cancer samples from GSE30219. Left: PR-A (PR-A_EmLPxHS) model; middle: PR-B (PR-B_EmLPxHS) model; right: PR-A&B (PR-A&B_EmLPxHS). Cancer samples are grouped by TNM tumor stage from top to bottom. Compared to normal lung (“NTL”), normalized PR-A and PR-B cellular signaling pathway activity levels are disrupted in cancer (“N0” to “N3”). While normalized PR-A cellular signaling pathway activity levels increase significantly in cancer compared to normal tissue, normalized PR-B and PR-A&B cellular signaling pathway activity levels are significantly reduced from normal to cancer. Disruption in normalized PR-A and PR-B cellular signaling pathway activity levels are larger on more severe stages (mostly due to further increase in PR-A cellular signaling pathway activity levels).

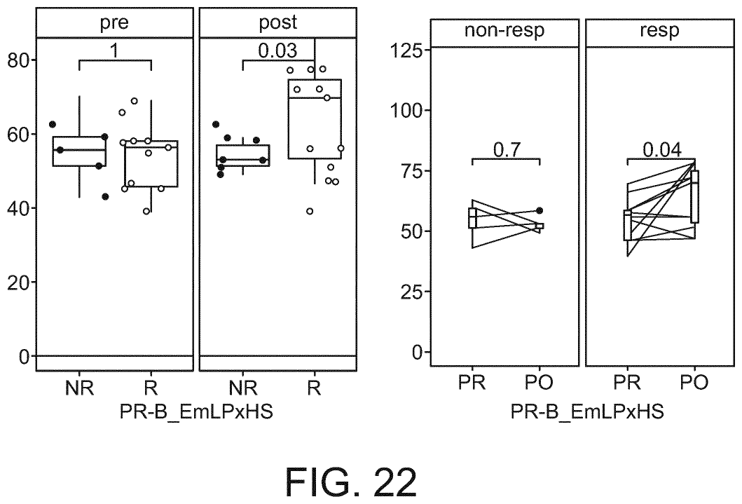

FIG. 21 shows PR cellular signaling pathway activity predictions of trained Bayesian network models on normal vs. idiopathic pulmonary fibrosis (IPF) samples from GSE24206. Compared to normal lung (“H”), normalized PR-A and PR-B cellular signaling pathway activity levels are disrupted in IPF. While normalized PR-A cellular signaling pathway activity levels remain similar in normal and IPF, normalized PR-B and PR-A&B cellular signaling pathway activity levels are significantly reduced from normal to IPF.

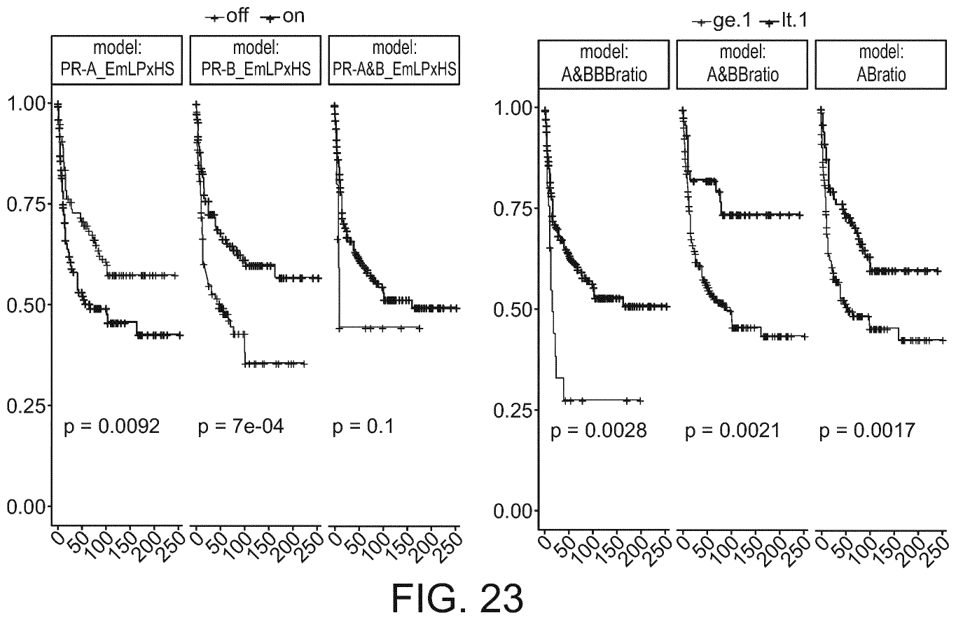

FIG. 22 shows PR-B cellular signaling pathway activity predictions of trained Bayesian network models on QC pass paired pre/post letrozole treatment breast cancer samples from GSE10281. ER positive breast cancer patients were treated for 3 months with the aromase inhibitor letrozole in a neoadjuvant setting. Left: Normalized PR-B cellular signaling pathway activity difference in samples from non-responsive (“NR”) vs. responsive (“R”) patients in pre-treatment samples (“pre”) and post-treatment samples (“post”); right: Normalized PR-B cellular signaling pathway activity difference in paired pre vs. post treatetment samples in non-responders and in responders. After treatment, mean normalized PR-B cellular signaling pathway activity is higher in responders than in non-responders. Mean normalized PR-B cellular signaling pathway activity goes up in responders with activity levels approaching PR-B activity in normal samples (see FIG. 17). Normalized PR-B cellular signaling pathway activity levels remain similar to pre-treatment in non-responders.

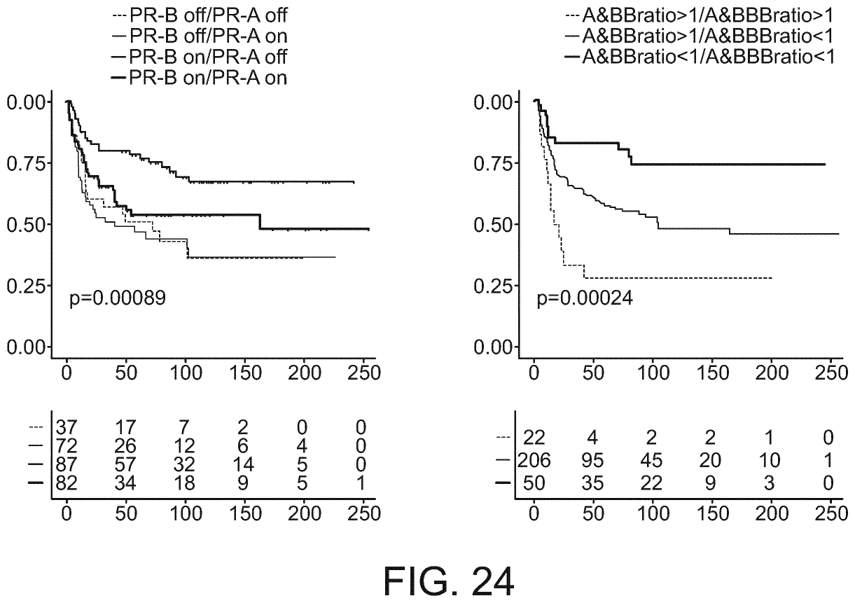

FIG. 23 shows PR cellular signaling pathway activity predictions of trained Bayesian network models as a prognostic risk factor for the progression of lung cancer on QC pass samples from GSE30219. Left: PR-A, PR-B and PR-A&B were discretized between “on” and “off” (probability of being active ≥50% and <50%, respectively); right: Ratios between (1) normalized PR-A and normalized PR-B (ABratio), (2) normalized PR-A&B and normalized PR-B (A&BBratio) and (3) normalized A&BBratio and normalized PR-B (A&BBBratio) were computed and discretized between “ge.1” and “lt.1” (ratio is ≥1 and <1, respectively). Survival curves show (in time to relapse) that, besides for the PR-A&B model (PR-A&B_EmLHxHS), the PR cellular signaling pathway activity predictions are significant prognostic factors for the progression of lung cancer.

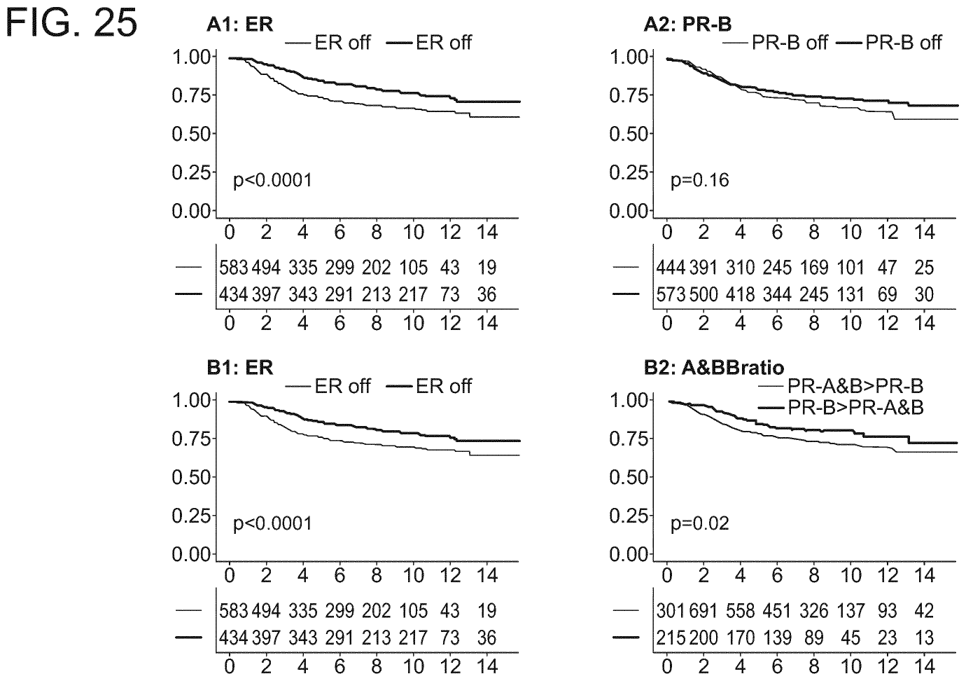

FIG. 24 shows combined PR cellular signaling pathway activities predictions of trained Bayesian network models as a prognostic risk factor for the progression of lung cancer on QC pass samples from GSE30219. PR-A, PR-B and PR-A&B were discretized between “on” and “off” (probability of being active ≥50% and <50%, respectively). Ratios between (1) normalized PR-A&B and normalized PR-B (A&BBratio) and (2) normalized A&BBratio and normalized PR-B (A&BBBratio) were computed and discretized between “ge.1” and “lt.1” (ratio is ≥1 and <1, respectively). “>1” and “<1” (ratio is ≥1 and <1, respectively). Left: combination of PR-A and PR-B. Right: combination of A&BBratio and A&BBBratio. Survival curves show (in survival probability) that both combinations are significant prognostic factors in progression of lung cancer. The PR-B off group has the shortest time to progression independent of PR-A. PR-B on/PR-A off has the longest time to progression and PR-B on/PR-A on has an intermediate time to progression. A&BBratio>1/A&BBBratio>1 group have the shortest time to progression, while A&BBratio<1/A&BBBratio<1 groups have the longest time to progression.

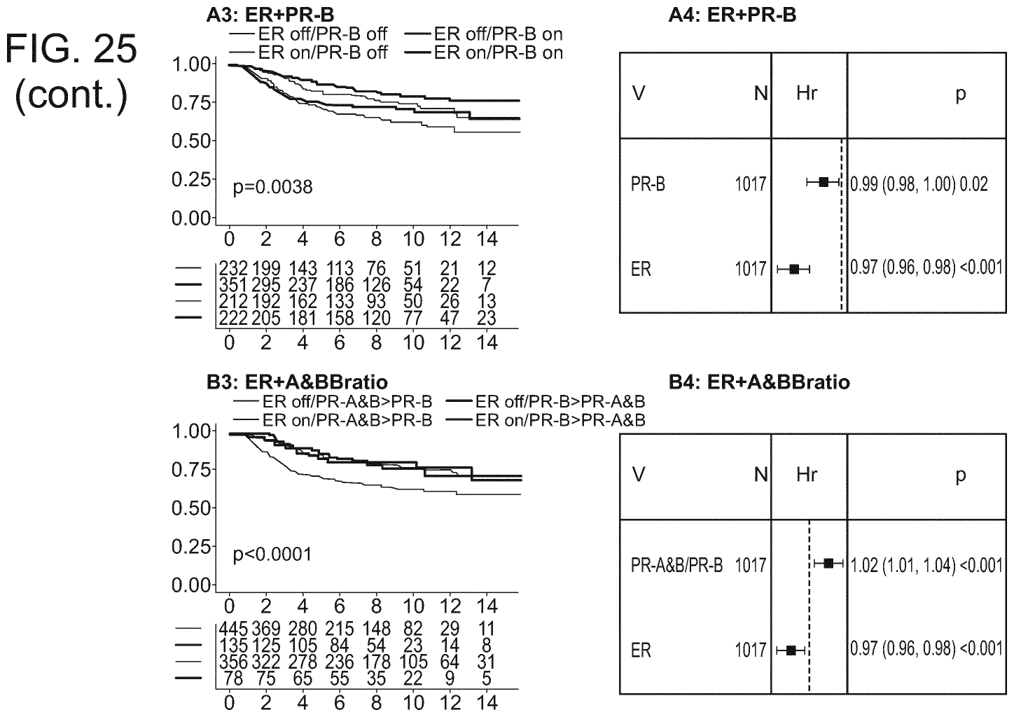

FIG. 25 shows PR cellular signaling pathway activity predictions of trained Bayesian network models as a prognostic risk factor for the progression of breast cancer on QC pass samples from GSE6532, GSE9195, GSE21653, GSE20685, GSE58812, and EMTAB365. ER (ER_E01MCF7g28 previously described), PR-B (PR-B_EmLHxHS) and PR-A&B (PR-A&B_EmLHxHS) were discretized between “on” and “off” (probability of being active ≥50% and <50%, respectively). A&BBratio (ratio between normalized PR-A&B and normalized PR-B) was discretized between “A&B>B” and “B>A&B” (ratio is ≥1 and <1, respectively). A1 to A3: survival curves of discretized ER, PR-B, and ER+PR-B, respectively. A4: summary of multivariate cox proportional hazard regression on the continuous ER+PR-B model. B1 to B3: survival curves of discretized ER, A&BBratio, and ER+A&BBratio, respectively. B4: summary of multivariate cox proportional hazard regression on the continuous ER+A&BBratio. A&BBratio in the survival curves and cox proportional hazard regression exemplifies the added value adding PR-B or A&BBratio to ER in a prognostic cancer model.

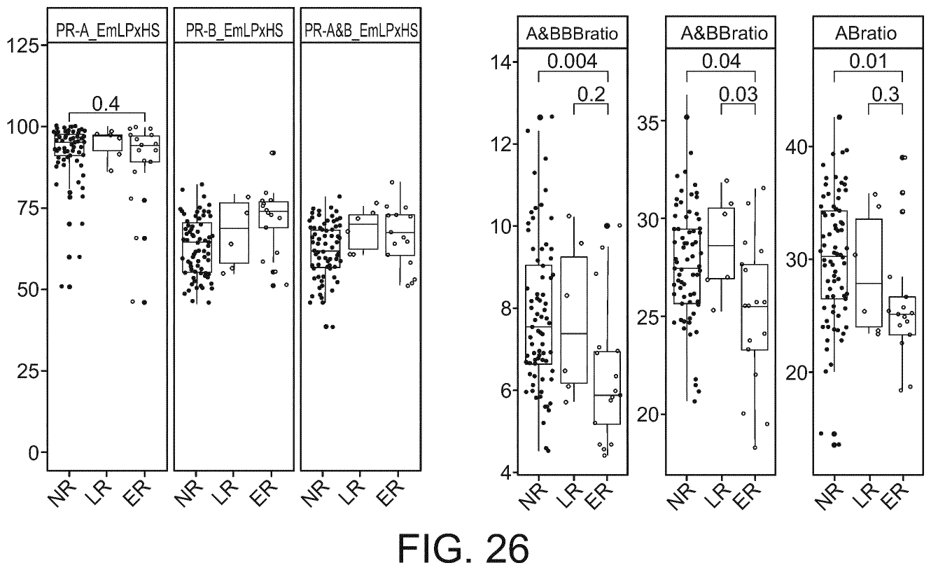

FIG. 26 shows PR cellular signaling pathway activity predictions of trained Bayesian network models as a prognostic risk factor for the progression of pediatric acute lymphoblastic leukemia (ALL) on QC pass samples from GSE13576. Ratios between (1) normalized PR-A and normalized PR-B (ABratio), (2) normalized PR-A&B and normalized PR-B (A&BBratio) and (3) normalized A&BBratio and normalized PR-B (A&BBBratio) were computed and normalized. Left: normalized PR-A, PR-B and PR-A&B pathway activities. Right: PR-B and all 3 ratios are significantly higher in early relapse (“ER”) compared to controls (“NR”), A&BBratio is also significantly higher in early relapse compared to late relapse (“LR”).

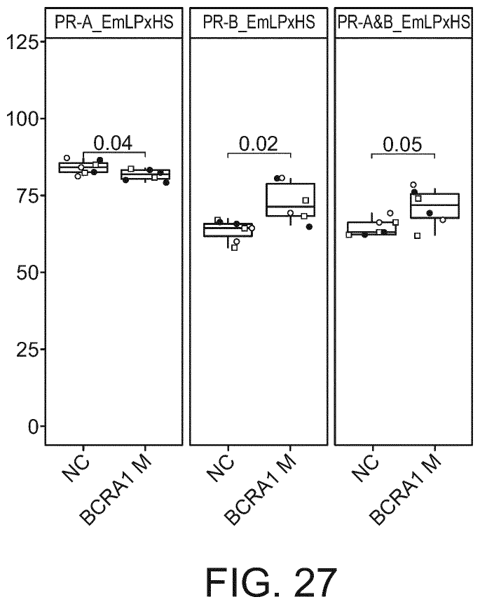

FIG. 27 shows PR cellular signaling pathway activity predictions of trained Bayesian network models as a prognostic risk factor in breast cancer on primary normal breast cell cultures samples from GSE13671. Compared to cultures form normal non-mutated breast tissue (“NC”), PR-A and PR-B cellular signaling pathway activity levels are disrupted in cultures from BCRA1 mutated tissue (“BCRA1 M”). While PR-A cellular signaling pathway activity levels are significantly lower in mutated cultures, PR-B and PR-A&B cellular signaling pathway activity levels are significantly higher in mutated cultures. Different indicators represent different donors.

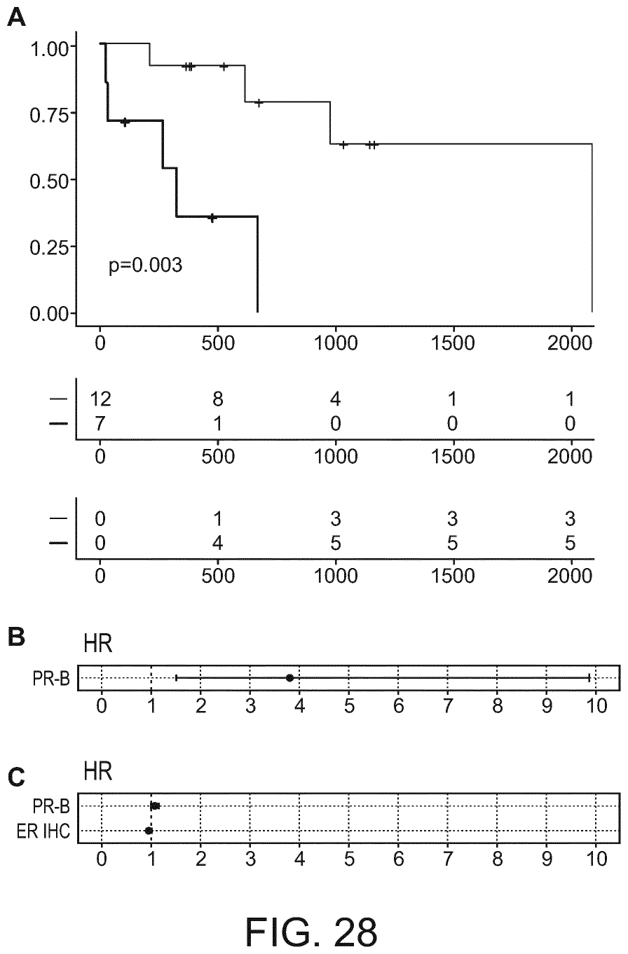

FIG. 28 shows PR cellular signaling pathway activity predictions of trained linear models as a predictor for progestogen therapy response in endometrial cancer. As an example, a linear PR-B model using only a shortlist of three target genes CCND1, FKBP5, and MYC (see also Table 10) based on PCR data was used. Normalized RT-qPCR of genes from the target gene shortlist were added and normalized to values between 0 and 10. PR cellular signaling pathway activity scores of the linear model were computed for pre-treatment endometrium samples of 19 patients that underwent subsequent progestogen hormone therapy. The linear model was discretized between high and low using the median as threshold and the graph shows the survival probability for low PR-B cellular signaling pathway activity (upper curve) and high PR-B cellular signaling pathway activity (lower curve). A) Survival curves for the discretized PR-B pathway activity. The numbers below the graphs show the number at risk and the cumulative number of events for the low PR-B cellular signaling pathway activity and the high PR-B cellular signaling pathway activity. B) Summary of univariate cox proportional hazard regression (number at risk) on the linear PR-B model using only the target genes CCND1, FKBP5, and MYC (number of samples=19; number of events=9, Hazard Ratio (HR)=3.8; Wald test p value=0.006). C) Summary of multivariate cox proportional hazard regression (cumulative number of events) on the linear PR-B model using only the target genes CCND1, FKBP5 and MYC and ER IHC (immunohistochemistry staining of nuclear ER, values normalized between 0 and 10) (number of samples=19 for both PR-B and ER IHC; number of events=9 for both PR-B and ER IHC, Hazard Ratio (HR)=1.12 for PR-B and 0.99 for ER IHC; Wald test p value=0.03 for PR-B and 0.3 for ER IHC).

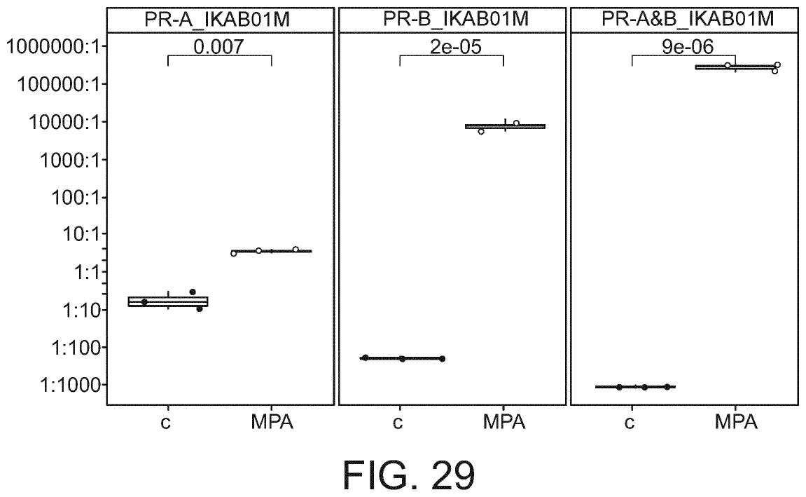

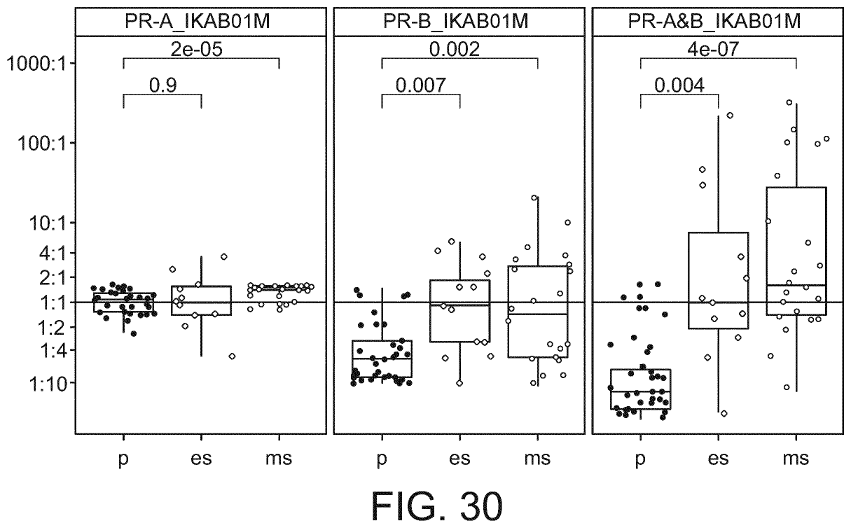

FIGS. 29 and 30 show PR cellular signaling pathway activity predictions of trained Bayesian network models calibrated with the alternative calibration set IKAB01M (IPR-AB36 endometrial cell line treated with vehicle control or 1 nM MPA; see Table 4) for the preferred PR-A, PR-B and PR-A&B target gene lists. FIG. 29: Inferred PR cellular signaling pathway activity levels on IKAB01M calibration samples from GSE29435 (see FIG. 15). FIG. 30: Normal endometrium during menstrual cycle QC pass samples from GSE51981 (see FIG. 10). The resulting PR cellular signaling pathway activity levels (shown as odds PR on:off) are similar to the results obtained with the preferred calibration set.

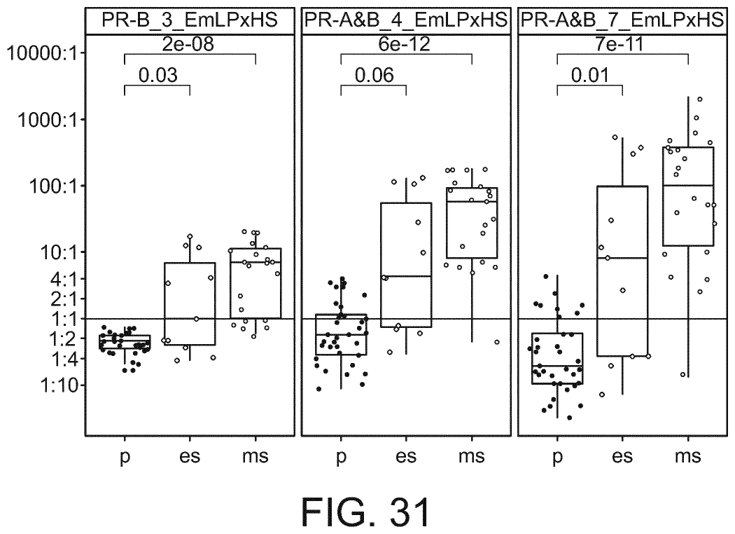

FIG. 31 shows PR cellular pathway activity predictions of trained Bayesian network models calibrated with two alternative target gene lists and the preferred calibration set EmLPxHS (see Table 4). The activities were inferred for normal endometrium during menstrual cycle QC pass samples from GSE51981 (see FIG. 10). Left: PR-B model using only a shortlist of three target genes CCND1, FKBP5, and MYC (PR-B_3_EmLPxHS; see also Table 10). Middle: PR-A&B model using only a shortlist of four target genes BCL6, CCND1, FKBP5, and MYC (PR-A&B_4_EmLPxHS; see also Table 13). Right: PR-A&B model using only a shortlist of seven target genes BCL6, CCND1, CDKN1A, FKBP5, MYC, SBK1, and VEGFA (PR-A&B_7_EmLPxHS; see also Table 14). The resulting PR cellular signaling pathway activities are substantially comparable to the results obtained with the preferred target gene lists (ses Tables 6 to 8).

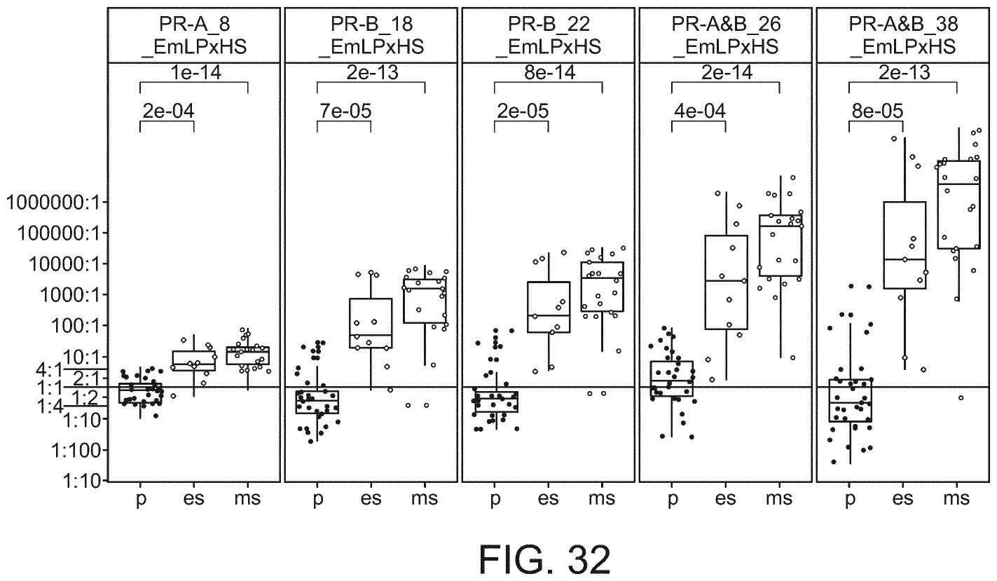

FIG. 32 shows PR cellular pathway activity predictions of trained Bayesian network models calibrated with five further alternative target gene lists and the preferred calibration set EmLPxHS (see Table 4). The activities were inferred for normal endometrium during menstrual cycle QC pass samples from GSE51981 (see FIG. 10). Left: PR-A model using a longlist of eight target genes BCL2L1, BIRC3, DDIT4, F3, MUC1, NEDD9, SGK1, and TRIM22 (PR-A_8_EmLPxHS; see also Table 9). Next, PR-B model using a longlist of eightteen target genes ARRDC1, ATP1B1, CCND1, CD82, E2F1, FKBP5, GOT1, HSD11B2, KANK1, MSX2, MYC, NET1, NFKBIA, PDK4, PLIN2, PTP4A2, SNTB2, and STAT5A (PR-B_18_EmLPxHS; see also Table 11). Next, PR-B model using a longlist of twentytwo target genes ARRDC1, ATP1B1, BIRC3, CCND1, CD82, DDIT4, E2F1, F3, FKBP5, GOT1, HSD11B2, KANK1, MSX2, MUC1, MYC, NET1, NFKBIA, PDK4, PLIN2, PTP4A2, SNTB2, and STAT5A (PR-B_22_EmLPxHS; see also Table 12). Next, PR-A&B model using a longlist of twentysix target genes AK4, ARRDC1, ATP1B1, BCL2L1, BCL6, BIRC3, CCND1, CD82, F3, FKBP5, GOT1, GRB10, HSD11B2, KLF4, MUC1, MYC, NEDD9, NET1, PDK4, PTP4A2, S100P, SGK1, SNTB2, STAT5A, TSC22D3, and VASP (PR-A&B_26_EmLPxHS; see also Table 15). Right, PR-A&B model using a longlist of thirtyeight target genes ABCG2, ACSS1, AK4, ARRDC1, ATP1B1, BCL2L1, BCL6, BIRC3, CCND1, CD82, CDKN1A, DDIT4, E2F1, F3, FKBP5, GOT1, GRB10, HPCAL1, HSD11B2, KANK1, KLF4, MSX2, MUC1, MYC, NEDD9, NET1, NFKBIA, PDK4, PLIN2, PTP4A2, S100P, SGK1, SNTB2, STAT5A, TRIM22, TSC22D3, VASP, and VEGFA (PR-A&B_38_EmLPxHS; see also Table 16). The resulting PR cellular signaling pathway activities are substantially comparable to the results obtained with the preferred target gene lists (ses Tables 6 to 8).

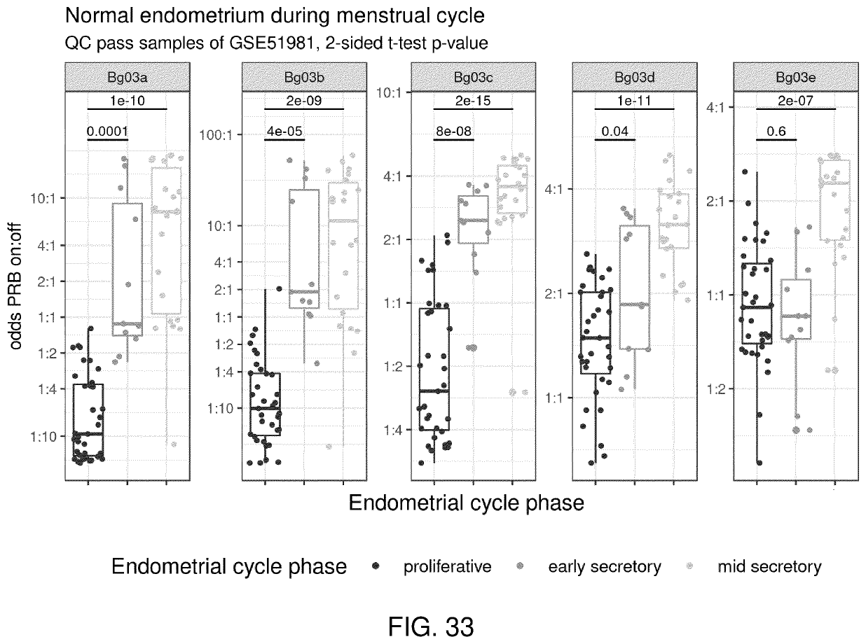

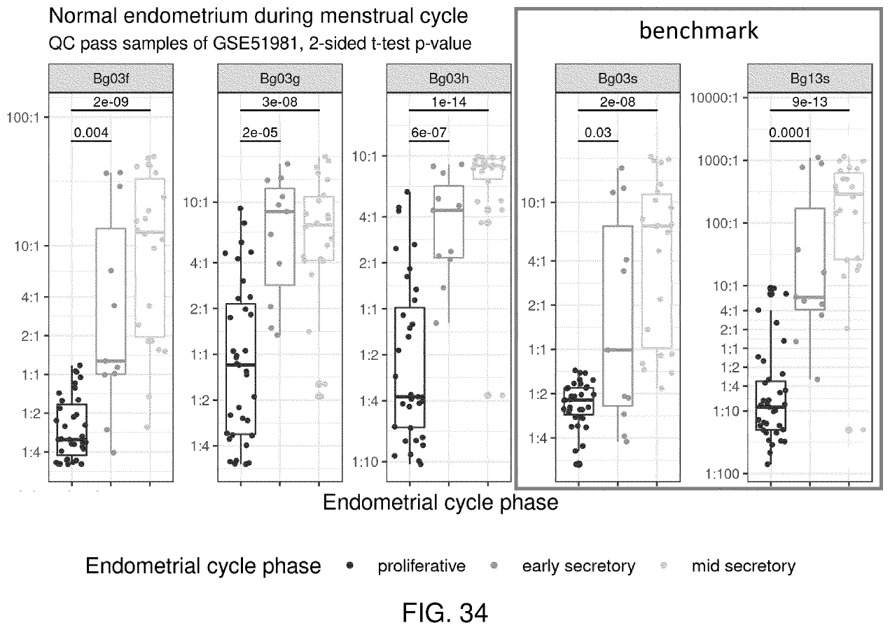

FIG. 33 shows PR-B cellular pathway activity predictions of trained Bayesian network models calibrated with alternative target gene lists comprising 3 randomly selected genes from the preferred PR-B target gene set (see Table 7), and the preferred calibration set EmLPxHS (see Table 4). The activities were inferred for normal endometrium during menstrual cycle QC pass samples from GSE51981 (see FIG. 10). From left to right the following gene sets are used: Bg03a (FKBP5, MSX2, NFKBIA), Bg03b (FKBP5, MSX2, KANK1), Bg03c (NET1, PDK4, MSX2), Bg03d (PDK4, CCND1, ARRDC1) and Bg03e (CCND1, HSD11B2, ATP1B1). The resulting PR-B cellular signaling pathway activities are substantially comparable to the results obtained with the preferred target gene lists (benchmark, see FIG. 34).

FIG. 34 shows PR-B cellular pathway activity predictions of trained Bayesian network models calibrated with alternative target gene lists comprising 3 randomly selected genes from the preferred PR-B target gene set (see Table 7), and the preferred calibration set EmLPxHS (see Table 4). The activities were inferred for normal endometrium during menstrual cycle QC pass samples from GSE51981 (see FIG. 10). From left to right the following gene sets are used: Bg03f(FKBP5, NET1, PLIN2), Bg03g (PDK4, KANK1, PLIN2), Bg03h(NET1, MSX2, PLIN2), Bg03s (all genes listed in Table 10) and Bg13 (all genes listed in Table 7). The resulting PR-B cellular signaling pathway activities are substantially comparable to the results obtained with the preferred target gene lists (benchmark).

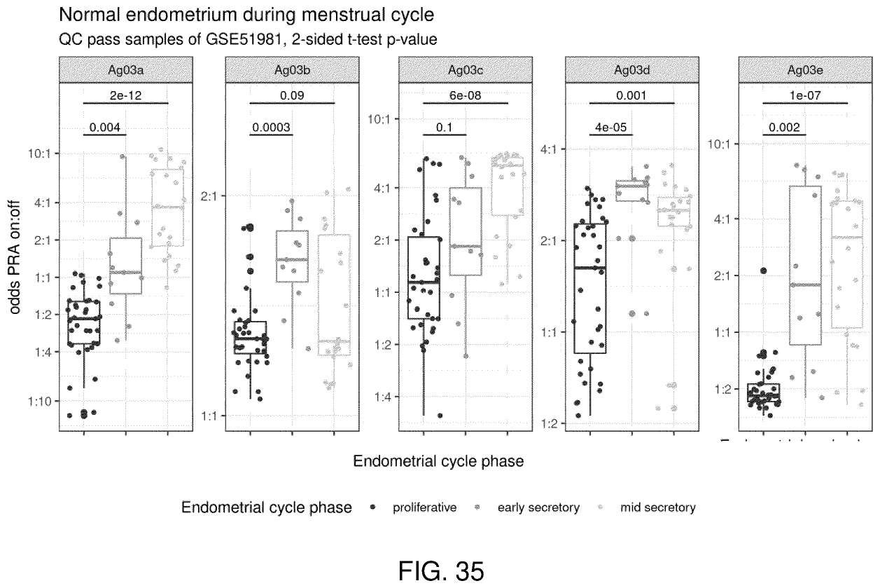

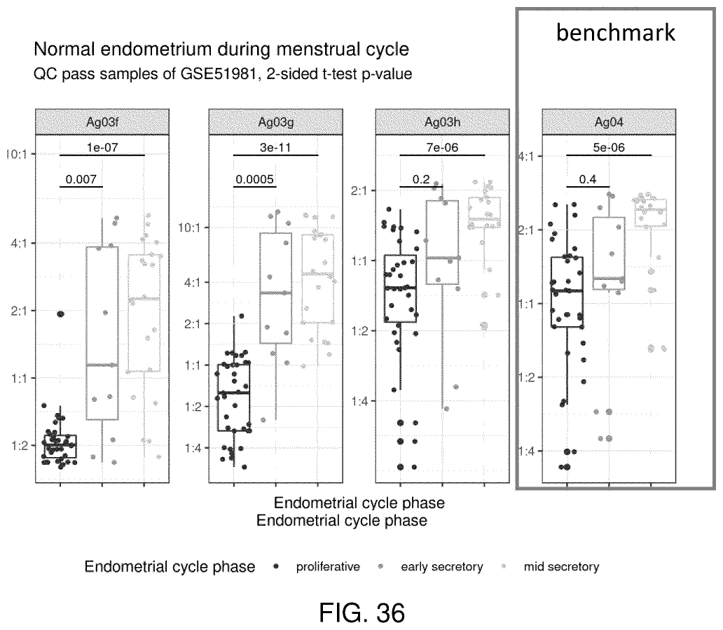

FIG. 35 shows PR-A cellular pathway activity predictions of trained Bayesian network models calibrated with alternative target gene lists comprising 3 randomly selected genes from the preferred PR-A target gene set (see Table 9), and the preferred calibration set EmLPxHS (see Table 4). The activities were inferred for normal endometrium during menstrual cycle QC pass samples from GSE51981 (see FIG. 10). From left to right the following gene sets are used: Ag03a (BCL2L1, NEDD9, SGK1), Ag03b, (BCL2L1, BIRC3, F3), Ag03c (BCL2L1, MUC1, NEDD9), Ag03d (MUC1, DDIT4, TRIM22) and Ag03e (BCL2L1, BIRC3, SGK1). The resulting PR-A cellular signaling pathway activities are substantially comparable to the results obtained with the preferred target gene lists (benchmark, see FIG. 36).

FIG. 36 shows PR-A cellular pathway activity predictions of trained Bayesian network models calibrated with alternative target gene lists comprising 3 randomly selected genes from the preferred PR-A target gene set (see Table 9), and the preferred calibration set EmLPxHS (see Table 4). The activities were inferred for normal endometrium during menstrual cycle QC pass samples from GSE51981 (see FIG. 10). From left to right the following gene sets are used: Ag03f (F3, SGK1, TRIM22), Ag03g (MUC1, SGK1, DDIT4), Ag03h (BCL2L1, BIRC3, F3) and Ag04 (all genes listed in Table 6). The resulting PR-A cellular signaling pathway activities are substantially comparable to the results obtained with the preferred target gene lists (benchmark).

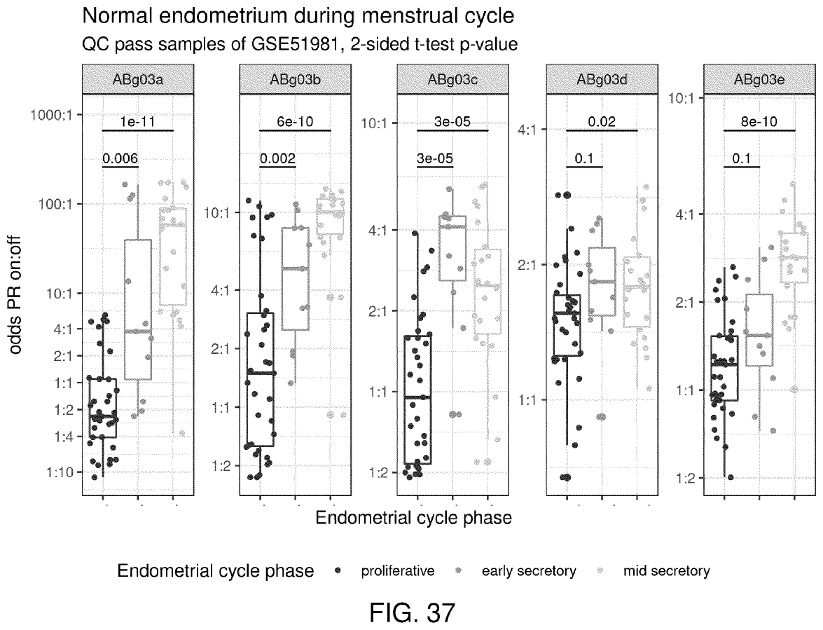

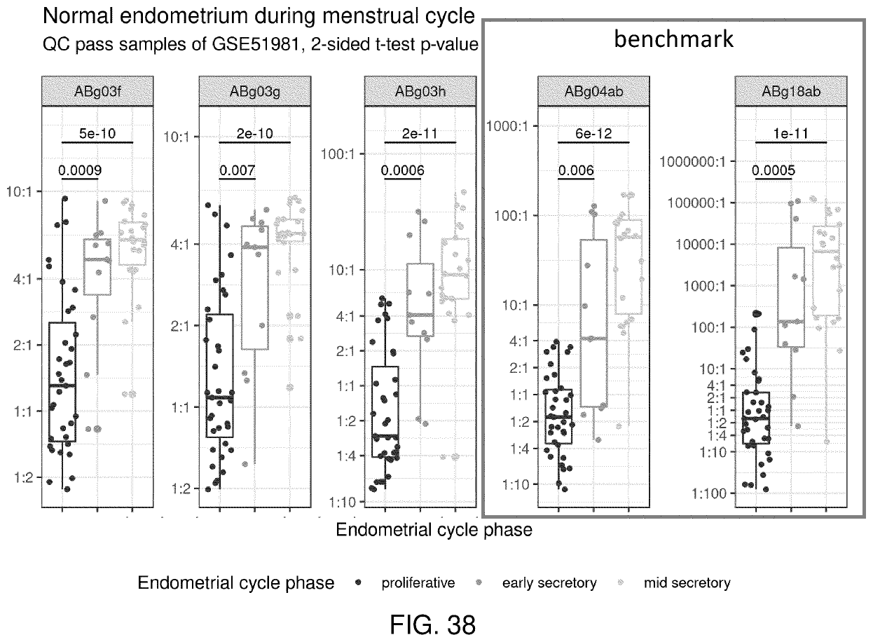

FIG. 37 shows PR-AB cellular pathway activity predictions of trained Bayesian network models calibrated with alternative target gene lists comprising 3 randomly selected genes from the preferred PR-AB target gene set (see Table 8), and the preferred calibration set EmLPxHS (see Table 4). The activities were inferred for normal endometrium during menstrual cycle QC pass samples from GSE51981 (see FIG. 10). From left to right the following gene sets are used: ABg03a (FKBP5, BCL6, S100P), ABg03b (BCL6, NFKBIA, ACSS1), ABg03c (PDK4, NFKBIA, KANK1), ABg03d (ABCG2, GRB10, ACSS1), and ABg03e (CCND1, TSC223D, ATP1B1). The resulting PR-AB cellular signaling pathway activities are substantially comparable to the results obtained with the preferred target gene lists (benchmark, see FIG. 38).

FIG. 38 shows PR-AB cellular pathway activity predictions of trained Bayesian network models calibrated with alternative target gene lists comprising 3 randomly selected genes from the preferred PR-AB target gene set (see Table 8), and the preferred calibration set EmLPxHS (see Table 4). The activities were inferred for normal endometrium during menstrual cycle QC pass samples from GSE51981 (see FIG. 10). From left to right the following gene sets are used: ABg03f (GRB10, PLIN2, VASP), ABg03g (PLIN2, VASP, ACSS1), ABg03h (BCL6, ATP1B1, AK4), ABg04ab (all genes listed in Table 13) and ABg18ab (all genes listed in Table 8). The resulting PR-AB cellular signaling pathway activities are substantially comparable to the results obtained with the preferred target gene lists (benchmark).

The experimental results can be summarized as follows:

(1) PR cellular signaling pathway models correctly infer increase in PR cellular signaling pathway activity in progesterone sensitive tissue and cell lines exposed to progestogens.

(a) Normal reproductive tissue samples during menstrual cycle: The concentration of progesterone (P4) in the endometrium varies considerably during the menstrual cycle from near absent levels in the proliferative phase of the endometrium cycle to high levels of the secretory phase of the endometrium cycle (with mean peak values in the order of 13 ng/mL). FIG. 8 shows that all preferred PR-A, PR-B and PR-A&B target gene lists correctly infer an increase in PR cellular signaling pathway activity levels with an increase of the progesterone level and that PR cellular signaling pathway activity is highly correlated with progesterone levels. FIGS. 9 and 10 show that all preferred PR-A, PR-B and PR-A&B target gene lists correctly infer an increase in PR cellular signaling pathway activity levels as the endometrium goes from the proliferative phase to the secretory phase. The follicular and luteal phases of the ovarian cycle corresponds to the proliferative phase and secretory phases of the endometrium cycle, where progesterone levels are, respectively, the lowest and highest (see FIG. 11). FIG. 12 shows that all preferred PR-A, PR-B and PR-A&B target gene lists correctly infer an increase in PR cellular signaling pathway activity levels in fallopian tube tissue as the cycle moves from the follicular to the luteal phase. However, in this case, only the PR-B and PR-A&B models achieve statistical significance.

(b) Other progesterone sensitive tissue and cell lines exposed to progestogens: FIGS. 13 to 16 illustrate other exemplary cases where the preferred PR-A, PR-B and PR-A&B target gene lists correctly infer an increase in PR cellular signaling pathway activity when progesterone sensitive tissues or cell lines are stimulated with either the synthetic progestin MPA or the synthetic progestin R5020.

(2) In cancer and other morbidities, similarly to PR-A and PR-B protein levels, PR-A and PR-B cellular signaling pathway inferred activity levels are frequently disrupted.

(a) PR is disrupted in breast cancer: Mote, P. A. et al., “Progesterone Receptor A Predominance Is a Discriminator of Benefit from Endocrine Therapy in the ATAC Trial”, Breast Cancer Research and Treatment, Vol. 151, No. 2, 2015, pages 309-318 have shown that in breast cancer PR-A and PR-B protein levels are disrupted. PR-A is generally predominant but PR-B may also be predominant. Accordingly, FIG. 17 shows that, compared to normal tissue, PR-A and PR-B cellular signaling pathway activity levels are disrupted in breast cancer tissue. While normalized PR-A and PR-B cellular signaling pathway activity levels are similar or PR-B is higher than PR-A in normal issue (see FIG. 17 “a”), normalized PR-B cellular signaling pathway activity levels are generally lower than normalized PR-A cellular signaling pathway activity levels in tumor tissue (see FIG. 17 “b”). In most cases, normalized PR-A cellular signaling pathway activity levels are higher in tumor than in normal samples (see FIG. 17 “c”) and normalized PR-B cellular signaling pathway activity levels are lower in tumor than in normal samples (see FIG. 17 “d”).

(b) PR is disrupted in endometrial cancer: In endometrial cancer, Arnett-Mansfield, R. L. et al., “Subnuclear Distribution of Progesterone Receptors A and B in Normal and Malignant Endometrium”, The Journal of Clinical Endocrinology & Metabolism Vol. 89, No. 3, 2004, pages 1429-1442 also showed a disruption between PR-A and PR-B protein isoform expression in cancer compared to the normal endometrium. FIG. 18 shows that PR-A cellular signaling pathway activity levels are increased in endometrial cancer samples compared to normal samples and PR-B cellular signaling pathway activity levels are similar or decreased in endometrial cancer samples compared to normal samples, wherein the difference between PR-A and PR-B increases with cancer grade.

(c) PR is also disrupted in other cancer types as exemplified in FIGS. 19 and 20: In lung cancer, while PR-A cellular signaling pathway activity levels increase significantly in cancer compared to normal tissue, PR-B and PR-A&B levels are significantly reduced. Disruption in PR-A and PR-B cellular signaling pathway activity levels are larger on more severe stages of lung cancer.

(d) PR is also disrupted in other morbidities as exemplified in FIGS. 21 and 27.

(3) PR activity as a monitor of therapy response: FIG. 22 shows that in ER positive breast cancer patients treated with neoadjuvant aromatase inhibitor letrozole, mean PR-B cellular signaling pathway activity levels are higher in responders than in non-responders. Mean PR-B cellular signaling pathway activity levels go up in responders with activity levels approaching PR-B cellular signaling pathway activity in normal samples (see FIG. 16). Activity levels remain similar to pre-treatment in non-responders.

(4) PR activity as prognostic risk marker: The survival curves of FIG. 23 exemplify PR-A, PR-B and ratios computed from normalized PR-A, PR-B and PR-A&B cellular signaling pathway activity levels as significant prognostic factors for the progression of lung cancer. The survival curves of FIG. 24 exemplify combinations of PR cellular signaling pathway activity levels and PR cellular signaling pathway activity level ratios that are significant prognostic factors for the progression of lung cancer. FIG. 25 exemplifies combinations of PR cellular signaling pathway activity levels and PR cellular signaling pathway activity level ratios that are significant prognostic factors for the progression of breast cancer. FIG. 26 exemplifies PR cellular signaling pathway activity levels as a prognostic risk factor for the progression of pediatric acute lymphoblastic leukemia (ALL).

(5) PR cellular signaling pathway activity levels as a risk factor: FIG. 27 exemplifies PR cellular signaling pathway activity levels as a prognostic risk factor in breast cancer.

(6) PR cellular signaling pathway activity levels as response predictor: The survival curves in FIG. 28 and the cox proportional hazard regression summary exemplify the use of PR cellular signaling pathway models as a predictor to progestogen therapy response in endometrial cancer and its added value over use of ER immunohistochemistry (IHC) only.

(7) Variations of the PR cellular signaling pathway model: PR cellular signaling pathway models can also be constructed using alternative calibration sets as exemplified in FIGS. 29 and 30 and using alternative target gene lists as exemplified in FIGS. 31 and 32. FIG. 28 exemplifies results obtained by inferring activities by simply adding the normalized expressions of target genes (i.e., using a linear combination) obtained using RT-qPCR.

(8) Using random selections of sets of three genes from preferred sets of genes for PR-A, PR-B and PR-AB it is demonstrated that PR cellular signaling pathway models can also be constructed using a minimum of three genes from the lists of genes, as exemplified in FIGS. 33 to 38. All randomly selected sets of three genes provided similar results as the benchmark (full set of preferred genes).

Instead of applying the calibrated mathematical model, e.g., the exemplary Bayesian network model, on mRNA input data coming from microarrays or RNA sequencing, it may be beneficial in clinical applications to develop dedicated assays to perform the sample measurements, for instance on an integrated platform using qPCR to determine mRNA levels of target genes. The RNA/DNA sequences of the disclosed target genes can then be used to determine which primers and probes to select on such a platform.

Validation of such a dedicated assay can be done by using the microarray-based mathematical model as a reference model, and verifying whether the developed assay gives similar results on a set of validation samples. Next to a dedicated assay, this can also be done to build and calibrate similar mathematical models using RNA sequencing data as input measurements.

The set of target genes which are found to best indicate specific cellular signaling pathway activity based on microarray/RNA sequencing based investigation using the calibrated mathematical model, e.g., the exemplary Bayesian network model, can be translated into a multiplex quantitative PCR assay to be performed on a sample of the subject and/or a computer to interpret the expression measurements and/or to infer the activity of the PR cellular signaling pathway. To develop such a test (e.g., FDA-approved or a CLIA waived test in a central service lab or a laboratory developed test for research use only) for cellular signaling pathway activity, development of a standardized test kit is required, which needs to be clinically validated in clinical trials to obtain regulatory approval.

The present invention relates to a computer-implemented method for inferring activity of a PR cellular signaling pathway in a subject performed by a digital processing device, wherein the inferring is based on expression levels of three or more target genes of the PR cellular signaling pathway measured in a sample of the subject. The present invention further relates to an apparatus for inferring activity of a PR cellular signaling pathway in a subject comprising a digital processor configured to perform the method, to a non-transitory storage medium for inferring activity of a PR cellular signaling pathway in a subject storing instructions that are executable by a digital processing device to perform the method, and to a computer program for inferring activity of a PR cellular signaling pathway in a subject comprising program code means for causing a digital processing device to perform the method, when the computer program is run on the digital processing device.

The method may be used, for instance, in diagnosing an (abnormal) activity of the PR cellular signaling pathway, in prognosis based on the inferred activity of the PR cellular signaling pathway, in the enrollment of a subject in a clinical trial based on the inferred activity of the PR cellular signaling pathway, in the selection of subsequent test(s) to be performed, in the selection of companion diagnostics tests, in clinical decision support systems, or the like. In this regard, reference is made to the published international patent application WO 2013/011479 A2 (“Assessment of cellular signaling pathway activity using probabilistic modeling of target gene expression”), to the published international patent application WO 2014/102668 A2 (“Assessment of cellular signaling pathway activity using linear combination(s) of target gene expressions”), and to Verhaegh W. et al., “Selection of personalized patient therapy through the use of knowledge-based computational models that identify tumor-driving signal transduction pathways”, Cancer Research, Vol. 74, No. 11, 2014, pages 2936 to 2945, which describe these applications in more detail.

### 5. Further Information for Illustrating the Present Invention

### (1) Measuring Levels of Gene Expression

Data derived from the unique set of target genes described herein is further utilized to infer an activity of the PR cellular signaling pathway using the methods described herein.

Methods for analyzing gene expression levels in extracted samples are generally known. For example, methods such as Northern blotting, the use of PCR, nested PCR, quantitative real-time PCR (qPCR), RNA-seq, or microarrays can all be used to derive gene expression level data. All methods known in the art for analyzing gene expression of the target genes are contemplated herein.

Methods of determining the expression product of a gene using PCR based methods may be of particular use. In order to quantify the level of gene expression using PCR, the amount of each PCR product of interest is typically estimated using conventional quantitative real-time PCR (qPCR) to measure the accumulation of PCR products in real time after each cycle of amplification. This typically utilizes a detectible reporter such as an intercalating dye, minor groove binding dye, or fluorogenic probe whereby the application of light excites the reporter to fluoresce and the resulting fluorescence is typically detected using a CCD camera or photomultiplier detection system, such as that disclosed in U.S. Pat. No. 6,713,297 which is hereby incorporated by reference.

In some embodiments, the probes used in the detection of PCR products in the quantitative real-time PCR (qPCR) assay can include a fluorescent marker. Numerous fluorescent markers are commercially available. For example, Molecular Probes, Inc. (Eugene, Oreg.) sells a wide variety of fluorescent dyes. Non-limiting examples include Cy5, Cy3, TAN/IRA, R6G, R110, ROX, JOE, FAM, Texas Red™, and Oregon Green™ Additional fluorescent markers can include IDT ZEN Double-Quenched Probes with traditional 5′ hydrolysis probes in qPCR assays. These probes can contain, for example, a 5′ FAM dye with either a 3′ TAMRA Quencher, a 3′ Black Hole Quencher (BHQ, Biosearch Technologies), or an internal ZEN Quencher and 3′ Iowa Black Fluorescent Quencher (IBFQ).

Fluorescent dyes useful according to the invention can be attached to oligonucleotide primers using methods well known in the art. For example, one common way to add a fluorescent label to an oligonucleotide is to react an N-Hydroxysuccinimide (NETS) ester of the dye with a reactive amino group on the target. Nucleotides can be modified to carry a reactive amino group by, for example, inclusion of an allyl amine group on the nucleobase. Labeling via allyl amine is described, for example, in U.S. Pat. Nos. 5,476,928 and 5,958,691, which are incorporated herein by reference. Other means of fluorescently labeling nucleotides, oligonucleotides and polynucleotides are well known to those of skill in the art.

Other fluorogenic approaches include the use of generic detection systems such as SYBR-green dye, which fluoresces when intercalated with the amplified DNA from any gene expression product as disclosed in U.S. Pat. Nos. 5,436,134 and 5,658,751 which are hereby incorporated by reference.

Another useful method for determining target gene expression levels includes RNA-seq, a powerful analytical tool used for transcriptome analyses, including gene expression level difference between different physiological conditions, or changes that occur during development or over the course of disease progression.

Another approach to determine gene expression levels includes the use of microarrays for example RNA and DNA microarray, which are well known in the art. Microarrays can be used to quantify the expression of a large number of genes simultaneously.

### (2) Generalized Workflow for Determining the Activity of PR Cellular Signaling

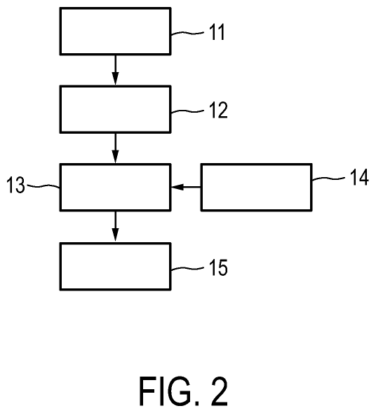

A flowchart exemplarily illustrating a process for inferring the activity of PR cellular signaling from a sample isolated from a subject is shown in FIG. 2. First, the mRNA from a sample is isolated (11). Second, the mRNA expression levels of a unique set of at least three or more PR target genes, as described herein, are measured (12) using methods for measuring gene expression that are known in the art. Next, an activity level of a PR transcription factor (TF) element (13) is determined using a calibrated mathematical pathway model (14) relating the expression levels of the three or more PR target genes to the activity level of the PR TF element. Finally, the activity of the PR cellular signaling pathway in the subject is inferred (15) based on the determined activity level of the PR TF element in the sample of the subject. For example, the PR cellular signaling pathway is determined to be active if the activity is above a certain threshold, and can be categorized as passive if the activity falls below a certain threshold.

### (3) Calibrated Mathematical Pathway Model

As contemplated herein, the expression levels of the unique set of three or more PR target genes described herein are used to determine an activity level of a PR TF element using a calibrated mathematical pathway model as further described herein. The calibrated mathematical pathway model relates the expression levels of the three or more PR target genes to the activity level of the PR TF element.

As contemplated herein, the calibrated mathematical pathway model is based on the application of a mathematical pathway model. For example, the calibrated mathematical pathway model can be based on a probabilistic model, for example, a Bayesian network model, or a linear or pseudo-linear model.

In an embodiment, the calibrated mathematical pathway model is a probabilistic model incorporating conditional probabilistic relationships relating the PR TF element and the expression levels of the three or more PR target genes. In an embodiment, the probabilistic model is a Bayesian network model.

In an alternative embodiment, the calibrated pathway mathematical model can be a linear or pseudo-linear model. In an embodiment, the linear or pseudo-linear model is a linear or pseudo-linear combination model as further described herein.

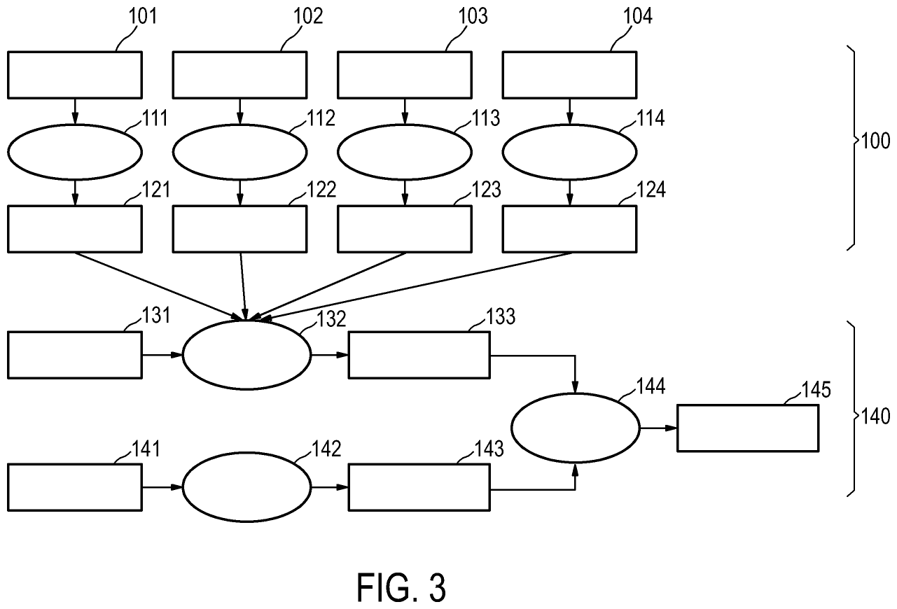

A flowchart exemplarily illustrating a process for generating a calibrated mathematical pathway model is shown in FIG. 3. As an initial step, the training data for the mRNA expression levels is collected and normalized. The data can be collected using, for example, microarray probeset intensities (101), real-time PCR Cq values (102), raw RNAseq reads (103), or alternative measurement modalities (104) known in the art. The raw expression level data can then be normalized for each method, respectively, by normalization using a normalization algorithm, for example, frozen robust multiarray analysis (fRMA) or MAS5.0 (111), normalization to average Cq of reference genes (112), normalization of reads into reads/fragments per kilobase of transcript per million mapped reads (RPKM/FPKM) (113), or normalization w.r.t. reference genes/proteins (114). This normalization procedure leads to a normalized probeset intensity (121), normalized Cq values (122), normalized RPKM/FPKM (123), or normalized measurement (124) for each method, respectively, which indicate target gene expression levels within the training samples.

Once the training data has been normalized, a training sample ID or IDs (131) is obtained and the training data of these specific samples is obtained from one of the methods for determining gene expression (132). The final gene expression results from the training sample are output as training data (133). All of the data from various training samples are incorporated to calibrate the model (including for example, thresholds, CPTs, for example in the case of the probabilistic or Bayesian network, weights, for example, in the case of the linear or pseudo-linear model, etc) (144). In addition, the pathway's target genes and measurement nodes (141) are used to generate the model structure for example, as described in FIG. 1 (142). The resulting model structure (143) of the pathway is then incorporated with the training data (133) to calibrate the model (144), wherein the gene expression levels of the target genes is indicative of the transcription factor element activity. As a result of the TF element determination in the training samples, a calibrated pathway model (145) is generated, which assigns the PR cellular signaling pathway activity for a subsequently examined sample of interest, for example from a subject with a cancer, based on the target gene expression levels in the training samples.

### (4) TF Element Determination

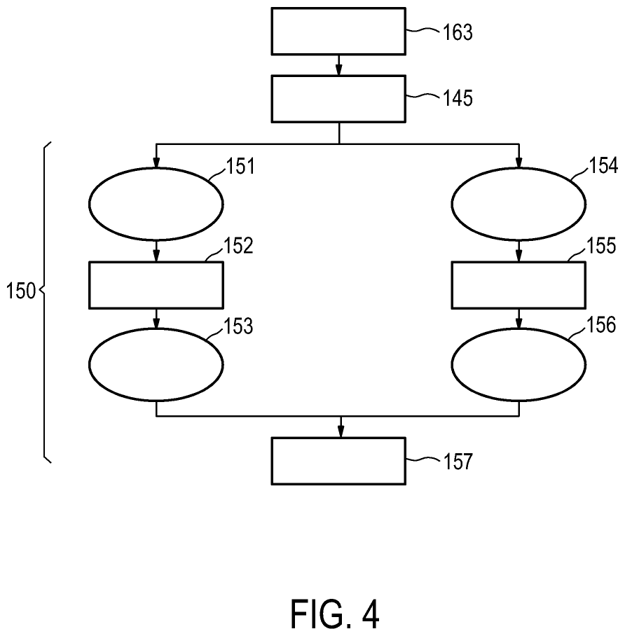

A flowchart exemplarily illustrating a process for determining an activity level of a TF element is shown in FIG. 4. The expression level data (test data) (163) from a sample extracted from a subject is input into the calibrated mathematical pathway model (145). The mathematical pathway model may be a probabilistic model, for example, a Bayesian network model, a linear model, or a pseudo-linear model.

The mathematical pathway model may be a probabilistic model, for example, a Bayesian network model, based on conditional probabilities relating the PR TF element and expression levels of the three or more target genes of the PR cellular signaling pathway measured in the sample of the subject, or the mathematical model may be based on one or more linear combination(s) of expression levels of the three or more target genes of the PR cellular signaling pathway measured in the sample of the subject. In particular, the determining of the activity of the PR cellular signaling pathway may be performed as disclosed in the published international patent application WO 2013/011479 A2 (“Assessment of cellular signaling pathway activity using probabilistic modeling of target gene expression”), the contents of which are herewith incorporated in their entirety. Briefly, the data is entered into a Bayesian network (BN) inference engine call (for example, a BNT toolbox) (154). This leads to a set of values for the calculated marginal BN probabilities of all the nodes in the BN (155). From these probabilities, the transcription factor (TF) node's probability (156) is determined and establishes the TF element's activity level (157).

Alternatively, the mathematical model may be a linear model. For example, a linear model can be used as described in the published international patent application WO 2014/102668 A2 (“Assessment of cellular signaling pathway activity using linear combination(s) of target gene expressions”), the contents of which are herewith incorporated in their entirety. Further details regarding the calculating/determining of cellular signaling pathway activity using mathematical modeling of target gene expression can also be found in Verhaegh W. et al., “Selection of personalized patient therapy through the use of knowledge-based computational models that identify tumor-driving signal transduction pathways”, Cancer Research, Vol. 74, No. 11, 2014, pages 2936-2945. Briefly, the data is entered into a calculated weighted linear combination score (w/c) (151). This leads to a set of values for the calculated weighted linear combination score (152). From these weighted linear combination scores, the transcription factor (TF) node's weighted linear combination score (153) is determined and establishes the TF's element activity level (157).

### (5) Procedure for Discretized Observables

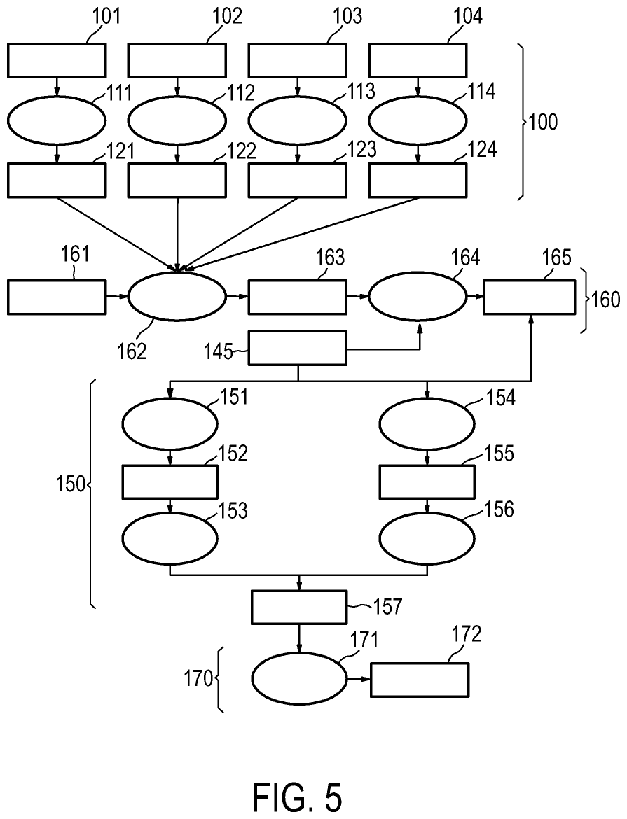

A flowchart exemplarily illustrating a process for inferring activity of a PR cellular signaling pathway in a subject as a discretized observable is shown in FIG. 5. First, the test sample is extracted and given a test sample ID (161). Next, the test data for the mRNA expression levels is collected and normalized (162). The test data can be collected using the same methods as discussed for the training samples in FIG. 4, using microarray probeset intensities (101), real-time PCR Cq values (102), raw RNAseq reads (103), or an alternative measurement modalities (104). The raw expression level data can then be normalized for each method, respectively, by normalization using an algorithm, for example fRMA or MAS5.0 (111), normalization to average Cq of reference genes (112), normalization of reads into RPKM/FPKM (113), and normalization w.r.t. reference genes/proteins (114). This normalization procedure leads to a normalized probeset intensity (121), normalized Cq values (122), normalized RPKM/FPKM (123), or normalized measurement (124) for each method, respectively.

Once the test data has been normalized, the resulting test data (163) is analyzed in a thresholding step (164) based on the calibrated mathematical pathway model (145), resulting in the thresholded test data (165). In using discrete observables, in one non-limiting example, every expression above a certain threshold is, for example, given a value of 1 and values below the threshold are given a value of 0, or in an alternative embodiment, the probability mass above the threshold as described herein is used as a thresholded value. Based on the calibrated mathematical pathway model, this value represents the TF element's activity level (157), which is then used to calculate the cellular signaling pathway's activity (171). The final output gives the cellular signaling pathway's activity (172) in the subject.

### (6) Procedure for Continuous Observables

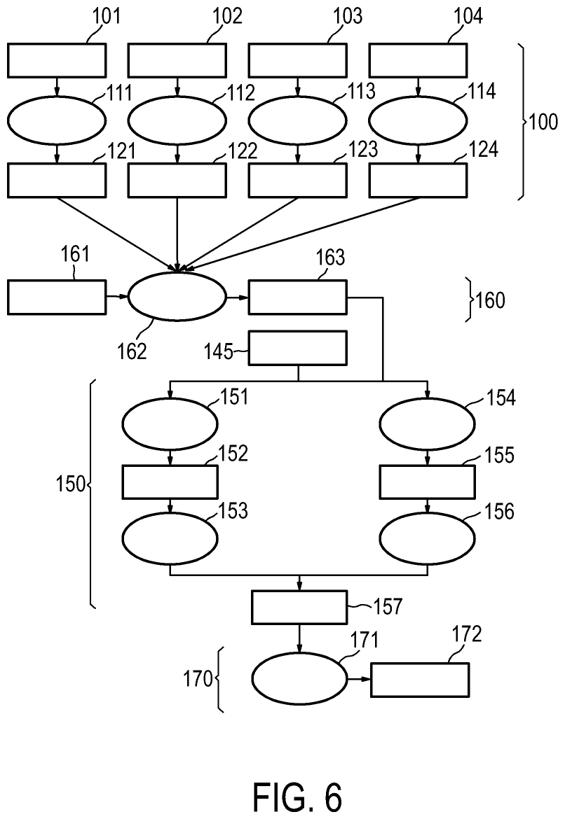

A flowchart exemplarily illustrating a process for inferring activity of a PR cellular signaling pathway in a subject as a continuous observable is shown in FIG. 6. First, the test sample is extracted and given a test sample ID (161). Next, the test data for the mRNA expression levels is collected and normalized (162). The test data can be collected using the same methods as discussed for the training samples in FIG. 5, using microarray probeset intensities (101), real-time PCR Cq values (102), raw RNAseq reads (103), or an alternative measurement modalities (104). The raw expression level data can then be normalized for each method, respectively, by normalization using an algorithm, for example fRMA (111), normalization to average Cq of reference genes (112), normalization of reads into RPKM/FPKM (113), and normalization w.r.t. reference genes/proteins (114). This normalization procedure leads to a normalized probeset intensity (121), normalized Cq values (122), normalized RPKM/FPKM (123), or normalized measurement (124) for each method, respectively.

Once the test data has been normalized, the resulting test data (163) is analyzed in the calibrated mathematical pathway model (145). In using continuous observables, as one non-limiting example, the expression levels are converted to values between 0 and 1 using a sigmoid function as described in further detail herein. The TF element determination as described herein is used to interpret the test data in combination with the calibrated mathematical pathway model, the resulting value represents the TF element's activity level (157), which is then used to calculate the cellular signaling pathway's activity (171). The final output gives the cellular signaling pathway's activity (172) in the subject.

### (7) Target Gene Expression Level Determination Procedure

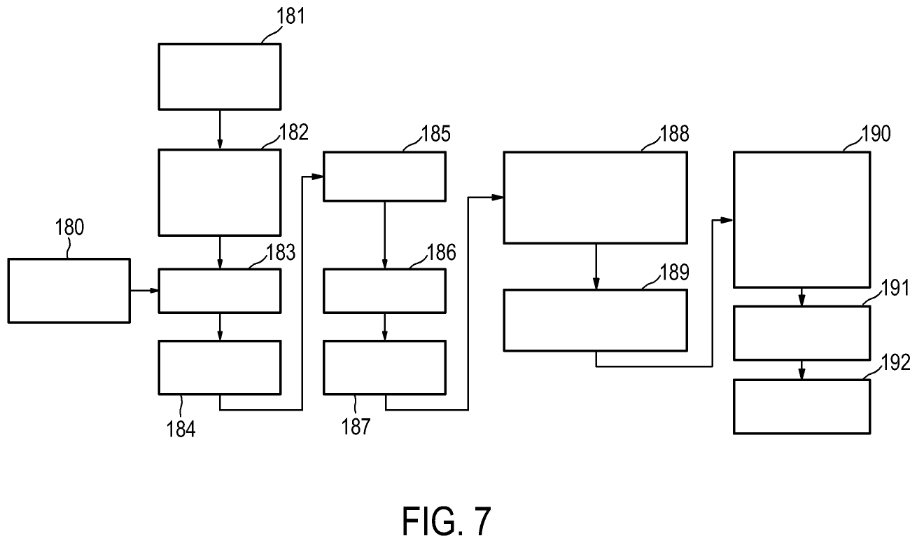

A flowchart exemplary illustrating a process for deriving target gene expression levels from a sample extracted from a subject is shown in FIG. 7. In an exemplary embodiment, samples are received and registered in a laboratory. Samples can include, for example, Formalin-Fixed, Paraffin-Embedded (FFPE) samples (181) or fresh frozen (FF) samples (180). FF samples can be directly lysed (183). For FFPE samples, the paraffin can be removed with a heated incubation step upon addition of Proteinase K (182). Cells are then lysed (183), which destroys the cell and nuclear membranes which makes the nucleic acid (NA) available for further processing. The nucleic acid is bound to a solid phase (184) which could for example, be beads or a filter. The nucleic acid is then washed with washing buffers to remove all the cell debris which is present after lysis (185). The clean nucleic acid is then detached from the solid phase with an elution buffer (186). The DNA is removed by DNAse treatment to ensure that only RNA is present in the sample (187). The nucleic acid sample can then be directly used in the RT-qPCR sample mix (188). The RT-qPCR sample mixes contains the RNA sample, the RT enzyme to prepare cDNA from the RNA sample and a PCR enzyme to amplify the cDNA, a buffer solution to ensure functioning of the enzymes and can potentially contain molecular grade water to set a fixed volume of concentration. The sample mix can then be added to a multiwell plate (i.e., 96 well or 384 well plate) which contains dried RT-qPCR assays (189). The RT-qPCR can then be run in a PCR machine according to a specified protocol (190). An example PCR protocol includes i) 30 minutes at 50° C.; ii) 5 minutes at 95° C.; iii) 15 seconds at 95° C.; iv) 45 seconds at 60° C.; v) 50 cycles repeating steps iii and iv. The Cq values are then determined with the raw data by using the second derivative method (191). The Cq values are exported for analysis (192).

### (8) PR Mediated Diseases and Disorders and Methods of Treatment

As contemplated herein, the methods and apparatuses of the present invention can be utilized to assess PR cellular signaling pathway activity in a subject, for example, a subject suspected of having, or having, a disease or disorder wherein the status of the PR signaling pathway is probative, either wholly or partially, of disease presence or progression. In an embodiment, provided herein is a method of treating a subject comprising receiving information regarding the activity status of a PR cellular signaling pathway derived from a sample extracted from the subject using the methods described herein and administering to the subject a PR inhibitor if the information regarding the activity of the PR cellular signaling pathway is indicative of an active PR signaling pathway. In a particular embodiment, the PR cellular signaling pathway activity indication is set at a cutoff value of odds of the PR cellular signaling pathway being active of 10:1, 5:1, 4:1, 2:1, 1:1, 1:2, 1:4, 1:5, 1:10.

PR inhibitors that may be used in the present invention are well known. Examples of PR inhibitors include, but are not limited to, mifepristone (MFP; RU-486), Bisphenol A: (BPA), Asoprisnil. Likewise, PR agonists that may be used in the present invention are well-known. Examples of PR agonists include, but are not limited to, Progesterone (P4), Org2058, promegestone (R5020), medroxyprogesterone acetate (MPA).

In a particular embodiment, the subject is suffering, or suspected to be suffering from, a breast cancer, an endometrial cancer, an ovarian cancer, a lung cancer or an acute lymphoblastic leukemia (ALL) cancer. In a particular embodiment, the subject is suffering from, or suspected to be suffering from, a breast cancer.

This application describes several preferred embodiments. Modifications and alterations may occur to others upon reading and understanding the preceding detailed description. It is intended that the application is construed as including all such modifications and alterations insofar as they come within the scope of the appended claims or the equivalents thereof.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Calculations like the determination of the risk score performed by one or several units or devices can be performed by any other number of units or devices.

A computer program may be stored/distributed on a suitable medium, such as an optical storage medium or a solid-state medium, supplied together with or as part of other hardware, but may also be distributed in other forms, such as via the Internet or other wired or wireless telecommunication systems.

### 6. Sequence Listings Used in Application

SEQUENCE LISTING:

| Seq. No. | Gene: |
|---|---|
| Seq. 1 | ABCG2 |
| Seq. 2 | ACSS1 |
| Seq. 3 | AK4 |
| Seq. 4 | ARRDC1 |
| Seq. 5 | ATP1B1 |
| Seq. 6 | BCL2L1 |
| Seq. 7 | BCL6 |
| Seq. 8 | BIRC3 |
| Seq. 9 | CCND1 |
| Seq. 10 | CD82 |
| Seq. 11 | CDKN1A |
| Seq.12 | DDIT4 |
| Seq.13 | E2F1 |
| Seq.14 | F3 |
| Seq. 15 | FKBP5 |
| Seq. 16 | GOT1 |
| Seq. 17 | GRB10 |
| Seq. 18 | HPCAL1 |
| Seq. 19 | HSD11B2 |
| Seq. 20 | KANK1 |
| Seq. 21 | KLF4 |
| Seq. 22 | MSX2 |
| Seq. 23 | MUC1 |
| Seq. 24 | MYC |
| Seq. 25 | NEDD9 |
| Seq. 26 | NET1 |
| Seq. 27 | NFKBIA |
| Seq. 28 | PDK4 |
| Seq. 29 | PLIN2 |
| Seq. 30 | PTP4A2 |
| Seq. 31 | S100P |
| Seq. 32 | SGK1 |
| Seq. 33 | SNTB2 |
| Seq. 34 | STAT5A |
| Seq. 35 | TRIM22 |
| Seq. 36 | TSC22D3 |
| Seq. 37 | VASP |
| Seq. 38 | VEGFA |

1.-15. (canceled)

16. A method of treatment, the method comprising:
inferring activity of a PR cellular signaling pathway in a subject, wherein the inferring comprises:
receiving expression levels of three or more target genes of the PR cellular signaling pathway measured in a sample of the subject,
determining an activity level of a PR transcription factor (TF) element in the sample of the subject, the PR TF element controlling transcription of the three or more PR target genes, the determining being based on evaluating a calibrated mathematical pathway model relating the expression levels of the three or more PR target genes to the activity level of the PR TF element, and
inferring the activity of the PR cellular signaling pathway in the subject based on the determined activity level of the PR TF element in the sample of the subject, and
determining that the PR cellular signaling pathway is operating abnormally in the subject based on the inferred activity of the PR cellular signaling pathway in the subject, and
treating said subject by administering an agonist of PR pathway activity when the PR cellular signaling pathway is determined to be operating with abnormally low activity, or treating said patient by administering an inhibitor of PR pathway activity when the PR cellular signaling pathway is determined to be operating with abnormally high activity.

17. The method of claim 16, wherein the calibrated mathematical pathway model is PR-A specific and the three or more target genes are selected from the group consisting of: BCL2L1, BIRC3, DDIT4, F3, MUC1, NEDD9, SGK1, and TRIM22, preferably, from the group consisting of: BCL2L1, DDIT4, NEDD9, and TRIM22.

18. The method according to claim 16, wherein the calibrated mathematical pathway model is PR-B specific and the three or more target genes are selected from the group consisting of: ARRDC1, ATP1B1, BIRC3, CCND1, CD82, DDIT4, E2F1, F3, FKBP5, GOT1, HSD11B2, KANK1, MSX2, MUC1, MYC, NET1, NFKBIA, PDK4, PLIN2, PTP4A2, SNTB2, and STAT5A, preferably, from the group consisting of: ARRDC1, ATP1B1, CCND1, CD82, E2F1, FKBP5, GOT1, HSD11B2, KANK1, MSX2, MYC, NET1, NFKBIA, PDK4, PLIN2, PTP4A2, SNTB2, and STAT5A, preferably, from the group consisting of: ARRDC1, ATP1B1, CCND1, E2F1, FKBP5, HSD11B2, KANK1, MSX2, MYC, NET1, NFKBIA, PDK4, and PLIN2, preferably, from the group consisting of: CCND1, FKBP5, and MYC.

19. The method according to claim 16, wherein the calibrated mathematical pathway model is PR-A&B specific and the three or more target genes are selected from the group consisting of: ABCG2, ACSS1, AK4, ARRDC1, ATP1B1, BCL2L1, BCL6, BIRC3, CCND1, CD82, CDKN1A, DDIT4, E2F1, F3, FKBP5, GOT1, GRB10, HPCAL1, HSD11B2, KANK1, KLF4, MSX2, MUC1, MYC, NEDD9, NET1, NFKBIA, PDK4, PLIN2, PTP4A2, S100P, SGK1, SNTB2, STAT5A, TRIM22, TSC22D3, VASP, and VEGFA, preferably, from the group consisting of: AK4, ARRDC1, ATP1B1, BCL2L1, BCL6, BIRC3, CCND1, CD82, F3, FKBP5, GOT1, GRB10, HSD11B2, KLF4, MUC1, MYC, NEDD9, NET1, PDK4, PTP4A2, S100P, SGK1, SNTB2, STAT5A, TSC22D3, and VASP, or, preferably, from the group consisting of: ABCG2, ACSS1, AK4, ATP1B1, BCL6, CCND1, FKBP5, GRB10, HSD11B2, KANK1, KLF4, MYC, NFKBIA, PDK4, PLIN2, S100P, TSC22D3, and VASP, or, preferably, from the group consisting of: BCL6, CCND1, CDKN1A, FKBP5, MYC, SGK1, and VEGFA, or, preferably, from the group consisting of: BCL6, CCND1, FKBP5, and MYC.

20. The method according to claim 16, wherein the subject is suffering from cancer.

21. The method according to claim 20, wherein the cancer is breast cancer, endometrial cancer, ovarian cancer, lung cancer, or acute lymphoblastic leukemia (ALL).

22. The method of claim 16, wherein the subject is suspected of having a disease or disorder, and wherein the status of the PR signaling pathway is probative of disease presence or progression.

23. The method of claim 16, wherein the subject has a disease or disorder, and wherein the status of the PR signaling pathway is probative of disease presence or progression.

24. The method of claim 16, wherein said treatment comprises administering a PR inhibitor to the subject.

25. The method of claim 24 wherein the PR inhibitor is selected from mifepristone (MFP; RU-486), Bisphenol A: (BPA), and Asoprisnil.

26. The method of claim 16 wherein the treatment comprises administerting a PR agonist to the subject.

27. The method of claim 26, wherein the PR agonist is selected from Progesterone (P4), Org2058, promegestone (R5020), and medroxyprogesterone acetate (MPA).

28. The method of claim 16, further comprising:
determining a prognostic cancer marker based on a combination of inferred activities of the PR cellular signaling pathway in the subject using two or more of the PR-A specific calibrated mathematical pathway model, the PR-B specific calibrated mathematical pathway model, and the PR-A&B specific calibrated mathematical pathway model.

29. The method of claim 28, wherein the combination is a ratio between the inferred activity of the PR cellular signaling pathway in the subject using the PR-A&B specific calibrated mathematical pathway model and the inferred activity of the PR cellular signaling pathway in the subject using the PR-B specific calibrated mathematical pathway model.

30. The method of claim 29, further comprising:
recommending prescribing a drug for the subject that corrects for abnormal operation of the PR cellular signaling pathway,
wherein the recommending is performed when the PR cellular signaling pathway is determined to be operating abnormally in the subject based on the inferred activity of the PR cellular signaling pathway.

31. The method of claim 29, wherein the abnormal operation of the PR cellular signaling pathway is an operation in which the PR cellular signaling pathway operates as a tumor promoter in the subject.

32. The method of claim 16, wherein the method is used in at least one of the following activities:
diagnosis based on the inferred activity of the PR cellular signaling pathway in the subject;
prognosis based on the inferred activity of the PR cellular signaling pathway in the subject;
drug prescription based on the inferred activity of the PR cellular signaling pathway in the subject;
prediction of drug efficacy based on the inferred activity of the PR cellular signaling pathway in the subject;
prediction of adverse effects based on the inferred activity of the PR cellular signaling pathway in the subject;
monitoring of drug efficacy;
drug development;
assay development;
pathway research;
cancer staging;
enrollment of the subject in a clinical trial based on the inferred activity of the PR cellular signaling pathway in the subject;
selection of subsequent test to be performed; and
selection of companion diagnostics tests.

33. The method of claim 16, wherein the calibrated mathematical pathway model is a probabilistic model, preferably a Bayesian network model, based on conditional probabilities relating the activity level of the PR TF element and the expression levels of the three or more PR target genes, or wherein the mathematical pathway model is based on one or more linear combination(s) of the expression levels of the three or more PR target genes.
